SEQUENCE LISTING (6 sequences)

SEQ ID NO: 1 - protein, 9 aa, artificial sequence
  HLA-A*02 TCR.90
NLVPMVATV

SEQ ID NO: 2 - protein, 10 aa, artificial sequence
  HLA-B*07 TCR.28
TPRVTGGGAM

SEQ ID NO: 3 - protein, 9 aa, artificial sequence
  HLA-B*35 TCR.6
IPSINVHHY

SEQ ID NO: 4 - protein, 11 aa, artificial sequence
  HLA-B*07 TCR.6
RPHERNGFTVL

SEQ ID NO: 5 - protein, 12 aa, artificial sequence
  HLA-B*35 TCR.1
CPSQEPMSIYVY

SEQ ID NO: 6 - protein, 11 aa, artificial sequence
  HLA-B*01 TCR.1
YSEHPTFTSQY

(19) United States
(12) Patent Application Publication
(10) Pub. No.: US 2019/0214108 A1
(43) Pub. Date: Jul. 11, 2019

(54) IMMUNOLOGICAL ENTITY CLUSTERING SOFTWARE

(71) Applicant: OSAKA UNIVERSITY, Osaka (JP)

(72) Inventors: Daron Michaelangelo Standley, Osaka (JP); John David Oakley Nieri, Osaka (JP); Songling Li, Osaka (JP); Dimitri Schritt, Osaka (JP); Kazuo Yamashita, Osaka (JP)

(21) Appl. No.: 16/333,875

(22) PCT Filed: Sep. 15, 2017
(86) PCT No.: PCT/JP2017/033530

§ 371 Date: Mar. 15, 2019

(30) Foreign Application Priority Data
Sep. 16, 2016 (JP) 2016-181520

(51) Int. Cl. G16B 15/20; G16B 40/00; G16B 50/00; G16B 45/00

(52) U.S. Cl. CPC G16B 15/20; G16B 45/00; G16B 50/00; G16B 40/00

(57) ABSTRACT

The present invention provides a novel method for classifying antibodies. Specifically, the present invention provides, for a first immunological entity and a second immunological entity, a method for classifying whether a binding epitope is the same or different, and a method for performing clustering based on the classification, the methods including: identifying an array of immunological entities such as antibodies as several portions (for example, a framework region and three CDRs); in order to define a storage region, using the array as a three-dimensional structure model; introducing an index of similarity such as structure and/or array characteristic amounts into an evaluation function for evaluating the similarity or dissimilarity of two immunological entities; and analogizing the similarity of an epitope on the basis of the similarity of an antibody.

### TECHNICAL FIELD

The present invention relates to a method for classifying an immunological entity such as an antibody based on an epitope, production of an epitope cluster, and application thereof.

### BACKGROUND ART

Antibodies are proteins that bind specifically and with high affinity to antigens. A human antibody consists of two macromolecular sequences called a heavy chain and a light chain (FIG. 1). Each of the heavy chain and light chain is further divided into two regions called a variable region and a constant region (FIG. 2). It is also known that such a variable region brings out diversity, which is important for the physiological activity of antibodies. Such a variable region is further decomposed into framework regions and complementarity-determining regions (CDR) (FIG. 3). A molecule to which an antibody binds as a target is referred to as an antigen. An antibody generally binds specifically or with high affinity to an antigen by a CDR physically interacting with an antigen. The region in an antigen that physically interacts with an antibody is called an “epitope” (FIG. 4).

Antibodies are highly diverse. Each individual can create 10¹¹ antibodies with different amino acid sequences. With this diversity, a B cell repertoire can bind to diverse antigens, and with different affinities to different epitopes of the same antigen. The amino acid sequence of the CDR region is the source of diversity. The third loop of a heavy chain (CDR-H3) is the most diverse among CDRs. Multiple antibodies with very different amino acid sequences can bind to the same or very similar epitopes in some cases. With such “sequence degeneration”, it is very difficult to compare antibodies, especially antibodies produced by different individuals, by an antigen or epitope.

Antibodies are highly commercially variable molecules. Many of the most commercially successful drugs today are antibody drugs. Antibody drug is also the field that is growing most rapidly in the pharmaceutical industry. Antibodies are broadly utilized not only for pharmaceutical industries, but also in industries other than basic research and drug development for their high affinity and specificity.

T cells also express receptors (TCR), which are structurally very similar to B cells. An important difference is that TCRs are not soluble and are always bound to a T cell (B cells produce an antibody that is a soluble receptor, and a BCR bound to a cell membrane). While not as diverse as BCRs, T cells also have been studied very extensively. In particular, cell disruption by cytotoxic T cells is important in the action against malignant tumor.

In recent years, next-generation sequencing technologies have enabled large scale identification of the amino acid sequences of antibodies or TCRs. Meanwhile, identification of antigens and epitopes that bind to such antibodies or TCRs is a problem yet to be solved, which is expected to have significant commercial demand.

Existing antigen identification methods are method for experimentally identifying interaction by having an antibody or TCR interact with one or more antigen candidates (e.g., surface plasmon resonance). Alternative technologies thereof include protein chips and various library methods. Such technologies are relatively low cost and high speed, but cannot be applied to proteins or peptides that have been modified after translation, which are important in some diseases such rheumatoid arthritis.

Further, identification of structural epitopes is challenging.

These experimental screening technologies require that the antigen is identified. In other words, an antigen must be identified before the discovery of an antibody or TCR.

Non Patent Literature 1 discloses a calculation method for predicting an antibody specific B cell epitope using residue pairing preferences and cross-blocking.

### CITATION LIST

### Non Patent Literature

[NPL 1] Sela-Culang I. et al., Structure 22, 646-657, 2014

### SUMMARY OF INVENTION

### Solution to Problem

In one aspect, the present invention describes an algorithm for grouping (clustering) immunological entities such as antibodies targeting the same epitope by using only the amino acid sequence information thereof, and an invention utilizing the algorithm. Since BCRs and TCRs are part of the same protein superfamily as antibodies, the methodology in the present invention can be applied to other immunological entities such as BCRs and TCRs. Unlike existing sequence clustering methodologies, the methodology of the inventors uses a three-dimensional model of immunological entities such as antibodies as a feature for grouping sequences of the immunological entities such as antibodies. This methodology has several novel aspects, including: 1. separating a sequence of an immunological entity such as an antibody into several parts (e.g., conserved regions such as framework regions and non-conserved regions such as three CDRs; 2. using a predicted three-dimensional structure model and a sequence to define the conserved regions such as framework regions and non-conserved regions such as CDRs; 3. incorporating parameters such as the structure and sequence features into an evaluation function for evaluating similarity and dissimilarity of two immunological entities such as antibodies; and 4. estimating the similarity of an epitope from similarity of the immunological entities such as antibodies.

The lack of need to identify an immunological entity binder such as an antigen prior to finding a TCR is an important advantage of the clustering algorithm of the invention. The technology of the invention does not require prior knowledge of an immunological entity binder such as an antigen. One of the fascinating applications of the technology of the invention is in use of an antibody or TCR cluster for identification of a drug development target candidate or a biomarker of a disease, an antibody drug, or for genetically modified T cell therapy as chimeric antigen receptor. For example, it is known that BCRs and TCRs exhibit a typical sequence pattern in a certain type of leukemia or lymphoma, so that identification thereof can be used in diagnosis of diseases without knowing the immunological entity binder such as an antigen.

For example, the present invention provides the following.

(1) A method for classifying whether a first immunological entity and a second immunological entity are identical or different for an epitope to be bound thereby, the method comprising the steps of:
(A) identifying conserved regions of amino acid sequences of the first immunological entity and the second immunological entity;
(B) producing three-dimensional structure models of the first immunological entity and the second immunological entity;
(C) superimposing the conserved regions of the first immunological entity and the conserved regions of the second immunological entity in the three-dimensional structure models;
(D) determining similarity between non-conserved regions of the first immunological entity and non-conserved regions of the second immunological entity in the three-dimensional structure models after the superimposition; and
(E) judging whether an epitope binding to the first immunological entity and an epitope binding to the second immunological entity are identical or different based on the similarity.
(1A) The method of item 1, wherein the conserved region comprises a framework region or a part thereof, and the non-conserved regions comprise a complementarity-determining region (CDR) or a part thereof.
(1B) The method of item 1 or 1A, wherein the conserved region of the first immunological entity has a corresponding relationship to the conserved region of the second immunological entity.
(2) The method of item 1, 1A or 1B, wherein the immunological entity is an antibody, an antigen binding fragment of an antibody, a B cell receptor, a fragment of a B cell receptor, a T cell receptor, a fragment of a T cell receptor, a chimeric antigen receptor (CAR), or a cell comprising any one or more of them.
(3) The method of item 1, 1A, 1B, or 2, wherein the conserved regions are identified based on a numbering scheme selected from the group consisting of Kabat, Chotia, modified Chotia, IMGT, and Honnegger.
(4) The method of item 1, 1A, 1B, 2, or 3, wherein the three-dimensional structure models are modeled by a modeling methodology selected from the group consisting of homology modeling, molecular dynamics calculation, fragment assembly, Monte Carlo simulation, energy minimization (simulated annealing or the like), and a combination thereof.
(5) The method of any one of items 1, 1A, 1B, and 2 to 4, wherein the superimposing is performed based on a methodology selected from the group consisting of a least squares method, matrix diagonalization, minimization of root mean square deviation using singular value decomposition, and optimization of structural similarity score based on dynamic programming.
(6) The method of any one of items 1, 1A, 1B, and 2 to 5, wherein the superimposing is performed with an error of one angstrom or less.
(7) The method of any one of items 1, 1A, 1B, and 2 to 6, wherein identical residues are defined in determining the similarity.
(8) The method of item 7, wherein the identical residues are defined based on alignment.
(9) The method of item 8, wherein the alignment comprises the steps of:
A) calculating a structural similarity matrix of all amino acid residues of a given CDR pair; and
B) aligning based on dynamic programming;

wherein if coordinates of two CDRs of the CDR pair are represented by r₁ and r₂, similarity Sₖₗ of any two residues k and l is defined by

$$\begin{matrix}[ Numeral 1 ] & \\ {S}_{kl} = {{e}^{- ( \frac{{r}_{1} [ k ] - {r}_{2} [ l ]}{{d}_{0}} )}}^{2} , & ( 1 )\end{matrix}$$

wherein coordinates of k and l are represented as r₁ and r₂, respectively, and

r₁[i]−r₂[j] [Numerical 2]

is a vector consisting of a difference between coordinates of two amino acids, and d₀ is an empirically determined parameter.
(10) The method of item 9, wherein a C_{α} atom or a center-of-mass coordinate is used as the coordinates.
(11) The method of any one of items 1, 1A, 1B, and 2 to 10, wherein a methodology for expressing the similarity comprises:
(A) calculating a value of

$$[ Numeral 3 ]$$

$${S}_{kl}^{′} = \frac{a}{b + {( {r}_{1} [ k ] - {r}_{2} [ l ] )}^{2}} .$$

wherein a large value indicates a large superimposition; and/or
(B) calculating alignment of amino acids using a global sequence alignment methodology.
(12) The method of any one of items 1, 1A, 1B, and 2 to 11, wherein the similarity is determined based on at least one of a difference in lengths, sequence similarity, and three-dimensional structural similarity.
(13) The method of any one of items 1, 1A, 1B, and 2 to 12, wherein the similarity comprises at least three-dimensional structural similarity.
(14) The method of any one of items 1, 1A, 1B, and 2 to 13, wherein the similarity is selected from the group consisting of a regressive scheme, a neural network method, and machine learning algorithms such as support vector machine and random forest.
(15) A program for making a computer execute the method of any one of items 1, 1A, 1B, and 2 to 14.
(16) A recording medium storing a program for making a computer execute the method of any one of items 1, 1A, 1B, and 2 to 14.
(17) A system comprising a program for making a computer execute the method of any one of items 1, 1A, 1B, and 2 to 14.
(18) An epitope or immunological entity binder (e.g., antigen) having a structure identified by the method of any one of items 1, 1A, 1B, and 2 to 14.
(19) The method of any one of items 1, 1A, 1B, and 2 to 14, comprising the step comprising associating the epitope with biological information.
(19A) The method of any one of items 1, 1A, 1B, 2 to 14 and 19, further comprising the step of identifying the classified epitope.
(19B) The method of item 19A, wherein the identifying comprises at least one selected from the group consisting of determining an amino acid sequence, identifying a three-dimensional structure, identifying a structure other than a three-dimensional structure, and identifying a biological function.
(19C) The method of item 19A or 19B, wherein the identifying comprises determining a structure of the epitope.
(20) A method for generating a cluster of epitopes, comprising the step of classifying immunological entities binding to the identical epitope to the identical cluster using the classification method of any one of items 1, 1A, 1B, 2 to 14, 19, 19A, 19B, and 19C.
(20A) The method of item 20, wherein the immunological entities are evaluated by at least one endpoint selected from the group consisting of a property and similarity with a known immunological entity thereof to perform the cluster classification targeting an immunological entity meeting a predetermined baseline.
(20B) The method of item 20 or 20A, wherein three-dimensional structures of the epitopes are determined to at least partially overlap when a plurality of the epitopes are identical.
(20C) The method of item 20, 20A, or 20B, wherein amino acid sequences of the epitopes are determined to at least partially overlap when a plurality of the epitopes are identical.
(21) A method for identifying a disease, disorder, or biological condition, comprising the step of associating a carrier of the immunological entity with a known disease, disorder, or biological condition based on a cluster generated by the method of item 20, 20A, 20B, or 20C.
(21A) A method for identifying a disease, disorder, or biological condition, comprising the step of evaluating a disease, disorder, or biological condition of a carrier of one or more clusters generated by the method of item 20, 20A, 20B, or 20C by using the cluster.
(21B) The method of item 21A, wherein the evaluating is performed using at least one indicator selected from the group consisting of analysis based on a ranking of quantity and/or a ratio of abundance of the plurality of clusters, and analysis studying a certain number of B cells and quantifying whether there is a cell/cluster similar to a BCR of interest thereamong.
(21C) The method of item 21A or 21B, wherein the evaluating is performed using an indicator other than the cluster.
(21D) The method of item 21C, wherein the indicator other than the cluster comprises at least one selected from the group consisting of a disease associated gene, a polymorphism of a disease associated gene, an expression profile of a disease associated gene, epigenetics analysis, and a combination of TCR and BCR clusters.
(21E) The method of any one of items 21, 21A, 21B, 21C, and 21D, wherein identification of the disease, disorder, or biological condition comprises at least one selected from the group consisting of diagnosis, prognosis, pharmacodynamics, and prediction of the disease, disorder, or biological condition, determination of an alternative method, identification of a patient group, safety evaluation, toxicological evaluation, and monitoring thereof.
(21F) A method for evaluating a biomarker, comprising the step of evaluating the biomarker used as an indicator of a disease, disorder, or biological condition using one or more of epitopes identified by the method of item 19 and/or clusters generated by the method of item 20.
(21G) A method for identifying a biomarker, comprising the step of determining the biomarker or association with a disease, disorder, or biological condition using one or more of epitopes identified by the method of item 19, 19A, 19B, or 19C and/or clusters generated by the method of item 20, 20A, 20B, or 20C.
(22) A composition for identifying the biological information, comprising an immunological entity to an epitope identified based on item 21, 21A, 21B, or 21C.
(22A) A composition for identifying the biological information, comprising an epitope or an immunological entity binder (e.g., antigen) comprising the epitope identified based on item 21, 21A, 21B, or 21C.
(23) A composition for diagnosing the disease, disorder, or biological condition of item 21, comprising an immunological entity to an epitope identified based on item 1.
(23A) A composition for diagnosing the disease, disorder, or biological condition of item 21, comprising a substance targeting an immunological entity to an epitope identified based on items 21, 21A, 21B, or 21C.
(23B) A composition for diagnosing the disease, disorder, or biological condition of item 21, comprising an epitope or an immunological entity binder (e.g., antigen) comprising the epitope identified based on item 21, 21A, 21B, or 21C.
(24) A composition for treating or preventing the disease, disorder, or biological condition of item 21, comprising an immunological entity to an epitope identified based on the method of any one of items 1, 1A, 1B, 2 to 14, 19, 19A, 19B, and 19C.
(24A) The composition of any one of items 22, 22A, 23, 23A, 23B and 24, wherein the immunological entity is selected from the group consisting of an antibody, an antigen binding fragment of an antibody, a T cell receptor, a fragment of a T cell receptor, a B cell receptor, a fragment of a B cell receptor, a chimeric antigen receptor (CAR), and a cell comprising one or more of them (e.g., T cell comprising a chimeric antigen receptor (CAR)).
(24B) A composition for treating or preventing the disease, disorder, or biological condition of item 21, comprising a substance targeting an immunological entity to an epitope identified based on items 21.
(24C) A composition for treating or preventing the disease, disorder, or biological condition of item 21, comprising an epitope or an immunological entity binder (e.g., antigen) comprising the epitope identified based on item 21.
(25) The composition of item 24, wherein the composition comprises a vaccine.
(25A) A composition for evaluating a vaccine for treating or preventing a disease, disorder, or biological condition, comprising an immunological entity to an epitope identified based on item 21.
(26) A computer program for making a computer execute a method for classifying whether a first immunological entity and a second immunological entity are identical or different for an epitope to be bound thereby, the method comprising the steps of:
(A) identifying conserved regions of amino acid sequences of the first immunological entity and the second immunological entity;
(B) producing three-dimensional structure models of the first immunological entity and the second immunological entity;
(C) superimposing the conserved regions of the first immunological entity and the conserved regions of the second immunological entity in the three-dimensional structure models;
(D) determining similarity between non-conserved regions of the first immunological entity and non-conserved regions of the second immunological entity in the three-dimensional structure models after the superimposition; and
(E) judging whether an epitope binding to the first immunological entity and an epitope binding to the second immunological entity are identical or different based on the similarity.
(26A) The program of item 26, further comprising one or more features of the preceding items.
(27) A recording medium storing a computer program for making a computer execute a method for classifying whether a first immunological entity and a second immunological entity are identical or different for an epitope to be bound thereby, the method comprising the steps of:
(A) identifying conserved regions of amino acid sequences of the first immunological entity and the second immunological entity;
(B) producing three-dimensional structure models of the first immunological entity and the second immunological entity;
(C) superimposing the conserved regions of the first immunological entity and the conserved regions of the second immunological entity in the three-dimensional structure models;
(D) determining similarity between non-conserved regions of the first immunological entity and non-conserved regions of the second immunological entity in the three-dimensional structure models after the superimposition; and
(E) judging whether an epitope binding to the first immunological entity and an epitope binding to the second immunological entity are identical or different based on the similarity.
(27A) The recording medium of item 27, further comprising one or more features of the preceding items.
(28) A system for classifying whether a first immunological entity and a second immunological entity are identical or different for an epitope to be bound thereby, the system comprising:
(A) a conserved region identifying unit for identifying conserved regions of amino acid sequences of the first immunological entity and the second immunological entity;
(B) a three-dimensional structure model producing unit for producing three-dimensional structure models of the first immunological entity and the second immunological entity;
(C) a superimposing unit for superimposing the conserved regions of the first immunological entity and the conserved regions of the second immunological entity in the three-dimensional structure models;
(D) a similarity determining unit for determining similarity between non-conserved regions of the first immunological entity and non-conserved regions of the second immunological entity in the three-dimensional structure models after the superimposition; and
(E) an identity judging unit for judging whether an epitope binding to the first immunological entity and an epitope binding to the second immunological entity are identical or different based on the similarity.
(28A) The system of item 28, further comprising one or more features of the preceding items.

The present invention is intended so that one or more of the aforementioned features can be provided not only as the explicitly disclosed combinations, but also as other combinations. Additional embodiments and advantages of the present invention are recognized by those skilled in the art by reading and understanding the following detailed description, as needed.

### Advantageous Effects of Invention

Clustering of antibodies or TCRs by epitope yields an actual significant effect. In particular, clusters classified by each immunological entity binder (e.g., antigen) or epitope are themselves valuable, even if an immunological entity binder (e.g., antigen) is not identified. Such clustering has some direct advantages. For example, this enables comparison of antibody or TCR repertoire from different individuals (e.g., donor X, compared to donor Y, has more expression of cluster Z). Further, the potential for discovery of a disease specific, novel immunological entity binder (e.g., antigen) or epitope and discovery of a novel immunological entity binder (e.g., antigen) are extremely valuable in drug development. In addition, quantitative evaluation of an antibody to an epitope of interest, or more quantitative and high resolution/highly accurate information is obtained in combination with an existing protein chip. Moreover, downstream analysis can be facilitated and reduce cost. For example, instead of screening N BCRs or TCRs, if N receptors are contained in M clusters (N>M), analysis can be completed by M rounds of screenings. Furthermore, one feature of clustering is that clustering can be a technology that is complementary to experimental screening, such as virtual screening (estimation of an immunological entity binder (e.g., antigen) or epitope by similarity search) using BCRs or TCRs with a known immunological entity binder (e.g., antigen) or epitope.

Since antibodies with different amino acid sequences can recognize the same epitope, conventional bioinformatics tools such as sequence alignment are not methodologies that are appropriate for clustering of antibodies by epitope. While bioinformatics have docking for predicting the so-called protein complex structure, and methodologies that predict the complex structure based on similarity to the interface of a known protein complex, these are also not methodologies that are suitable for clustering of antibodies by epitope. TCRs also have a similar problem, but the problem is further complicated in that an immunological entity binder (e.g., antigen) is a complex of a one-dimensional peptide and an MHC which is a molecule presenting the peptide, where MHCs are themselves diverse. Therefore, the invention is important in that conventional methodologies are not able to cluster antibodies or TCRs by epitope with a robust scheme.

### BRIEF DESCRIPTION OF DRAWINGS

FIG. 1 depicts a typical schematic diagram of a human antibody. The left panel emulates a heavy chain and a light chain, and the structure on the right side depicts how heavy chains and light chains are structured. The left side depicts a schematic diagram at the sequence level, and the right side depicts a schematic diagram at the structural level.

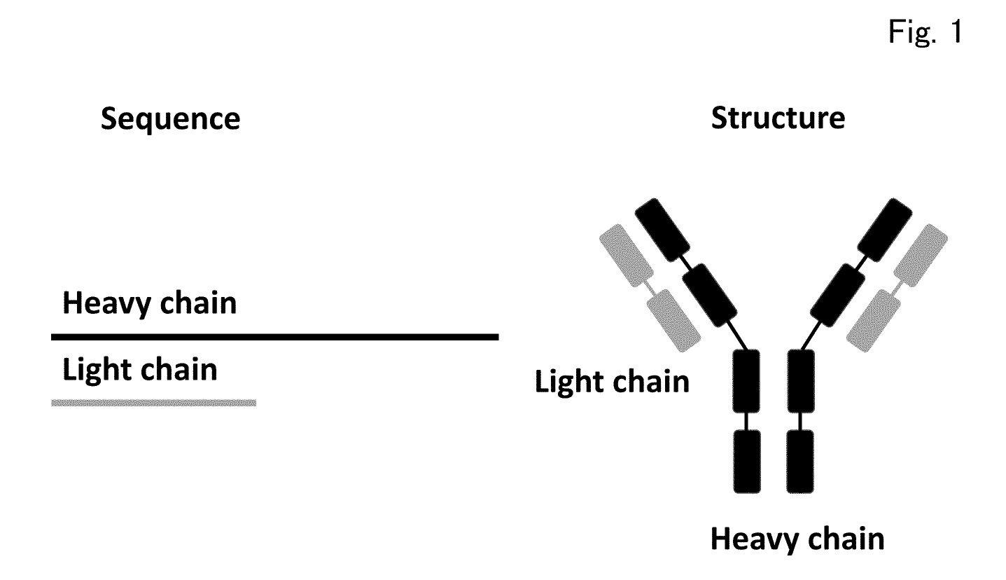

FIG. 2 is a schematic diagram further dividing heavy chains and light chains into regions. Each of the heavy chains and light chains is further divided into two regions, i.e., variable region and constant region. The left side depicts a schematic diagram at the sequence level, and the right side depicts a schematic diagram at the structure level.

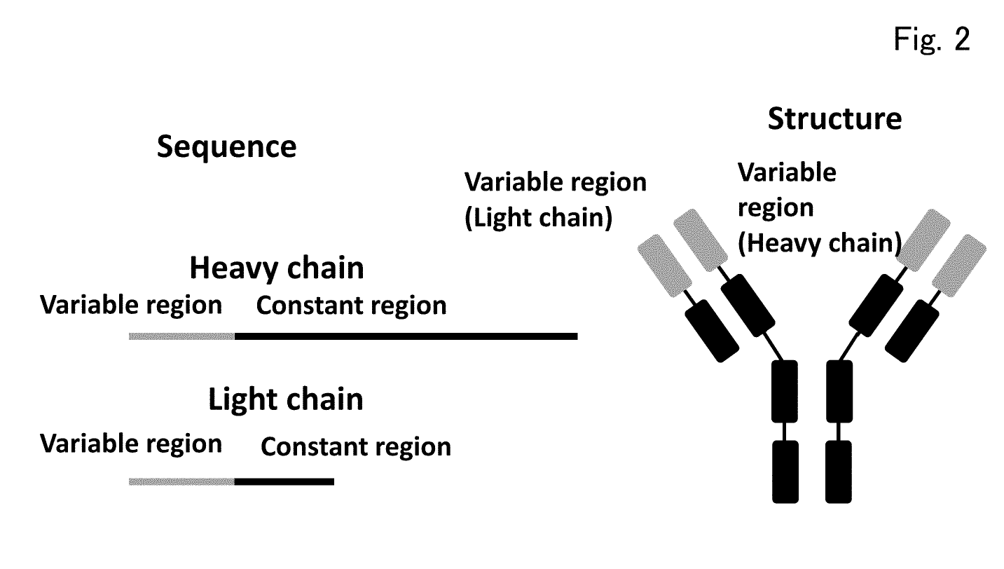

FIG. 3 is a diagram further explaining a variable region. A variable region is further separated into a conserved region such as a framework region and a non-conserved region such as a complementarity-determining region (CDR), which is further divided into CDR1, CDR2, and CDR3. The definition of the status is the following. 1 to 3: non-conserved regions (e.g., CDR1 to 3), 4: conserved regions (e.g., framework region), and 0: others.

FIG. 4 is a schematic diagram of an epitope, which is a region that physically interacts with an antibody in an antigen.

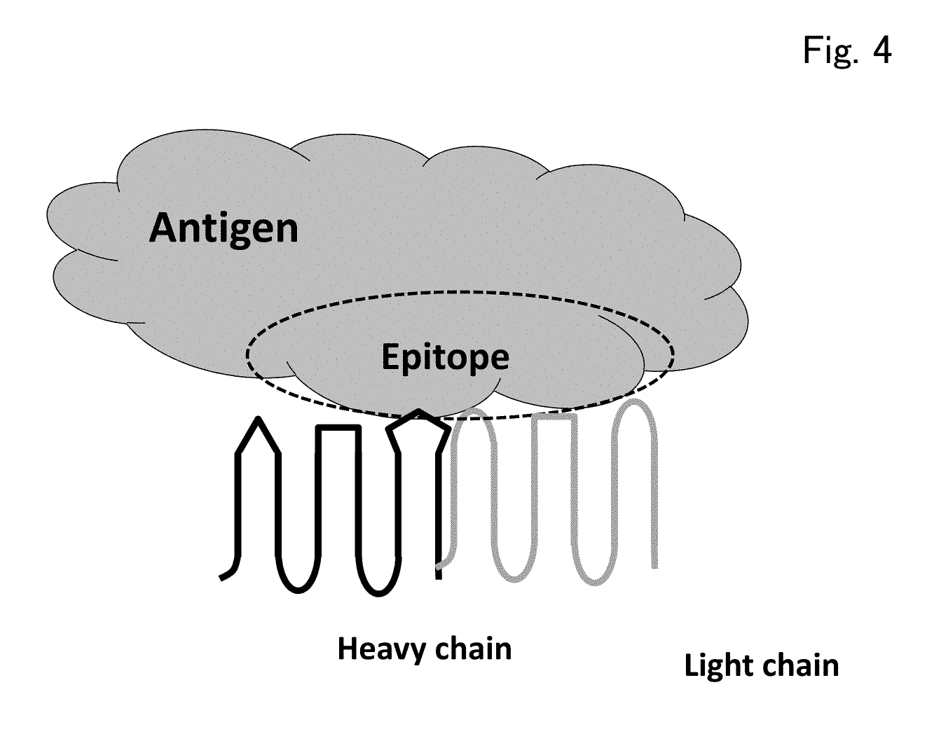

FIG. 5 depicts a schematic diagram of a CDR, which is an example of a non-conserved region, and depicts structure 1 on the left and structure 2 on the right in the top panel. The left side of the bottom panel depicts a schematic diagram superimposing the frameworks of structure 1 and structure 2 as an example of a conserved region. The right side of the bottom panel set forth the definition of an equivalent residue. (1, 1), (2, 2), (3, -), (4, 3), (6, -), and (7, 5) are depicted in this figure. A structure similarity matrix is depicted under the arrow in the bottom panel.

FIG. 6A depicts an antibody superimposed with an antigen (example of HIV Env protein).

FIG. 6B depicts a typical diagram of an antibody network.

FIG. 7 shows classification of HIV and non-HIV in a training set using the KOTAI program (using a predicted structure) which is an example of the invention in the top graph. HIV is shown on the left side (dark gray) and non-HIV is shown on the right side (light gray). The bottom graph shows classification of HIV and non-HIV in a training set using the conventional BLAST program (which does not use a predicted structure). Specifically, a feature is used for learning of support vector machine (SVM). SVM evaluates in the following manner using 5-fold cross validation: 1) all possible anti-HIV antibody pairs (to the same or different epitopes) are randomly divided into a learning set and a validation set; 2) SVM learns to distinguish anti-HIV antibodies recognizing the same epitope (positive) from antibodies recognizing different epitopes (negative) to validate the performance using the validation set; and 3) the experiment discussed in Example 1 is conducted. FIG. 7 shows the result thereof.

FIG. 8 shows a result of outputting a distance matrix of each pair by SVM, and the accuracy when using the present invention. Both panels show results of clustering all anti-HIV antibodies using a distance matrix at the end. The results are evaluated by the similarity to the actual network. The results are shown with a network created with sequence similarity (similarity from alignment obtained by the program BLAST) which is a conventional art. FIG. 8A shows the accuracy of the proposed algorithmic epitope network using the present invention. The accuracy (Adjusted Rand index) was computed as 0.72. The accuracy computed using the BLAST network was computed as 0 in FIG. 8B.

FIG. 8 shows a result of outputting a distance matrix of each pair by SVM, and the accuracy when using the present invention. Both panels show results of clustering all anti-HIV antibodies using a distance matrix at the end. The results are evaluated by the similarity to the actual network. The results are shown with a network created with sequence similarity (similarity from alignment obtained by the program BLAST) which is a conventional art. FIG. 8A shows the accuracy of the proposed algorithmic epitope network using the present invention. The accuracy (Adjusted Rand index) was computed as 0.72. The accuracy computed using the BLAST network was computed as 0 in FIG. 8B.

FIG. 9 shows the result of clustering anti-HIV antibodies and non-anti-HIV antibodies with a distance matrix obtained by SVM for a consolidated set of anti-HIV and non-anti-HIV antibodies. The accuracy using the present invention is shown. FIG. 9A shows the accuracy of the proposed algorithmic epitope network for anti-HIV antibodies using the present invention. The accuracy (Adjusted Rand index) was computed as 0.82. The accuracy computed using the BLAST network was computed as 0 for non-anti-HIV antibodies in FIG. 9B.

FIG. 9 shows the result of clustering anti-HIV antibodies and non-anti-HIV antibodies with a distance matrix obtained by SVM for a consolidated set of anti-HIV and non-anti-HIV antibodies. The accuracy using the present invention is shown. FIG. 9A shows the accuracy of the proposed algorithmic epitope network for anti-HIV antibodies using the present invention. The accuracy (Adjusted Rand index) was computed as 0.82. The accuracy computed using the BLAST network was computed as 0 for non-anti-HIV antibodies in FIG. 9B.

FIG. 10 is a schematic diagram of the configuration of the system of the invention.

FIG. 11 is a schematic flow of the present invention.

FIG. 12 shows an epitope sequence (CMV TCR data) used in Example 5.

FIG. 13 shows the results in Example 5 (CMV specific TCR clustering). The kernel function was “rbf”, and class_weigh option was “balanced”. The results were obtained by using a threshold value of 0.34 and separating TCR pairs into two classes (pair distance is <0.34 (left) and >=0.34 (right)), and evaluating whether TCR pairs belonging to each class recognize identical epitopes.

FIG. 14 depicts a schematic diagram of two types of anti-hemagglutinin BCRs in PDB.

FIG. 15 depicts an experimental design for obtaining anti-stem BCRs and anti-non-stem BCRs.

FIG. 16 shows the procedure (analysis method) of the 3D modeling phase and clustering phase of a method for analyzing sequence data.

FIG. 17 shows the distribution of StrucSim values for a known anti-HA PDB entry (FIG. 17A) and 77 anti-HA mouse BCRs (FIG. 17B).

FIG. 18 shows the cutoff (structural characteristic, StrucSim>=0.95) for separating stem and non-stem classes to different epitopes. The X axis indicates the evaluation value, and the Y axis indicates the frequency. A strict cutoff was selected after analyzing the character distribution within a model.

FIG. 19 shows stem (triangle) and non-stem (circle) clusters, made visible using the Python NetworkX graphviz package. Bound BCRs were sufficiently separated with the proposed characteristic.

### DESCRIPTION OF EMBODIMENTS

The present invention is explained hereinafter with the best modes thereof. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present invention pertains. In case of a contradiction, the present specification (including the definitions) takes precedence.

### Definition

The definitions of the terms and/or the detailed basic technology that are particularly used herein are explained hereinafter as appropriate.

As used herein, “immunological entity” refers to any substance responsible for an immune reaction. Immunological entities include antibodies, antigen binding fragments of an antibody, T cell receptors, fragments of a T cell receptor, B cell receptors, fragments of a B cell receptor, chimeric antigen receptors (CAR), cells comprising one or more of them (e.g., T cells comprising a chimeric antigen receptor (CAR) (CAR-T)), and the like. Immunological entities can be broad, similarly including immunologically related entities used in analysis of a phage display or the like (including scFv and nanobodies) artificially imparted with diversity and nanobodies produced by an animal such as alpaca. As used herein, descriptions of “first”, “second”, etc. (“third” . . . and the like) indicate that entities are different from each other.

As used herein, “antibody” is used in the same meaning that is commonly used in the art and refers to a protein reacting highly specifically to an antigen, which is made in the immune system when the antigen contacts the biological immune system (antigen stimulation). Each of the antibodies to an epitope used in the present invention may be of any origin, type, shape, or the like, as long as the antibody binds to the specific epitope. The antibodies described herein can be divided into framework regions and antigen binding regions (CDR).

As used herein, “T cell receptor (TCR)” is also called a T cell antigen receptor. A T cell receptor refers to a receptor recognizing an antigen, expressed on a cell membrane of a T cell that plays a central role in the immune system. TCRs have an α chain, β chain, γ chain, and δ chain, with which an αβ or γδ dimer is constituted. TCRs consisting of the combination of the former are called αβ TCRs, and TCRs consisting of the combination of the latter are called γδ TCRs. T cells having such TCRs are respectively called αβ T cells and γδ T cells. The TCRs are structurally very similar to a Fab fragment of an antibody produced by B cells and recognize antigen molecules bound to an MHC molecule. Since a TCR gene of a mature T cell has undergone gene rearrangement, an individual has highly diverse TCRs that enable recognition of various antigens. TCRs also form a complex by binding to a non-variable CD3 molecule at the cell membrane. CD3 has an amino acid sequence called ITAM (immunoreceptor tyrosine-based activation motif) in the intracellular region. This motif is considered to be involved in intracellular signaling. Each TCR chain is comprised of a variable domain (V) and a constant domain (C). A constant domain has a short cytoplasm section penetrating the cell membrane. A variable domain is present outside the cell and binds to an antigen-MHC complex. A variable domain has three hypervariable domains or regions called complementarity-determining regions (CDRs), which bind to an antigen-MHC complex. The three CDRs are called CDR1, CDR2, and CDR3. TCR gene rearrangement is similar to the process of B cell receptors known as immunoglobulins. For gene rearrangement of αβ TCRs, VDJ recombination of β chain is performed, followed by VJ recombination of an α chain. When the α chain is rearranged, the gene of the δ chain is deleted from the chromosome. Thus, a T cell having an αβ TCR would never have a γδ TCR simultaneously. In contrast, a signal via a γδ TCR in a T cell having the TCR suppresses the expression of β chain, so that a T cell having a γδ TCR would never have an αβ TCR simultaneously.

As used herein, “B cell receptor (BCR)” is also called a B cell antigen receptor, referring to those comprised of Igα/Igβ (CD79a/CD79b) heterodimer (α/β) associated with a membrane bound immunoglobulin (mIg) molecule. An mIg subunit binds to an antigen to induce aggregation of receptors, while an α/β subunit transmits a signal toward the cell. Aggregation of BCRs is understood to quickly activate Lyn, Blk, and Fyn of an Src family kinase in the same manner as Syk and Btk of tyrosine kinase. Many different results are produced depending on the complexity of BCR signaling. Examples thereof include survival, resistance (allergy; lack of hypersensitive reaction to an antigen) or apoptosis, cell division, differentiation into an antibody producing cell or memory B cell, and the like. Many hundreds of million types of T cells with difference sequences of the variable regions of TCRs are produced, and many hundreds of million types of B cells with difference sequences of the variable regions of BCRs (or antibodies) are produced. Since the individual sequences of TCRs and BCRs vary due to rearrangement or mutation of the genomic sequence, a clue for antigen specificity of a T cell or B cell can be found by determining the sequence of mRNA (cDNA) or the genomic sequence of TCR/BCR.

As used herein, “chimeric antigen receptor (CAR)” is a collective term for chimeric proteins having a single chain antibody (scFv) having a light chain (VL) and a heavy chain (VH) of a tumor antigen specific monoclonal antibody variable region bound in series on the N-terminus side, and a T cell receptor (TCR) ζ chain on the C-terminus side. A chimeric antigen receptor is an artificial T cell receptor used in gene and cell therapy, in which an artificial T cell receptor that is genetically engineered to defeat the immune evasion mechanism of tumor is transfected into patient T cells, which are amplified and cultured outside the body and then injected into a patient (Dotti G, et al., Hum Gene Ther 20: 1229-1239, 2009). Such a CAR can be produced using an epitope that is identified or clustered by the present invention. Gene and cell therapy can be materialized using the produced CAR or genetically modified T cells comprising such a CAR (see Credit: Brentjens R, et al. “Driving CAR T cells forward.” Nat Rev Clin Oncol. 2016 13, 370-383 and the like).

As used herein, “gene region” refers to a framework region, antigen binding region (CDR), and each of the regions such as the V region, D region, J region, and C region. Such gene regions are known in the art and can be appropriately determined by referring to a database or the like. As used herein, “homology” of genes refers to the degree of identity of two or more gene sequences to one another. Generally, having “homology” refers to having a high degree of identity or similarity. Therefore, two genes having higher homology have higher identity or similarity of the sequences thereof. Whether two genes have homology can be found by direct comparison of sequences, or by hybridization under stringent conditions for nucleic acids. As used herein, “homology search” refers to a search for homology. Preferably, homology can be searched in silico using a computer.

As used herein, “V region” refers to a variable domain (V) region of a variable region of an immunological entity such as an antibody, TCR, or BCR.

As used herein, “D region” refers to a D region of a variable region of an immunological entity such as an antibody, TCR, or BCR.

As used herein, “J region” refers to a J region of a variable region of an immunological entity such as an antibody, TCR, or BCR.

As used herein, “C region” refers to a constant domain (C) region of an immunological entity such as an antibody, TCR, or BCR.

As used herein, “repertoire of a variable region” refers to a collection of V(D)J regions optionally created by gene rearrangement in TCR or BCR. The phrases TCR repertoire, BCR repertoire and the like are used, but they can also be called, for example, T cell repertoire, B cell repertoire, or the like. For example, “T cell repertoire” refers to a collection of lymphocytes characterized by the expression of a T cell receptor (TCR) serving an important role in antigen recognition or recognition of an immunological entity binder. Since a change in T cell repertoire is a significant indicator of an immune state in a diseased state or physiological state, T cell repertoire analysis has been performed for identification of antigen specific T cells involved in the development of a disease and diagnosis of T lymphocyte abnormalities.

TCRs and BCRs create various gene sequences by gene rearrangement of multiple gene fragments of the V region, D region, J region, and C region on the genome.

As used herein, “isotype” refers to IgM, IgA, IgG, IgE, IgD, and the like, which belong to the same type but have different sequences from one another. Isotypes are denoted using various gene abbreviations and symbols.

As used herein, “subtype” is a type within type in IgA and IgG for BCRs. IgG has IgG1, IgG2, IgG3, and IgG4, and IgA has IgA1 and IgA2. Subtypes are also known to be in β and γ chains for TCRs, having TRBC1 and TRBC2 and TRGC1 and TRGC2, respectively.

As used herein, “immunological entity binder” refers to any substrate that can be specifically bound by an immunological entity such as an antibody, TCR, or BCR. When denoted as “antigen” herein, the antigen can broadly refer to a “immunological entity binder”. “Antigen” can be used narrowly in pair with an antibody and refers narrowly to any substrate that can be specifically bound by an “antibody” in the art.

As used herein, “epitope” refers to a site in a molecule of an immunological entity binder (e.g., antigen), to which an immunological entity such as an antibody or a lymphocyte receptor (TCR, BCR, or the like) binds. While a straight chain of an amino acid can constitute an epitope (strain chain epitope), separated sites of a protein can constitute a stereo structure to function as an epitope (conformational epitope). Epitopes of the invention are not limited by such detailed classification of epitopes. It is understood that if certain immunological entities such as antibodies have the same epitope, an immunological entity such as an antibody having another sequence can also be used in the same manner.

As used herein, whether epitopes are “identical” or “different” can be determined by similarity (amino acid sequence, three-dimensional structure, or the like) in accordance with the classification based on the present invention. “Identical” does not refer to complete identity of amino acid sequences, but refers to substantially the same quality of stereo structure. Epitopes belonging to identical epitope cluster are determined as “identical” in the present invention. Therefore, “different” epitopes refer to epitopes that do not belong to the “identical” cluster. In one embodiment, it can be determined whether they belong to identical cluster depending on whether the epitopes are “identical” or “different”. When performing cluster analysis, an epitope is, in comparison to another epitope, determined to be identical if belonging to the same cluster, and determined to be different if belonging to a different cluster. Therefore, immunological entities that bind to identical epitopes can be classified into identical cluster to generate the cluster. Immunological entities can also be evaluated for at least one endpoint selected from the group consisting of properties and similarity with a known immunological entity thereof to perform the cluster classification by targeting an immunological entity meeting a predetermined baseline. Thus in one embodiment, when the epitopes are identical, the three-dimensional structures of the epitopes can at least partially or completely overlap, or the amino acid sequences of the epitopes can at least partially or completely overlap. It is suitable to determine a threshold value as an important indicator to be highly compatible with structural data or the like that can be confirmed with certainty, but other threshold values can be employed when prioritizing statistical significance. Those skilled in the art can determine an appropriate threshold value by referring to the descriptions herein depending on the situation. For example, a pair with a maximum distance found by clustering analysis using a hierarchical clustering methodology (e.g., group average method (average linkage clustering), nearest neighbor method (NN method), K-NN method, Ward method, furthest neighbor method, or centroid method) of less than a specific value can be deemed to be in identical cluster. Examples of such a value include, but are not limited to, less than 1, less than 0.95, less than 0.9, less than 0.85, less than 0.8, less than 0.75, less than 0.7, less than 0.65, less than 0.6, less than 0.55, less than 0.5, less than 0.45, less than 0.4, less than 0.35, less than 0.3, less than 0.25, less than 0.2, less than 0.15, less than 0.1, less than 0.05, and the like. The clustering methodology is not limited to hierarchical methodologies. A non-hierarchical methodology may also be used.

As used herein, “cluster” of epitopes generally refers to elements that are similar among elements of a population (in this case epitopes) collected from a distribution of the elements in a multi-dimensional space without external standards or designation of the number of groups. As used herein, a cluster refers to a collection of similar epitopes among a large number of epitopes. Epitopes belonging to an identical cluster bind to the same antibody. The epitopes can be classified by multivariate analysis. A cluster can be configured using various cluster analysis methodologies. A cluster of epitopes provided by the present invention has been demonstrated to reflect the biological condition (e.g., disease, disorder, or drug efficacy, especially immune state or the like) by showing that an epitope belongs to the cluster.

As used herein, “similarity” refers to the degree molecules are similar for molecules such as an immunological entity binder (e.g., antigen) or epitope or a part thereof. Similarity can be determined based on a difference in lengths, sequence similarity, three-dimensional structural similarity, or the like. Generally, the concept encompasses a broadly defined “structural similarity”. Although not wishing to be bound by any theory, it is understood that antibodies, TCRs, BCRs, or the like binding to an epitope belonging to an identical cluster can be assigned to a disease, disorder, symptom, physiological phenomenon, or the like in the same category when epitopes are classified based on such similarity in some of the embodiments of the present invention. Therefore, a variety of diagnosis (incidence of cancer, compatibility of administered drug, and the like) is made possible by studying whether there are antibodies, TCRs, BCRs, or the like that react to the same epitope cluster by using the methodologies of the invention.

As used herein, “similarity scope” refers to a specific value indicating similarity. This is also referred to as similarity”. A suitable scope can be appropriately employed depending on the technique used in calculating structural similarity. A similarity score can be computed using, for example, a regressive scheme, a neural network method, or a machine learning algorithm such as support vector machine or random forest.

As used herein, “conserved region”, in the context of an immunological entity, refers to a region where a structure is conserved across a plurality of immunological entities. Examples of conserved regions include, but are not limited to, a framework region or a part thereof of an antibody or the like.

As used herein, “non-conserved region”, in the context of an immunological entity, refers to a region where a structure is not conserved across a plurality of immunological entities. Examples of non-conserved regions include, but are not limited to, a complementarity-determining region (CDR) or a part thereof of an antibody or the like.

As used herein, “complementarity-determining region (CDR)” is a region forming a binding site by actually contacting an immunological entity binder (e.g., antigen) in an immunological entity such as an antibody. In general, a CDR is positioned on Fv (including a heavy chain variable region (VH) and light chain variable region (VL)) of an antibody or a molecule corresponding to an antibody (immunological entity). In general, CDRs have CDR1, CDR2, and CDR3 consisting of about 5 to 30 amino acid residues. In addition, it is known that especially heavy chain CDRs contribute to an antibody binding to an antigen in an antigen-antibody reaction. Among the CDRs, CDR3, especially CDR-H3, is known to contribute the most in an antibody binding to an antigen. For example, “Willy et al., Biochemical and Biophysical Research Communications Volume 356, Issue 1, 27 Apr. 2007, Pages 124-128” describes that the binding capability of an antibody was enhanced by modifying a heavy chain CDR3. A plurality of definitions of CDRs and methods for determining the position thereof have been reported. For example, the definition of Kabat (Sequences of Proteins of Immunological Interest, 5th ed., Public Health Service, National Institutes of Health, Bethesda, Md. (1991)) or Chothia (Chothia et al., J. Mol. Biol., 1987; 196: 901-917) may be employed. In one embodiment of the present invention, the definition of Kabat is used as a suitable example, but the definition is not necessarily limited thereto. In some cases, CDRs can be determined by considering both the definition of Kabat and the definition of Chothia (modified Chothia method). For example, a CDR can be the overlapping portion of CDRs according to each definition or a portion comprising both CDRs according to each of the definitions. Alternatively, a CDR can be determined in accordance with IMGT or Honegger. Specific example of such a method includes the method of Martin et al. using Oxford Molecular's AbM antibody modeling software (Proc. Natl. Acad. Sci. USA, 1989; 86: 9268-9272), which is a combination between the definition of Kabat and the definition of Chothia. The present invention can be practiced using information of such CDR. As used herein, “CDR3” refers to the third complementarity-determining region (CDR). Herein, CDR is a region, among the variable region, directly contacting an immunological entity binder (e.g., antigen) with a particularly large variation, and is referred to as a hypervariable region. Each of the variable regions of a light chain and a heavy chain has three CDRs (CDR1 to CDR3) and four FRs (FR1 to FR4) surrounding the three CDRs. Since a CDR3 region is understood to straddle the V region, D region, and the J region, a CDR3 region is considered to be a key for a variable region and is used as a subject of analysis.

As used herein, “framework region” refers to a region of an Fv region other than CDRs. A framework region generally consists of FR1, FR2, FR3, and FR4, and is considered relatively well conserved among antibodies (Kabat et al., “Sequence of Proteins of Immunological Interest” US Dept. Health and Human Services, 1983.) Therefore, the present invention can employ a methodology that immobilizes the framework region when comparing each sequence.

As used herein, “identification” of a region such as an amino acid sequence refers to characterization of the amino acid sequence from a certain viewpoint, and refers to determination of a region by a characteristic having one property. Identification includes, but is not limited to, specifically identifying a region comprising an amino acid number, linking a characteristic related to these regions, and the like. As used herein, “division” of a region such as an amino acid sequence refers to characterizing the amino acid sequence and then distinguishing each region determined by a characteristic having one property into separate regions. Such identification and division can be performed using any technology used in the field of bioinformatics such as Kabat, Chotia, modified Chotia, IMGT, Honegger, or the like. Identification of a conserved region exemplified by a framework or the like when processing a region such as an amino acid sequence is an important characteristic herein. Decomposition into conserved regions and non-conserved regions (e.g., CDR or the like) as a result of identification is also envisioned. When identifying and superimposing parts of conserved regions or non-conserved regions of two or more immunological entities, it is preferable that the parts of immunological entities have a substantially corresponding relationship. As used herein, “corresponding relationship”, in the context of a conserved region, is a relationship in which a part of a first immunological entity and a part of a second immunological entity can be superimposed on each other when considering the position of a three-dimensional structure. For a non-conserved region, amino acid residues corresponding to each other when considering the position of a three-dimensional structure would be present by defining identical residues explained herein. Therefore, “corresponding relationship” can be confirmed by alignment of a sequence or the like, identification of identical residues or the like.

As used herein, “three-dimensional structure model”, in the context of a macromolecule of a protein comprising an immunological entity such as an antibody, refers to a model of a three-dimensional structure (tertiary structure, steric conformation, or conformation) constructed based on the amino acid sequence of the protein or the like. Production of such a model is referred to as modeling. The amino acid sequence of a protein is called a primary structure. In an organism, the primary structure of most proteins uniquely has a three-dimensional structure after undergoing folding. Examples of methodologies for producing a three-dimensional model (modeling) include, but are not limited to, homology modeling, molecular dynamics calculation, fragment assembly, a combination thereof, and the like.

As used herein, “superimpose” (or “superpose”) refers to superimposing a stereo structure of a molecule such as an immunological entity and the stereo structure of a molecule such as another immunological entity. Superimposing can be typically performed by superimposing the position, coordinate, or the like of each atom in the molecules. When superimposing, matrix diagonalization, minimalization of root mean square deviation using singular value decomposition, or the like can be used to superimpose as close as possible. Structures can be superimposed, generally with an error of several angstroms (about 2 Å, about 3 Å, about 4 Å, about 5 Å, about 6 Å, about 7 Å, about 8 Å, about 9 Å, or the like), or of one angstrom in a preferred embodiment.

As used herein, “defining identical residues” refers to determining, when determining the structural similarity after superimposing two immunological entities (e.g., antibodies, TCRs, BCRs, or the like), amino acid residues corresponding to each other structurally, i.e., when considering the position of a three-dimensional structure. Since an amino acid corresponding to an amino acid on one side may not be present in another, such a case is defined as lacking identical residues.

As used herein, “alignment” ((noun) or align (verb)) refers to primary structure of DNA, RNA, or proteins lined up so that a similar region can be identified in bioinformatics. This often provides a hint to find the functional, structural, or evolutionary relationship of sequences. A sequence of aligned amino acid residues or the like is typically expressed as a row in a matrix, and a gap is inserted so that sequences with identical or similar properties are lined up in the same column. When comparing two sequences, this is called a pairwise sequence alignment, which is used when studying the similarity in a part or whole the alignment of two sequences in detail. For alignment, dynamic programming can be typically used. Representative methodologies that can be used include Needleman-Wunsch method for global alignment, and Smith-Waterman method for local alignment. In this regard, global alignment is alignment for all residues in a sequence and is effective for comparison between sequences of approximately the same length. Local alignment is effective when sequences are not similar as a whole, but it is desirable to find partial similarity. As used herein, “mismatch” refers to the presence of a base or amino acid that is not identical to each other when nucleic acid sequences, amino acid sequences of the like are aligned. “Gap” refers to the presence of a base or amino acid that is present in one, but not in the other in an alignment.

As used herein, “assign” refers to assignment of information such as a specific gene name, function, or characteristic region (e.g., V region, J region, or the like) to a sequence (e.g., nucleic acid sequence, protein sequence, or the like). Specifically, assignment can be accomplished by inputting or linking specific information to a sequence.

As used herein, “specific” refers to having low binding capability to, preferably does not bind to, another sequence in at least a pool of target antibodies, TCRs, or BCRs that bind to a target sequence, or preferably in all existing antibody, TCR, or BCR sequences. A specific sequence is advantageously, but not necessarily limited to being, fully complementary to a target sequence.

As used herein, “protein”, “polypeptide”, “oligopeptide” and “peptide” are used herein to have the same meaning and refer to a polymer of amino acids with any length. The polymer may be straight, branched, or cyclic. An amino acid may be a naturally-occurring, non-naturally occurring, or modified amino acid. The term may also encompass those assembled into a complex of multiple polypeptide chains. The term also encompasses naturally-occurring or artificially modified amino acid polymers. Examples of such a modification include disulfide bond formation, glycosylation, lipidation, acetylation, phosphorylation, and any other manipulation or modification (e.g., conjugation with a labeling component). The definition also encompasses, for example, polypeptides comprising one or more analogs of an amino acid (e.g., including non-naturally occurring amino acids and the like), peptide-like compounds (e.g., peptoids), and other known modifications in the art.

As used herein, “amino acid” may be naturally-occurring or non-naturally-occurring amino acids as long as the objective of the present invention is met.

As used herein, “polynucleotide”, “oligonucleotide” and “nucleic acid” are used herein to have the same meaning, and refer to a polymer of nucleotides with any length. The term also encompasses “oligonucleotide derivative” and “polynucleotide derivative”. “Oligonucleotide derivative” and “polynucleotide derivative” refer to an oligonucleotide or polynucleotide that comprises a nucleotide derivative or has a bond between nucleotides which is different from normal. The terms are used interchangeably. Specific examples of such an oligonucleotide include 2′-O-methyl-ribonucleotide, oligonucleotide derivatives having a phosphodiester bond in an oligonucleotide converted to a phosphorothioate bond, oligonucleotide derivatives having a phosphodiester bond in an oligonucleotide converted to an N3′-P5′ phosphoramidate bond, oligonucleotide derivatives having ribose and phosphodiester bond in an oligonucleotide converted to a peptide nucleic acid bond, oligonucleotide derivatives having uracil in an oligonucleotide replaced with C-5 propinyluracil, oligonucleotide derivatives having uracil in an oligonucleotide replaced with C-5 thiazoluracil, oligonucleotide derivatives having cytosine in an oligonucleotide replaced with C-5 propinylcytosine, oligonucleotide derivatives having cytosine in an oligonucleotide replaced with phenoxazine-modified cytosine, oligonucleotide derivatives having ribose in DNA replaced with 2′-O-propylribose, oligonucleotide derivatives having ribose in an oligonucleotide replaced with 2′-methoxyethoxyribose, and the like. Unless noted otherwise, specific nucleic acid sequences are also intended to encompass conservatively modified variants (e.g., degenerate codon substitute) and complement sequences in the same manner as the expressly shown sequences. Specifically, degenerate codon substitutes can be achieved by preparing a sequence with the third position of one or more selected (or all) codons substituted with a mixed base and/or deoxyinosine residue (Batzer et al., Nucleic Acid Res. 19: 5081 (1991); Ohtsuka et al., J. Biol. Chem. 260: 2605-2608 (1985); Rossolini et al., Mol. Cell. Probes 8: 91-98 (1994)). As used herein, “nucleic acid” is used interchangeably with a gene, cDNA, mRNA, an oligonucleotide, and polynucleotide. As used herein, a “nucleotide” may be naturally-occurring or non-naturally occurring.

As used herein, “gene” refers to an agent defining a genetic trait. A gene is generally arranged in a certain order on a chromosome. A gene defining the primary structure of a protein is referred to as a structural gene, and a gene determining the expression thereof is referred to as a regulator gene. As used herein, “gene” may refer to “polynucleotide”, “oligonucleotide”, and “nucleic acid”. A “gene product” is a substance produced based on a gene and refers to a protein, mRNA, or the like.

As used herein, “homology” of genes refers to the degree of identity of two or more genetic sequences with one another. In general, having “homology” refers to having a high degree of identity or similarity. Thus, two genes with higher homology have higher identity or similarity of sequences. It is possible to find whether two types of genes have homology by direct comparison of sequences, or by hybridization under stringent conditions for nucleic acids. When two genetic sequences are directly compared, the genes are homologous when DNA sequences are representatively at least 50% identical, preferably at least 70% identical, and more preferably at least 80%, 90%, 95%, 96%, 97%, 98%, or 99% identical between the genetic sequences. Thus, as used herein, “homolog” or “homologous gene product” refers to a protein in another species, preferably mammal, exerting the same biological function as a protein constituent of a complex which will be further described herein.

Amino acids may be denoted herein by a one character symbol recommended by the IUPAC-IUB Biochemical Nomenclature Commission. Nucleotides may similarly be denoted by a commonly recognized one character code. Comparison of similarity, identity, and homology of an amino acid sequence and a base sequence is computed herein with a default parameter using a sequence analysis tool BLAST. For example, identity can be searched by using BLAST 2.2.28 (published on Apr. 2, 2013) of NCBI. Herein, values for identity generally refer to a value obtained by alignment under the default condition using the aforementioned BLAST. However, when a higher value is obtained by changing a parameter, the highest value is considered the value of identity. When identity is evaluated in multiple regions, the highest value thereamong is considered the value of identity. Similarity is a value taking into consideration similar amino acid in addition to identity into the calculation.

As used herein, a “fragment” refers to a polypeptide or a polynucleotide having a sequence length of 1 to n−1, relative to a full length polypeptide or polynucleotide (of length n). The length of the fragment can be appropriately changed depending on the objective thereof. Examples of the lower limit of the length for a polypeptide include 3, 4, 5, 6, 7, 8, 9, 10, 15, 20, 25, 30, 40, 50 and more amino acids. A length represented by an integer which is not specifically listed herein (e.g., 11 or the like) can also be appropriate as the lower limit. For a polynucleotide, examples of the lower limit include 5, 6, 7, 8, 9, 10, 15, 20, 25, 30, 40, 50, 75, 100 and more nucleotides. A length represented by an integer which is not specifically listed herein (e.g., 11 or the like) can also be appropriate as the lower limit. For such a fragment as used herein, it is understood, for example, that when a full length polypeptide or polynucleotide functions as a marker, the fragment itself is also within the scope of the present invention as long as it functions as a marker.

A functional equivalent, such as an isotype, of a molecule such as IgG used in the present invention can be found by searching a database or the like. As used herein, “search” refers to finding another nucleic acid base sequence having a specific function and/or property by utilizing a certain nucleic acid base sequence with electronic, biological, or other methods, and preferably electronic methods. Electronic search includes, but is not limited to, BLAST (Altschul et al., J. Mol. Biol. 215: 403-410 (1990)), FASTA (Pearson & Lipman, Proc. Natl. Acad. Sci., USA 85: 2444-2448 (1988)), Smith and Waterman method (Smith and Waterman, J. Mol. Biol. 147: 195-197 (1981)), Needleman and Wunsch method (Needleman and Wunsch, J. Mol. Biol. 48: 443-453 (1970)), and the like. BLAST is typically used. Biological search includes, but is not limited to, stringent hybridization, microarray in which genomic DNA is applied on a nylon membrane or glass plate (microarray assay), PCR, in situ hybridization, and the like. Herein, genes used in the present invention are intended to include corresponding genes identified by such electronic or biological search.

An amino acid sequence with one or more amino acid insertions, substitutions, deletions, or additions to one or both ends thereof can be used as a functional equivalent of the invention. As used herein, “amino acid sequence with one or more amino acid insertions, substitutions, deletions, or additions to one or both ends thereof” means that a sequence is modified by a well-known technical method such as site-specific mutagenesis, or by substitution of a plurality of amino acids to the extent that may occur naturally by natural mutations. A modified amino acid sequence of a molecule can be a sequence with, for example, insertion, substitution, deletion, or addition to one or both ends of 1 to 30, preferably 1 to 20, more preferably 1 to 9, still more preferably 1 to 5, and especially preferably 1 to 2 amino acids. A modified amino acid sequence may be an amino acid sequence of a target molecule having one or more (preferably, 1 or several, or 1, 2, 3 or 4) conservative substitutions. Herein, “conservative substitution” refers to substitution of one or more amino acid residues with other chemically similar amino acid residues so that the function of a protein is not substantially modified. Examples thereof include substitution of a certain hydrophobic residue with another hydrophobic residue, substitution of a certain polar residue with another polar residue having the same charge, and the like. A functionally similar amino acid which can be subjected to such substitution is known in the art for every amino acid. Specific examples thereof as a non-polar (hydrophobic) amino acid include alanine, valine, isoleucine, leucine, proline, tryptophan, phenylalanine, methionine, and the like. Examples thereof as a polar (neutral) amino acid include glycine, serine, threonine, tyrosine, glutamine, asparagine, cysteine, and the like. Examples thereof as a (basic) amino acid having a positive charge include arginine, histidine, lysine, and the like. Further, examples thereof as an (acidic) amino acid having a negative charge include aspartic acid, glutamic acid, and the like.

As used herein, a “purified” substance or biological agent (e.g., nucleic acid, protein, or the like) refers to a substance or a biological agent from which at least a part of an agent naturally associated with the biological agent has been removed. Thus, the purity of a biological agent in a purified biological agent is higher than the purity in the normal state of the biological agent (i.e., concentrated). The term “purified” as used herein refers to the presence of preferably at least 75% by weight, more preferably at least 85% by weight, still more preferably at least 95% by weight, and most preferably at least 98% by weight of the same type of a biological agent. A substance used in the present invention is preferably a “purified” substance. As used herein, “isolation” refers to removing at least one of any accompanying substance in a naturally-occurring state. For example, extraction of a specific gene sequence from a genomic sequence can also be referred to as isolation.

As used herein, a “corresponding” amino acid or nucleic acid refers to an amino acid or a nucleotide which has, or is expected to have, in a certain polypeptide molecule or polynucleotide molecule, similar action as a predetermined amino acid or nucleotide in a benchmark polypeptide or a polynucleotide, and for enzyme molecules, refers to an amino acid which is present at a similar position in an active site and makes a similar contribution to catalytic activity. For example, for an antisense molecule, it can be a similar part in an ortholog corresponding to a specific part of the antisense molecule. It is preferable to define identical residues when investigating a corresponding amino acid. A corresponding amino acid can be a specific amino acid subjected to, for example, cysteination, glutathionylation, S—S bond formation, oxidation (e.g., oxidation of methionine side chain), formylation, acetylation, phosphorylation, glycosylation, myristylation, or the like. Alternatively, a corresponding amino acid can be an amino acid responsible for dimerization. Such a “corresponding” amino acid or nucleic acid may be a region or a domain (e.g., V region, D region, or the like) over a certain range. Thus, it is referred herein as a “corresponding” region or domain in such a case.

As used herein, a “marker (substance, protein, or gene (nucleic acid))” refers to a substance which serves as an indicator for tracking whether a subject is in a certain state (e.g., the level or presence of a normal cell state, a transformed state, a disease state, a disorder state, a proliferation ability, or a differentiated state), or whether there is risk thereof. Examples of such a marker include genes (nucleic acid=DNA level), gene products (mRNA, protein, and the like), metabolites, enzymes, and the like. In the present invention, detection, diagnosis, preliminary detection, prediction, or advance diagnosis of a certain state (e.g., a disease such as differentiation disorder) can be materialized using an agent or means specific to a marker associated with the state, or a composition, a kit, a system or the like comprising them. As used herein, “gene product” refers to mRNA or a protein encoded by a gene.

As used herein, “subject” refers to an entity which is to be subjected to diagnosis, detection, or the like in the present invention (e.g., an organism such as a human, an organ or a cell which has been taken out from an organism, or the like).

As used herein, a “sample” refers to any substance obtained from a subject or the like, and includes, for example, a cell or the like. Those skilled in the art can appropriately select a preferable sample based on the descriptions herein.

As used herein, an “agent” is used in a broad sense, and may be any substance or other elements (e.g., energy such as light, radiation, heat, and electricity) as long as the intended object can be attained. Examples of such a substance include, but are not limited to, proteins, polypeptides, oligopeptides, peptides, polynucleotides, oligonucleotides, nucleotides, nucleic acids (e.g., including DNA such as cDNA and genomic DNA, and RNA such as mRNA), polysaccharides, oligosaccharides, fats, organic small molecules (e.g., hormones, ligands, information transmitting substances, organic small molecules, molecules synthesized by combinatorial chemistry, small molecules which can be utilized as a medicine (e.g., a low molecular weight ligand), and the like), and composite molecules thereof. Representative examples of an agent specific to a polynucleotide include, but are not limited to, a polynucleotide having complementarity with certain sequence homology (e.g., 70% or more sequence identity) relative to the sequence of the polynucleotide, a polypeptide such as a transcription factor binding to a promoter region, and the like. Representative examples of an agent specific to a polypeptide include, but are not limited to, an antibody specifically directed to the polypeptide or a derivative or an analog thereof (e.g., single chain antibody), a specific ligand or receptor when the polypeptide is a receptor or a ligand, a substrate when the polypeptide is an enzyme, and the like.

As used herein, a “detection agent” in a broad sense refers to any agent capable of detecting a subject of interest.

As used herein, a “diagnostic agent” in a broad sense refers to any agent with which a state of interest (e.g., a disease or the like) can be diagnosed.

The detection agent of the invention may be a complex or a composite molecule in which another substance (e.g., label or the like) is bound to a portion enabled to be detected (e.g., antibody or the like). As used herein, a “complex” or a “composite molecule” refers to any construct comprising two or more parts. For example, when one of the parts is a polypeptide, the other part may be a polypeptide or another substance (e.g., a sugar, a lipid, a nucleic acid, a different hydrocarbon, or the like). As used herein, two or more parts constituting the complex may be bound by a covalent bond or another bond (e.g., a hydrogen bond, ionic bond, hydrophobic interaction, Van der Waals force, or the like). When the two or more parts are polypeptides, this can also be called a chimeric polypeptide. Thus, as used herein, a “complex” encompasses molecules obtained by connecting a plurality of kinds of molecules such as a polypeptide, a polynucleotide, a lipid, a sugar, and a small molecule.

As used herein, “interaction”, in the context of two substances, refers to a force (e.g., intermolecular force (Van der Waals force), a hydrogen bond, hydrophobic interaction, or the like) being exerted between a substance and the other substance. Generally, the two interacting substances are in an associated or a bound state.

The term “bond” as used herein refers to physical interaction or chemical interaction between two substances or between combinations thereof. The bond includes an ionic bond, a non-ionic bond, a hydrogen bond, a Van der Waals bond, hydrophobic interaction, and the like. Physical interaction (bond) can be direct or indirect, where indirect bond is formed through or due to the effect of another protein or compound. A direct bond refers to interaction, which is not formed through or due to the effect of another protein or compound and involves substantially no other chemical intermediate. The degree of expression of the marker of the invention or the like can be measured by measuring the bond or interaction.

Thus, as used herein, an “agent” (or a detection agent or the like) which “specifically” interacts with (or binds to) a biological agent such as a polynucleotide or a polypeptide includes an agent whose affinity to the biological agent such as a polynucleotide or a polypeptide is typically equal to or higher than, preferably significantly (e.g., statistically significantly) higher than the affinity to other unrelated polynucleotide or polypeptide (particularly those with less than 30% identity). Such affinity can be measured, for example, by a hybridization assay, a binding assay, or the like.

As used herein, a first substance or agent “specifically” interacting with (or binding to) a second substance or agent refers to a first substance or agent interacting with (or binding to) the second substance or agent with higher affinity than that to a substance or agent other than the second substance or agent (particularly another substance or agent that is present in a sample containing the second substance or agent). Examples of interaction (or bond) specific to a substance or an agent include, but are not limited to, a ligand-receptor reaction, hybridization in nucleic acids, an antigen-antibody reaction in proteins, an enzyme-substrate reaction, and when both a nucleic acid and a protein are involved, a reaction between a transcription factor and a binding site of the transcription factor and the like, protein-lipid interaction, nucleic acid-lipid interaction, and the like. Thus, when both of the substances or agents are nucleic acids, a first substance or agent “specifically interacting” with a second substance or agent encompasses the first substance or agent having complementarity to at least a part of the second substance or agent. For example, when both of the substances or agents are proteins, examples of “specific” interaction (or bond) of a first substance or agent with a second substance or agent include, but are not limited to, interaction by an antigen-antibody reaction, interaction by a receptor-ligand reaction, enzyme-substrate interaction, and the like. When two kinds of substances or agents include a protein and a nucleic acid, “specific” interaction (or bond) of a first substance or agent with a second substance or agent encompasses interaction (or bond) between a transcription factor and a binding region of a nucleic acid molecule which is a target of the transcription factor.

As used herein, “detection” or “quantification” of polynucleotide or polypeptide expression can be attained, for example, by using an appropriate method including mRNA measurement and an immunological measuring method, which includes binding or interaction with a marker detection agent. This can be measured in the present invention with the amount of PCR product. Examples of a molecular biological measuring method include a Northern blotting method, a dot blotting method, a PCR method, and the like. Examples of an immunological measuring method include, as a method, an ELISA method using a microtiter plate, an RIA method, a fluorescent antibody method, a luminescence immunoassay (LIA), an immunoprecipitation method (IP), a single radical immuno-diffusion method (SRID), turbidimetric immunoassay (TIA), a Western blotting method, an immunohistological staining method, and the like. Further, examples of a quantification method include an ELISA method, an RIA method, and the like. Detection or quantitation can also be performed by a genetic analysis method using an array (e.g., DNA array or protein array). The DNA array is extensively reviewed in “Saibo Kogaku Bessatsu “DNA maikuroarei to saishin PCR method” [Cell Technology, separate volume, “DNA Microarray and Advanced PCR method], edited by Shujunsha Co., Ltd.). A protein array is described in detail in Nat Genet. 2002 December; 32 Suppl: 526-532. Examples of a method for analyzing gene expression include, but are not limited to, RT-PCR, a RACE method, an SSCP method, an immunoprecipitation method, a two-hybrid system, in vitro translation, and the like in addition to the aforementioned methods. Such additional analysis methods are described, for example, in Genomu Kaiseki Jikkenho/Nakamura Yusuke Labo/Manuaru [Genome Analysis Experimental Method, Nakamura Yusuke Lab. Manual], edited by Yusuke Nakamura, Yodosha Co., Ltd. (2002) and the like. The entire descriptions therein are incorporated herein by reference.

As used herein, “means” refers to anything which can serve as a tool for attaining a certain objective (e.g., detection, diagnosis, or therapy). As used herein, “means for selective recognition (detection)” especially refers to means which can recognize (detect) a certain subject differently from others.

The present invention is useful as an indicator of a state of an immune system. Accordingly, the present invention can be used to identify an indicator of a state of an immune system to find the state of a disease.

As used herein, a “(nucleic acid) primer” refers to a substance required for initiation of a reaction of a polymer compound to be synthesized in a polymer synthesizing enzyme reaction. In a reaction of synthesizing a nucleic acid molecule, a nucleic acid molecule (e.g., DNA, RNA, or the like) complementary to a part of a sequence of a polymer compound to be synthesized can be used. As used herein, a primer can be used as marker detection means.

Examples of a nucleic acid molecule which is generally used as a primer include molecules having a nucleic acid sequence having a length of at least 8 consecutive nucleotides, which is complementary to a nucleic acid sequence of a gene of interest (e.g., marker of the invention). Such a nucleic acid sequence can be a nucleic acid sequence with a length of preferably at least 9 consecutive nucleotides, more preferably at least 10 consecutive nucleotides, still more preferably at least 11 consecutive nucleotides, at least 12 consecutive nucleotides, at least 13 consecutive nucleotides, at least 14 consecutive nucleotides, at least 15 consecutive nucleotides, at least 16 consecutive nucleotides, at least 17 consecutive nucleotides, at least 18 consecutive nucleotides, at least 19 consecutive nucleotides, at least 20 consecutive nucleotides, at least 25 consecutive nucleotides, at least 30 consecutive nucleotides, at least 40 consecutive nucleotides, or at least 50 consecutive nucleotides. A nucleic acid sequence used as a probe includes nucleic acid sequences which are at least 70% homologous, more preferably at least 80% homologous, still more preferably at least 90% homologous, or at least 95% homologous to the aforementioned sequences. A sequence suitable as a primer can vary depending on the nature of a sequence which is intended to be synthesized (amplified), but those skilled in the art can appropriately design a primer depending on the intended sequence. Design of such a primer is well known in the art. Designing may be performed manually or by using a computer program (e.g., LASERGENE, PrimerSelect, or DNAStar).

As used herein, a “probe” refers to a substance that can be means for search, used in a biological experiment such as in vitro and/or in vivo screening. Examples thereof include, but are not limited to, a nucleic acid molecule comprising a specific base sequence or a peptide comprising a specific amino acid sequence, a specific antibody or a fragment thereof, and the like. As used herein, the probe can be used as means for marker detection.

As used herein, “diagnosis” refers to identification of a variety of parameters associated with a disease, disorder, state, or the like in a subject to judge the current or future status of such a disease, a disorder, a state, or the like. By using the method, the apparatus, or the system of the present invention, the state in the body can be examined. A variety of parameters such as a disease, a disorder, or a state in a subject, a formulation or a method for treatment or prevention to be administered can be selected using such information. As used herein, in a narrow sense, “diagnosis” refers to diagnosis of the current status, while encompassing “early diagnosis”, “presumptive diagnosis”, “advance diagnosis”, and the like in a broad sense. Since the diagnosis method of the invention, in principle, can utilize what has come from a body and can be implemented without a healthcare professional such as a doctor, the method is industrially useful. As used herein, “presumptive diagnosis, advance diagnosis, or diagnosis” in particularly may be called “assistance” in order to clarify that the method can be implemented without a healthcare professional such as a doctor.

A procedure of formulating a diagnostic agent or the like of the invention as a drug or the like is known in the art and is described, for example, in Japanese Pharmacopoeia, U.S. Pharmacopoeia, and other countries' Pharmacopoeias. Thus, those skilled in the art can determine the amount to be used from the descriptions herein without undue experiments.

### DESCRIPTION OF PREFERRED EMBODIMENTS

Preferred embodiments of the present invention are described below. Embodiments described below are provided to facilitate the understanding of the present invention. It is understood that the scope of the present invention should not be limited to the following descriptions. Thus, it is apparent that those skilled in the art can make appropriate modifications within the scope of the present invention by referring to the descriptions herein. Those skilled in the art can appropriately combine any embodiments.

<Epitope Clustering Technology>

In one aspect, the present invention provides a method for classifying whether a first immunological entity and a second immunological entity are identical or different for an epitope to be bound thereby, the method comprising the steps of: (1) identifying conserved regions of amino acid sequences of the first immunological entity and the second immunological entity; (2) producing three-dimensional structure models of the first immunological entity and the second immunological entity; (3) superimposing the conserved regions of the first immunological entity and the conserved regions of the second immunological entity in the three-dimensional structure models; (4) determining similarity between non-conserved regions of the first immunological entity and non-conserved regions of the second immunological entity in the three-dimensional structure models after the superimposition; and (5) judging whether an epitope binding to the first immunological entity and an epitope binding to the second immunological entity are identical or different based on the similarity.

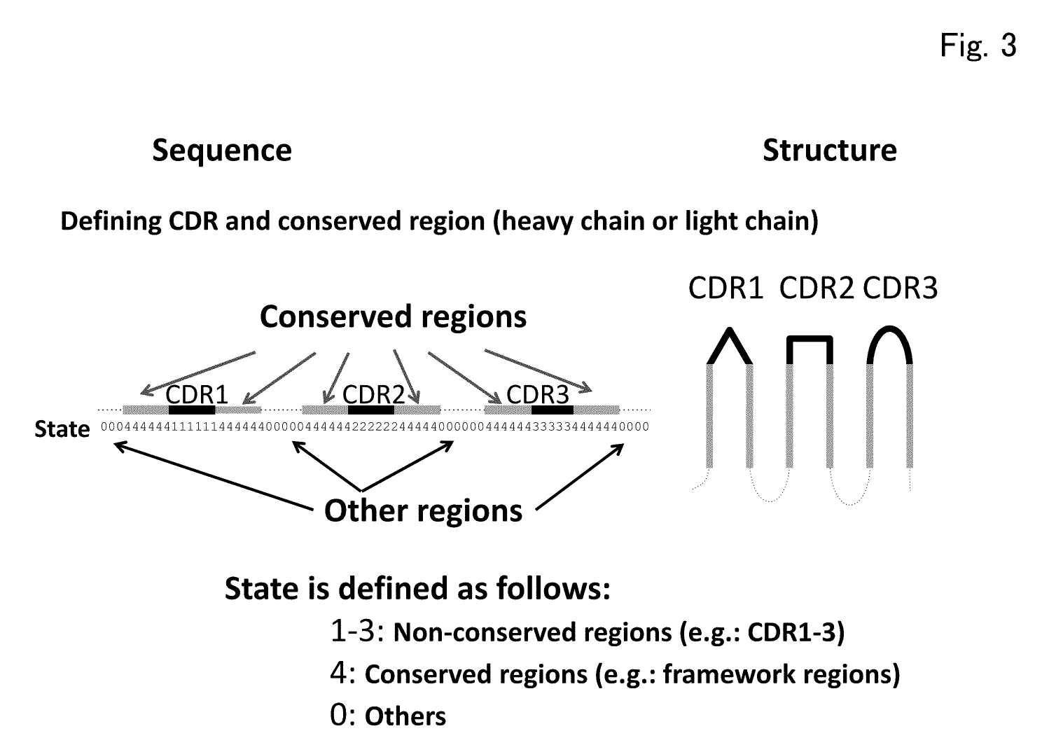

In this regard, the step of identifying conserved regions of amino acid sequences of the first immunological entity and the second immunological entity identifies conserved regions of sequences of immunological entities. Identification can be performed using alignment, three-dimensional structure model, or the like. In one preferred embodiment, a conserved region comprises a framework region or a part thereof, and/or a non-conserved region comprises a complementarity-determining region (CDR) or a part thereof. The conserved region of the first immunological entity has a corresponding relationship to the conserved region of the second immunological entity. In one embodiment, the identification step can decompose a sequence into conserved regions and non-conserved regions. In such a case, the sequence can be divided into framework regions and CDR regions in a preferred embodiment. There are many frameworks, or “numbering” methodologies (Kabat, Chothia, and the like) as methods for describing a CDR region from an amino acid sequence of an immunological entity such as an antibody. They are different in the details, but are qualitatively the same. It is important for an algorithm of the invention to use a common framework independent on the method of separation into CDRs or frameworks, such as assigning an identical number to residues that are three-dimensional structurally identical. This step, formality wise, assigns a region number to each amino acid residue. For practicing the invention, it is not essential to divide into conserved regions and non-conserved regions. The intention of the present invention is to use a structurally universally conserved portion (i.e., conserved region, generally a region called a framework, which may be a portion thereof) for preparation to enable superimposition of structures. One of the important characteristics is to select a region for this purpose. In a representative example illustrated in FIG. 3, 1 to 3 are each CDRs, 4 is a framework region, and 0 is others (FIG. 3).

The step of producing three-dimensional structure models of the first immunological entity and the second immunological entity can make a three-dimensional structure model by a common methodology. In this regard, a preferred embodiment can produce a three-dimensional structure model of the framework region or a part thereof and the CDR or a part thereof for each of the first immunological entity and the second immunological entity. A three-dimensional structure modeling of a variable region of an immunological entity is performed in this manner. As is known in the art, there are many methodologies for three-dimensional structure modeling of a variable region of an immunological entity (homology modeling, molecular dynamics calculation, fragment assembly, combinations thereof, and the like). The details of such three-dimensional structure modeling methodologies are irrelevant to the algorithm of the invention. Any modeling methodology can be applied. However, the accuracy of clustering or grouping is dependent on the accuracy of three-dimensional modeling. In particular, the accuracy of a CDR region, especially CDR-H3, which is the most challenging for structure modeling, is important for accurate grouping based on phenotypes. In other words, it is desirable to use a three-dimensional structure model with as much accuracy as possible from the viewpoint of clustering algorithms. If available, a structure that is experimentally determined can be used.

The step of superimposing the conserved regions (e.g., framework regions or a part thereof) of the first immunological entity and the conserved regions (e.g., framework regions or a part thereof) of the second immunological entity in the three-dimensional structure models materialize superimposition of conserved regions (e.g., framework regions or parts thereof). Framework structures of immunological entities of the same type are sufficiently similar, so that structural superimposition with an error of about 1 angstrom is possible. This is why it is called a framework structure. Various methods (matrix diagonalization and minimalization of root mean square deviation using singular value decomposition are the most prominent) have already been reported for such superimposition. Meanwhile, any algorithm can be used because the algorithm of the invention is not dependent on the specific superimposition methodologies. The structures of all unique antibody pairs can be compared and structures superimposition of conserved regions (e.g., framework region or a part thereof) can be performed, based on the selected superimposition methodology.

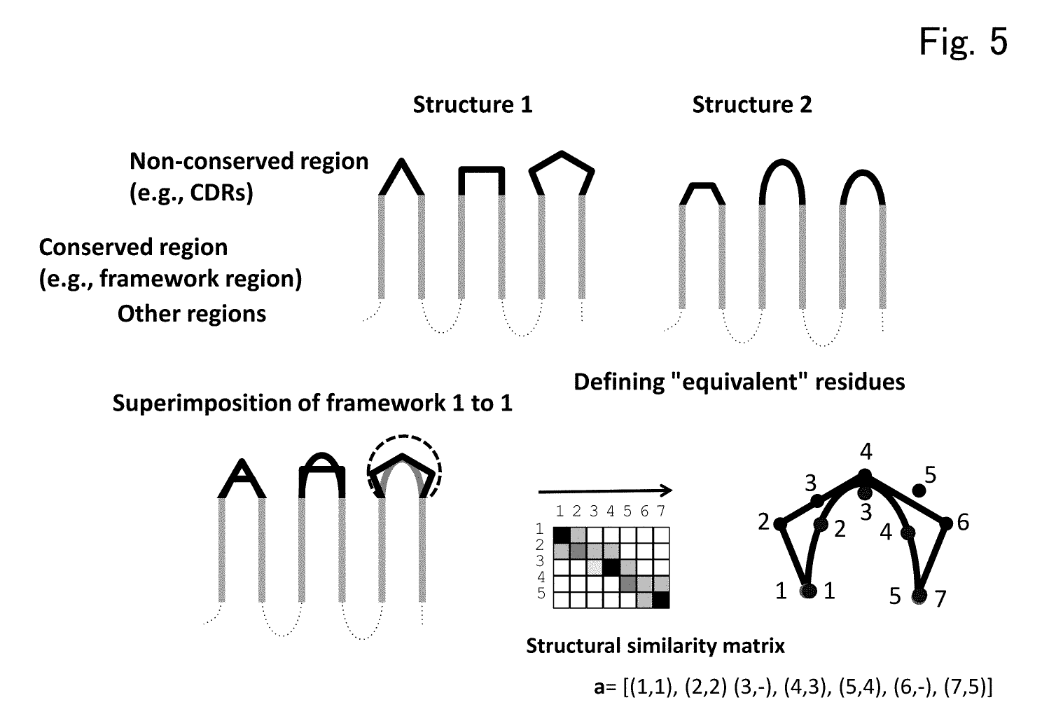

The step of determining similarity between non-conserved regions (e.g., CDR) of the first immunological entity and non-conserved regions (e.g., CDR) of the second immunological entity in the three-dimensional structure models after the superimposition performs similarity calculation (also referred to as structural similarity calculation for similarity calculation of a structure). Identical residues can also be defined as needed. The identical residue can be defined by, for example, calculating similarity (for example, of a CDR region and a framework region) using a model of structurally superimposed immunological entities. Since the difference in lengths of non-conserved regions (e.g., CDR region) between antibodies make it difficult to process, it is desirable to first “align” amino acid residues so that the similarity thereof can be evaluated. A very large number of protein structure alignment methodologies have been discussed in conventional art. A common methodology can be used when two structures are already structurally superimposed by calculating a structural similarity matrix for all amino acid residues of a given non-conserved region (e.g., CDR region) pair (FIG. 5). Further, structures with a high similarity score can be aligned based on dynamic programming. Such a case can use, in addition to the aforementioned example, the Monte Carlo method (e.g., DALI), combination extension method, SSAP method, and the like (Poleksic A (2009). “Algorithms for optimal protein structure alignment”. Bioinformatics. 25 (21): 2751-2756) can be referred to, but the example is not limited thereto) There are other methodologies for expressing similarity. A methodology for giving a positive value for amino acids that spatially overlap and a value near 0 for those with little overlap. The next step is calculation of “alignment” of amino acids using dynamic programming or the like, which deems an amino acid at r₁ as the same as an amino acid at r₂. There are already many alignment methodologies, and any of the methodologies can be used. In this regard, it is preferable to use a methodology belonging to “global alignment” methodologies. This is because the first and last positions of a CDR are approximately identical. The result of alignment can be expressed in a list consisting of all r₁ and r₂ pair information (see FIG. 5).

For similarity calculation, a “feature” for quantifying similarity/dissimilarity is then calculated from two alignments. For example, the following items can be considered.

(a) Difference in lengths. A value is represented as an absolute value (|N₁−N₂|), relative value such as 2*(N₁−N₂)/(N₁+N₂) or (N₁−N₂)/Nₐ, standardized value, or the like, wherein Nₐ is the length of the alignment. Alternatively, the value can be a difference in the lengths of a loop (can be ΔLoop, maximum difference in CDR loop lengths, or the like).
(b) Sequence similarity. Generally, an amino acid mutation is calculated by an amino acid substitution matrix (e.g., BLOSUM62), and a penalty is given when an alignment has a gap). Further, the number of identical amino acids is simply counted in some cases.
(c) Structural similarity. Any methodology that can evaluate a three-dimensional structure can be employed. One of the features of the present invention is in evaluating the structural similarity of a three-dimensional structure, whereby an epitope clustering technology with high accuracy is attained. Examples of preferred methodologies include the use of technology that can normalize to a value from 0 to 1.

The above is merely one example. A more complex function type comprising more terms can be used to perform the present invention.

The step of judging whether an epitope binding to the first immunological entity and an epitope binding to the second immunological entity are identical or different based on the similarity performs structural similarity calculation of non-conserved regions (e.g., variable region such as a CDR) of two immunological entities (e.g., antibody). Similarity or dissimilarity of two antibodies can be quantified by various methods by using a set of features for describing similarity of various features represented by non-conserved region (CDR or the like), conserved region (framework or the like), and the like. A representative non-limiting example of the methodology is a regressive scheme, such as a sum of weighted similarity/dissimilarity features. In a preferred embodiment, a more refined method can input these features into various neural network methods or a machine learning algorithm such as support vector machine or random forest.

In a special case where an immunological entity binder (e.g., antigen) is already known or some of the antibody targets are known, the step of evaluating similarity of the invention can include these known cases to clustering as an application. In other words, an immunological entity binder (e.g., antigen) of an immunological entity (e.g., antibody)/epitope can be predicted by using an immunological entity (e.g., antibody) with a known epitope/immunological entity binder (e.g., antigen).

Epitopes classified into a cluster described herein can be associated with biological information. For example, a carrier of the antibody can be associated with a known disease, disorder, or biological condition based on one or more clusters of epitope identified based on the classification method of the invention.

Examples of diseases, disorders, or biological conditions that can be involved in the present invention include infections by a foreign object (e.g., bacteria, virus, or the like), as well as self entities recognized as non-self (e.g., neoplasm (cancer or tumor) and entities associated with autoimmune diseases). An immune system functions to distinguish molecules endogenous to an organism (“self” molecule) from substances exogenous or foreign to the organism (“non-self molecule”). The immune system has two types of adaptive responses (humoral response and cell-mediated response) to a foreign object based on the constituent component mediating the response. A humoral response is mediated by an antibody, while cell-mediated response involves cells classified as lymphocytes. In recent anticancer and antiviral strategies, use of the host immune system as means of anticancer or antiviral treatment of therapy is an important strategy. The classification or clustering technologies of the invention can also be applied in both humoral response and cell-mediated response strategies.

The immune system functions through three stages (recognition, activation, and effector) in defending the host from a foreign object. In the recognition stage, the immune system recognizes the presence of an exogenous antigen or an intruder and notifies its presence. An exogenous antigen can be, for example, a foreign object (cell surface marker from a viral protein or the like), or a cell surface marker of a cell (cancer cell) that can be recognized as non-self, or the like. When the immune system recognizes an intruder, antigen-specific cells of the immune system proliferate and differentiate in response to an intruder-induced signal (activation stage). The final stage is the effector stage for the effector cells of the immune system to neutralize the detected intruder in response thereto. Effector cells play the role of carrying out an immune response. Examples of effector cells include B cells, T cells, natural killer (NK) cells, and the like. B cells produce an antibody against an intruder, and the antibody, in combination with a complement system, guides the cell or organism comprising a specific target epitope (immunological entity binder such as an antigen) to its destruction. T cells are categorized into types such as helper T cells, regulatory T cells, and cytotoxic T cells (CTL cells). Helper T cells secrete a cytokine and stimulate the growth of other cells or the like to enhance the efficacy of an immune response. Regulatory T cells downregulate an immune response. CTL cells directly dissolves/melts and destroys cells presenting an exogenous antigen on the surface. NK cells are understood to recognize and destroy virally infected cells, malignant tumor cells, or the like. Therefore, classification of epitopes targeted by these effector cells and linking the epitopes to a disease, disorder, or biological condition plays a very important role in the efficacy of therapy or diagnosis.

In this manner, T cells are antigen specific immune cells that function in response to a specific antigen signal. B lymphocytes and antibodies produced thereby are also antigen specific objects. The present invention enables these specific immunological entity binders (e.g., antigen) to be classified and clustered using an epitope cluster by the final function (association with a specific disease, disorder, or biological condition).

As discussed above, B cells respond to free or soluble antigens, but T cells do not. For the T cells to response to an antigen, the antigens need to be processed by a peptide and linked to a presentation structure encoded by a major histocompatibility complex (MHC) (called “MHC restriction”). T cells distinguish self cells from non-self cells by this mechanism. If an antigen is not presented by a recognizable MHC molecule, T cells d₀ not recognize an antigen signal. T cells specific to a peptide bound to a recognizable MHC molecule bind to an MHC peptide complex, and an immune response progresses. MHC has two classes (MHC class I and MHC class II). It is understood that CD4⁺ T cells preferentially interact with MHC class II proteins, while cytotoxic T cells (CD8⁺) preferentially interact with MHC class I. These MHC proteins of both classes are transmembrane proteins comprising the majority of the structure thereof on the external surface of a cell, having a peptide bond space on the outside thereof. Fragments of both endogenous and exogenous proteins are bound and presented to the extracellular environment in this space. At this time, cells known as professional antigen presenting cells (pAPCs) present an antigen to T cells using an MHC protein, and induce a pathway for differentiation and activation of T cells using various specific costimulatory molecules to materialize the effect of the immune system. The classification and clustering technologies for epitopes of the invention provide an applied method that could not be provided with conventional art for the therapy or diagnosis involving such MHCs.

For non-self entities, an applied method for therapy or diagnosis can be provided by sufficiently utilizing conventional immune system, but further creativity could be required for self entities. This is because cancer cells and the like have the same origin as normal cells and are substantially identical to normal cells at gene levels. However, cancer cells are known to present tumor associated antigens (TuAA). In addition, the immune system of a subject can be utilized to attack cancer cells by utilizing the antigen or another immunological entity binder. Such tumor associated antigens can also classify and cluster epitopes to an indicator with the technology of the invention. For example, a tumor associated antigen can be applied to anticancer vaccine or the like. For example, a conventional technology using the entire activated tumor cell is disclosed in U.S. Pat. No. 5,993,828. Alternatively, a technology applying a composition comprising an isolated tumor antigen has been attempted (e.g., Krishnadas D K et al., Cancer Immunol Immunother. 2015 October; 64(10): 1251-60). A genetically modified T cell (also called CAR-T) using a chimeric antigen receptor (CAR) that recognizes an identified epitope can also be used. An immunotherapy using an immune checkpoint inhibitor or the like based on the action related to an immune checkpoint such as PD-1 or PD-L1 has also drawn attention recently. PD-1 binds to a PD-1 ligand (PD-L1 and PD-L2) expressed in an antigen presenting cell and transmits a suppressive signal to lymphocytes to downregulate the activation state of lymphocytes. PD-1 ligands are expressed in various human tumor tissues other than antigen presenting cells. It is understood that there is a negative correlation between the expression of PD-L1 in resected tumor tissue and post-op survival period in malignant melanoma. It is understood that the cytotoxic activity recovers if binding of PD-1 and PD-L1 in PD-1 antibodies or PD-L1 antibodies is inhibited. A sustained antitumor effect can be exhibited by activation of antigen specific T cells and enhancement of cytotoxic activity to cancer cells (e.g., nivolumab or the like). The epitope classification or clustering method of the invention can also be applied to the mechanism of restoring the downregulation mechanism of immune activity.

For vaccines, the epitope classification or clustering method of the invention can be applied to viral diseases. As a vaccine for a virus, attenuated vaccine, inactivated vaccine, subunit vaccine, and the like are utilized. While the success rate of subunit vaccines is not high, successful examples in a recombinant hepatitis B vaccine based on an envelope protein and the like have been reported. Since a biological condition can be suitably associated using the epitope classification or clustering method of the invention, it is understood that efficacy with a subunit vaccine or the like is also improved. It is also understood that suitable quantitative evaluation of clusters leads to evaluation of efficacy of vaccines. Stratification is also possible by comparison with cases where a vaccine is effective. It is understood that the efficacy is improved or the possibility of distribution in the market is improved as a result. In fact, a result of identifying a cluster reacting to a vaccine in silico using the methodology of the invention has been shown.

In one embodiment, examples of immunological entities that can be used in the epitope classification or clustering method of the invention include an antibody, an antigen binding fragment of an antibody, a B cell receptor, a fragment of a B cell receptor, a T cell receptor, a fragment of a T cell receptor, a chimeric antigen receptor (CAR), a cell comprising one or more of them (e.g., T cell comprising a chimeric antigen receptor (CAR) (CAR-T)) and the like.

In one specific embodiment, the decomposing step that can be used in the present invention can use any methodology as long as an antibody sequence can be divided into framework regions and CDR regions. Further, any method can be used for describing a CDR region from an antibody amino acid sequence. There are many frameworks for such methods. The method can be performed based on various numbering methodologies such as, but not limited to, Kabat, Chotia, modified Chotia, IMGT, and Honnegger. It is understood that the method of the invention is not dependent on the technology used, but rather any technology capable of the same classification can be used. The details thereof are different, but the technologies are qualitatively the same. It is important for the algorithm of the inventors to use a common framework. As a format, the step assigns a region number to each amino acid residue. In the exemplary scheme depicted in FIG. 3, 1 to 3 are each CDRs, 4 is a framework region, and 0 are others. While the present invention is not limited to the following, it can be advantageous to use the following methodology: using a numbering methodology for assigning the same number to residues that are considered structurally equivalent; and selecting and defining a structurally stable residue in many antibodies as a framework. Available structural information is increasing daily, such that the definitions are preferably updated as appropriate.

In one specific embodiment, production (modeling) of a three-dimensional structure model that can be utilized in the present invention can use any methodology, as long as it is capable of three-dimensional structure modeling of an antibody variable region. Such modeling is performed based on a modeling methodology such as homology modeling, molecular dynamics calculation, fragment assembly, Monte Carlo simulation, optimization methodology such as simulated annealing, or a combination thereof. It is understood that the method of the invention is not dependent on the modeling methodology used, but rather the same modeling is possible with any modeling technology. The algorithm of the inventors is not dependent on the details of such three-dimensional structure modeling methodologies. However, the accuracy of clustering or grouping is dependent on the accuracy of a three-dimensional modeling. In particular, the accuracy of a CDR region, especially CDR-H3, which is the most challenging for structure modeling, is important for accurate grouping based on phenotypes. Thus, enhancement of the accuracy thereof is preferred. In other words, it is desirable to use a three-dimensional structure model with as much accuracy as possible from the viewpoint of clustering algorithms. If available, a structure that is experimentally determined can be used. In one advantageous embodiment for modeling, precise modeling of a CDR heavy chain 3 enables classification with higher accuracy, but the present invention is not limited thereto. A methodology capable of attaining highly precise modeling can be advantageous, but the present invention is not limited thereto.

In another embodiment, structure prediction can perform sequence alignment as the first step in structure prediction and then perform three-dimensional structure modeling. For example, a query sequence (can be denoted as q) for which a structure prediction is desirable can be efficiently aligned with respect to multiple sequence alignments (MSA, can be denoted as m) without changing the alignment between templates (Katoh, K. and Standley, D. M. MAFFT multiple sequence alignment software version 7: improvements in performance and usability. Mol Biol Evol 2013; 30(4): 772-780.) In a specific embodiment, the length of a non-conserved region such as CDR can be first estimated with alignment to framework MSA, and templates that naturally formed a pair with the highest overall framework score (e.g., BCR_L-H or TCR_A-B) can be selected to define the directionality of two framework templates. Next, a full length query sequence can be aligned to suitable MSA for non-conserved regions such as each CDR. Although not wishing to be bound by any theory, a full length sequence can be used in CDR MSA or the like because a residue outside of CDRs can contribute to the stability thereof. For example, a CDR template with the highest score can be transplanted into the framework template with the highest score by using RMSD superimposition of four residues in front and back of CDRs as an anchor. In each step, a mismatch is monitored. If a mismatch exceeds a threshold value, the template with the highest score can be replaced with a non-optimal template. Side chains that are different between query and template can be reconstructed using a conformation observed frequently in a corresponding MSA row.

In a specific embodiment, a superimposing step that can be utilized in the present invention may use any methodology, as long as framework regions can be superimposed. The antibody framework structures of the same species are sufficiently similar, so that structures can be superimposed with an error of about 1 angstrom or several angstroms (e.g., 2 Å, 3 Å, 4 Å, 5 Å, 6 Å, 7 Å, 8 Å, 9 Å, 10 Å, or the like). Such superimposition can be performed based on various superimposition methods such as, but not limited to, known least squares method, matrix diagonalization, minimalization of root mean square deviation using singular value decomposition, or optimization of structural similarity score based on dynamic programming. It is understood that the method of the invention is not dependent on the superimposition method used, but rather any superimposition technology can perform the same superimposition. The algorithm of the inventors is not dependent on these specific superimposition methodologies. The structures of all unique antibody pairs can be compared to superimpose the structures of framework regions based on the selected superimposition methodology. While the present invention is not limited to the following, it can be advantageous to use the following superimposition methodology. Residues that are universally structurally stable across many immunological entities (e.g., antibodies) are selected as a framework region and superimposed, whereby similarity of structurally variable regions can be more accurately evaluated.

In a preferred embodiment, it can be advantageous to perform the superimposition in the present invention with an error of 1 angstrom or several angstroms (e.g., 2 Å, 3 Å, 4 Å, 5 Å, 6 Å, 7 Å, 8 Å, 9 Å, 10 Å, or the like) or less. This is because the accuracy of classification or clustering can be enhanced.

In a preferred embodiment, identical residues are defined when determining the structural similarity in the present invention. The defining of identical residues that can be performed in the present invention can employ anything, as long as it enables calculation of similarity using a structurally superimposed antibody model (e.g., CDR region and framework region). A CDR region generally having different lengths for each antibody makes it difficult to process. In this regard, in one embodiment, it is advantageous to first “align” amino acid residues to enable evaluation of similarity thereof, but the approach is not limited thereto. Many protein structure alignment methodologies have been discussed. While the general methodology is not limited, examples thereof include calculation of a structural similarity matrix of all amino acid residues of a given CDR pair. This is a methodology that can be used when two structures are already structurally superimposed (FIG. 5).

It is also possible to align those with a high similarity score based on dynamic programming. In a specific embodiment, identical residues that can be used are defined based on alignment. Specific procedures of exemplary alignment that is utilized include the following: 1) calculating a structural similarity matrix of all amino acid residues of a given CDR pair; and 2) aligning based on dynamic programming. If coordinates of two CDRs of the CDR pair are represented by r₁ and r₂, similarity Sₖₗ of any two residues k and l is defined by

$$\begin{matrix}[ Numeral 4 ] & \\ {S}_{kl} = {e}^{- {( \frac{{r}_{1} [ k ] - {r}_{2} [ l ]}{{d}_{0}} )}^{2}} , & ( 1 )\end{matrix}$$

wherein coordinates of k and l are represented as r₁ and r₂, respectively, and r₁[i]−r₂[j] is a vector consisting of a difference between coordinates of two amino acids, and d₀ is an empirically determined parameter. In this regard, a Cα atom or a center-of-mass coordinate is preferably used as the representative coordinate, but the present invention is not limited thereto.

In a preferred embodiment, the methodology for expressing similarity, in determining structural similarity in the present invention, comprises:

(1) calculating a value of

$$[ Numeral 5 ]$$

$${S}_{kl}^{′} = \frac{a}{b + {( {r}_{1} [ k ] - {r}_{2} [ l ] )}^{2}} ,$$

wherein a large value indicates a large overlap; and/or;
(2) calculating alignment of amino acids using a global sequence alignment methodology.

The main concept of this step is to give a positive value to spatially overlapping amino acids (low |r₁[i]−r₂[j]|) and a value close to zero for those with little overlap (high |r₁[i]−r₂[j]|). The next step calculates the alignment of amino acid sequences using dynamic programming or the like. This means that the amino acid at r₁ is considered equivalent to the amino acid at r₂. There are already many sequence alignment methodologies. It is preferable to use a methodology belonging to “global alignment methodologies”. This is because the first and last positions of a CDR are approximately identical, but the present invention is not limited thereto. The result of alignment is a list consisting of information for all r₁ and r₂ pairs exemplified as follows.

r₁[1]r₂[1]

r₁[2]r₂[2]

r₁[3]-

r₁[4]r₂[3] [Numeral 6]

wherein “-” on line 3 in the above example means that an amino acid forming a pair with r₁[3] could not be found in r₂. In the above case, alignment can be described as follows: a=[(1, 1), (2, 2), (3, -), (4, 3) . . . ] (see FIG. 5).

In one embodiment, the structural similarity that can be employed in computation of structural similarity, which can be performed in the present invention, is determined based on at least one of a difference in lengths, sequence similarity, and three-dimensional structure similarity. This is to calculate similarity/“feature” for quantifying similarity from two alignments.

In this regard, the difference in lengths can be expressed as an absolute value (|N₁−N₂|), relative value such as 2*(N₁−N₂)/(N₁+N₂) or (N₁−N₂)/Nₐ, standardized or normalized value, or the like, wherein Na indicates the length of the alignment. Alternatively, this can be defined as the maximum difference in CDR length for all 6 CDRs. This formula is based on the knowledge that dividing by length or averaging CDRs can be considered as having hardly any effect because different epitopes targeted by BCR often differ in terms of the length of CDR in a CDR.

Generally, sequence similarity can be computed by calculating a mutation of an amino acid. Sequence similarity can also be an absolute value or relative value, and may be standardized or normalized. Amino acid mutations are generally calculated with an amino acid substitution matrix (e.g., BLOSUM62). A penalty can be given when an alignment has a gap. Alternatively, the number of identical amino acids can be simply counted. Specific examples of calculating sequence similarity include the following. Specifically, for CDRs, sequence similarity can be specified from the viewpoint of components of BLOSUM62 matrix of aligned residues. If an aligned residue pair consists of amino acids a₁ and a₂ for two immunological entities, and the component in the BLOSUM62 a₁-a₂ matrix is indicated as Bᵢ, while components of elements a₁-a₁ and a₂-a₂ on the diagonal line are indicated as Cᵢ and Dᵢ, the score of a give CDR can be defined as follows.

$$[ Numeral 6 A ]$$

$$SeqSim = {\sum }_{i}^{N} \frac{{B}_{i}}{MAX ( {C}_{i} , {D}_{i} )}$$

The structural similarity can be computed by calculating the similarity using any parameter specifying a structure. The structural similarity can also be an absolute value or relative value, and may be standardized or normalized. If identical residues have been defined, the structural similarity can be calculated, for example, by the following formula as a simple extension thereof:

$$[ Numeral 7 ]$$

$${S}_{12} = {\sum }_{k}^{{N}_{a}} {e}^{- {( \frac{{r}_{1} [ a ( k , 0 ] ] - {r}_{2} [ a [ k , 1 ] ]}{{d}_{0}} )}^{2}} ,$$

wherein Nₐ is the length of alignment, w₁ and w₂ are empirically determined parameters. The advantage of using such a function type is that a value can be normalized to a value from 0 to 1.

Alternatively, structural similarity can be evaluated by further dividing the above formula by N (see Example 3). Previously disclosed theory related to protein structure alignment can be referenced for structural similarity in CDRs or the like (Standley, D. M., Toh, H. and Nakamura, H. Detecting local structural similarity in proteins by maximizing number of equivalent residues. Proteins 2004; 57(2): 381-391.) In a specific embodiment, structural similarity can be computed as an average of 6 CDRs for a subject, but the present invention is not limited thereto.

Computation of structural similarity that can be performed in the present invention can obviously use a more complex function type comprising more terms.

In a preferred embodiment, structural similarity comprises at least three-dimensional structural similarity. This is because the accuracy of epitope classification and clustering can be further improved for more accurate linking by biological significance by calculation using three-dimensional structural similarity.

In one embodiment, the calculation of structural similarity of the invention can use any calculation, as long as structural similarity of two antibody variable regions can be calculated. For example, a regressive scheme, neural network method, or machine learning algorithms such as support vector machine and random forest can be used. In a preferred embodiment, similarity or dissimilarity of two antibodies can be quantified by various methods by using a set of features for describing similarity of CDRs and frameworks. An exemplary methodology is a regressive scheme, such as a sum of weighted similarity/dissimilarity features. As another exemplary embodiment, a more refined method that inputs these features into various neural network methods or a machine learning algorithm such as support vector machine or random forest can be used. A case where support vector machines are used is described below as an example, but those skilled in the art understand that the same result is obtained using other methodologies. The present invention is not dependent on the specific similarity score or details. The key in one embodiment is in applying machine learning or other score functions to describe an antibody pair. In a general embodiment, an immunological entity binder such as an antigen or epitope is not assumed to be known, but in such a case, it is therefore important to predict the degree of match between the antibody pair rather than predicting an antigen or epitope. One of the features is in that classification and clustering of the invention can also be materialized in such a case.

In this regard, the present invention provides a method for generating a cluster of epitopes classified based on the methodology of the invention, wherein the method comprises the step of classifying immunological entities binding to an identical epitope to an identical cluster. In one embodiment, the immunological entities are evaluated by at least one endpoint selected from the group consisting of a property and similarity with a known immunological entity thereof to perform the cluster classification targeting an immunological entity meeting a predetermined baseline. A three-dimensional structure of the epitope can at least partially or fully overlap when a plurality of the epitopes are identical, and an amino acid sequence of the epitope can at least partially or fully overlap when a plurality of the epitopes are identical.

In one embodiment, a specific threshold value can be set for evaluation. For example, structural similarity, sequence similarity, difference in lengths, or the like can have a minimum value of 0 and maximum value of 1, where the threshold value can be set to a value of, for example, 0.8 or greater, 0.85 or greater, 0.9 or greater, 0.95 or greater, 0.99 or greater, or the like, or any value therebetween (e.g., in 0.1 increments).

For example, structural similarity (e.g., StrucSim score) between all immunological entities (antibodies, TCRs, BCRs, or the like) and all immunological entities (antibodies, TCRs, or BCRs) can be calculated. For StrucSim score, a value can be set between 0 and 1. A threshold value can be appropriately determined. For example, about 0.9 can be used to distinguish whether an entity belongs to an identical epitope group or another group. To increase the degree of separation, the threshold value can be appropriately raised. When, for example, about 0.9 is used, the threshold value can be set higher to about 0.95 or the like. A single line can be drawn between portions of a pair with a matching characteristic within the threshold value to make the cluster visible. In doing so, a software such as Python Network X graphviz package can be used.

In a special case where an immunological entity binder (e.g., antigen) is known or a case where some of the antibody target is known when calculating structure similarity of variable regions of two immunological entities (e.g., antibodies), these known cases can be included in clustering as an application. In such a case, an antigen/epitope of an immunological entity (e.g., antibody) can be predicted using the antibody with a known immunological entity binder (e.g., antigen)/epitope. Several methods of use are conceivable as these methodologies, which are described below.

1. When extracting only a similar antibody (or another immunological entity) using similarity to the known antibody of interest (or another immunological entity).
2. When evaluating similarity between representative or all antibodies of each cluster (or another immunological entity) and a known antibody (or another immunological entity) after full or partial clustering.
3. When a single antibody (or another immunological entity) is evaluated to be similar to a plurality of known antibodies (or other immunological entities), the antibody with the highest similarity should be selected. When a plurality of antibodies (or other immunological entities) are evaluated to be similar to a plurality of known antibodies (or other immunological entities) in a single cluster, it is desirable to select a known antibody (or another immunological entity) most suitable in terms of similarity or the number of antibodies (or another immunological entity) determined to be similar, or reevaluate the threshold value for clustering to divide the cluster into a plurality of clusters.
4. There can be one or more known antibodies (or other immunological entities) of interest depending on the objective. When an antigen (or another immunological entity binder) is unknown, 1000 to several 10s of thousands of known antibodies (or other immunological entities) can be used for the purpose of antigen screening.

The above examples typically provide an explanation using an antibody as an example, but it is understood that the same applies to immunological entities other than antibodies.

<Epitope Cluster and Antigens>

In yet another aspect, the present invention provides an epitope or an antigen (or a corresponding immunological entity binder) having a structure identified by the method of the invention or a cluster thereof. The epitopes and the like defined here can have any characteristic described in <Epitope clustering technology” herein, or can be an epitope identified, classified, or clustered by such technologies. In this regard, a method for generating a cluster can include the step of classifying immunological entities binding to an identical epitope to an identical cluster. In a preferred embodiment, the immunological entities can be evaluated by at least one endpoint selected from the group consisting of a property and similarity with a known immunological entity thereof to perform the cluster classification targeting an immunological entity meeting a predetermined baseline. As the baseline that can be employed therein can be, for example, a three-dimensional structure of the epitope can at least partially overlap when a plurality of the epitopes are identical, or an amino acid sequence of the epitope can at least partially overlap when a plurality of the epitopes are identical.

One embodiment of the present invention relates to a classified epitope, a clustered epitope, and an immunological entity binder (e.g., antigen) or polypeptide comprising the epitope.

In this regard, examples of the method for describing (identifying) a classified epitope or clustered epitope include the following. Specifically, a cluster of immunological entities (e.g., antibodies) identified by the methodology of the invention is understood as recognizing an identical epitope at a high accuracy, so that an epitope recognized by the cluster can be identified by similarity evaluation of an immunological entity binder (e.g., antigen) to a known immunological entity (e.g., antibody with a known antigen), experimental antigen screening (or screening of another immunological entity binder), more desirably a mutation experiment of an antigen-antibody pair (or another immunological entity-immunological entity binder), NMR chemical shift, crystal structure analysis, identification of an epitope associated with interaction, or functional evaluation by an in vitro or in vivo experiment. Therefore, even if a known epitope or immunological entity binder (e.g., antigen) and an immunological entity based thereon are provided, epitopes clustered or classified as in the present invention have specific information, can be used in a specific application, and can be considered as having a specific effect and function. In this regard, a new characteristic that is absent in conventional epitopes or immunological entity binders (e.g., antigens) and immunological entities based thereof is provided, such that technical matter with a novel and significant characteristic is provided.

<Program, Medium, and System Configuration>

In one aspect, the present invention provides a program for executing the method of invention. Any characteristic that can be employed herein can be any of the characteristics described in <Epitope clustering technology> herein or a combination thereof. The program of the invention can be a computer program for making a computer execute a method for classifying whether a first immunological entity and a second immunological entity are identical or different for an epitope to be bound thereby, the method comprising the steps of: (A) identifying conserved regions of amino acid sequences of the first immunological entity and the second immunological entity; (B) producing three-dimensional structure models of the first immunological entity and the second immunological entity; (C) superimposing the conserved regions of the first immunological entity and the conserved regions of the second immunological entity in the three-dimensional structure models; (D) determining similarity between non-conserved regions of the first immunological entity and non-conserved regions of the second immunological entity in the three-dimensional structure models after the superimposition; and (E) judging whether an epitope binding to the first immunological entity and an epitope binding to the second immunological entity are identical or different based on the similarity.

In another aspect, the present invention provides a recording medium storing a program for executing the method of the invention. In one embodiment, the recording medium can be a ROM, HDD, or magnetic disk that can be stored internally, or an external storage apparatus such as flash memory such as a USB memory. Any of the characteristics that can be employed therein can be any of the characteristics described in <Epitope clustering technology> herein or a combination thereof. The recording medium of the invention can be a recording medium storing a computer program for making a computer execute a method for classifying whether a first immunological entity and a second immunological entity are identical or different for an epitope to be bound thereby, the method comprising the steps of: (A) identifying conserved regions of amino acid sequences of the first immunological entity and the second immunological entity; (B) producing three-dimensional structure models of the first immunological entity and the second immunological entity; (C) superimposing the conserved regions of the first immunological entity and the conserved regions of the second immunological entity in the three-dimensional structure models; (D) determining similarity between non-conserved regions of the first immunological entity and non-conserved regions of the second immunological entity in the three-dimensional structure models after the superimposition; and (E) judging whether an epitope binding to the first immunological entity and an epitope binding to the second immunological entity are identical or different based on the similarity.

In another aspect, the present invention provides a system comprising a program for executing the method of the invention. Any of the characteristics that can be employed therein can be any of the characteristics described in <Epitope clustering technology> herein or a combination thereof. The system of the invention can be a system for classifying whether a first immunological entity and a second immunological entity are identical or different for an epitope to be bound thereby, the system comprising: (A) a conserved region identifying unit for identifying conserved regions of amino acid sequences of the first immunological entity and the second immunological entity; (B) a three-dimensional structure model producing unit for producing three-dimensional structure models of the first immunological entity and the second immunological entity; (C) a superimposing unit for superimposing the conserved regions of the first immunological entity and the conserved regions of the second immunological entity in the three-dimensional structure models; (D) a similarity determining unit for determining similarity between non-conserved regions of the first immunological entity and non-conserved regions of the second immunological entity in the three-dimensional structure models after the superimposition; and (E) an identity judging unit for judging whether an epitope binding to the first immunological entity and an epitope binding to the second immunological entity are identical or different based on the similarity. The conserved region identifying unit, three-dimensional structure model producing unit, superimposing unit, similarity determining unit, and identity judging unit can be materialized by separate constituent elements, or two or more can be materialized with a single constituent element.

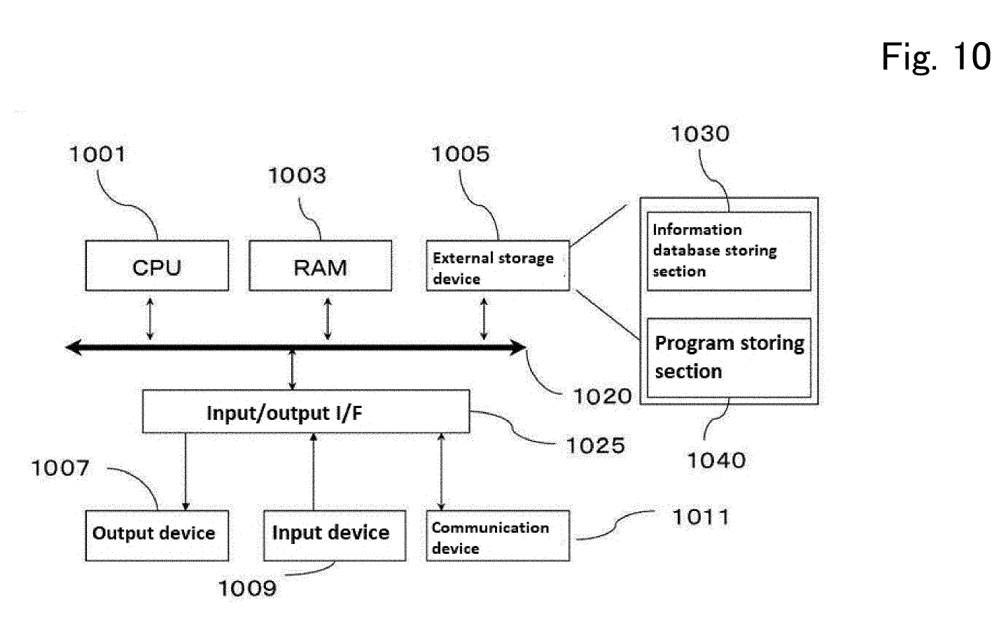

The configuration of system 1 of the invention is now described with reference to the function block diagram in FIG. 10. While the figure depicts a case where the invention is materialized with a single system, it is understood that cases where the invention is materialized with a plurality of systems are encompassed in the scope of the invention.

The system 1000 of the invention is constituted by connecting a RAM 1003, a ROM or HDD or a magnetic disk, an external storage device 1005 such as flash memory such as a USB memory, and an input/output interface (I/F) 1025 to a CPU 1001 built into a computer system via a system bus 1020. An input device 1009 such as a keyboard or a mouse, an output device 1007 such as a display, and a communication device 1011 such as a modem are each connected to the input/output I/F 1025. The external storage device 1005 comprises an information database storing section 1030 and a program storing section 1040. Both are a certain storage area secured within the external storage apparatus 1005.

In such a hardware configuration, various instructions (commands) are inputted via the input device 1009 or commands are received via the communication I/F, communication device 1011, or the like to call up, deploy, and execute a software program installed on the storage device 1005 on the RAM 1003 by the CPU 1001 to accomplish the function of the invention in cooperation with an OS (operating system). Of course, the present invention can be implemented with a mechanism other than such cooperating setup.

In the implementation of the present invention, the amino acid sequences or information equivalent thereof (e.g., nucleic acid sequences encoding the same or the like) of a first immunological entity and a second immunological entity (which can be antibodies, B cell receptors, T cell receptors, or the like) can be inputted via the input device 1009, inputted via the communication I/F, communication device 1011, or the like, or stored in the database storing section 1030. The step of decomposing the amino acid sequences of a first immunological entity and a second immunological entity into framework regions and complementarity-determining regions (CDR) can be executed with a program stored in the program storing section 1040, or a software program installed in the external storage device 1005 by inputting various instructions (commands) via the input device 1009 or by receiving commands via the communication I/F, communication device 1011, or the like. Divided data can be outputted through the output device 1007 or stored in the external storage device 1005 such as the information database storing section 1030. The step of producing three-dimensional structure models of a framework region and a CDR for each of the first immunological entity and second immunological entity can also be executed with a program stored in the program storing section 1040, or a software program installed in the external storage device 1005 by inputting various instructions (commands) via the input device 1009 or by receiving commands via the communication I/F, communication device 1011, or the like. The data of the produced three-dimensional model can be outputted through the output device 1007 or stored in the external storage device 1005 such as the information database storing section 1030. The step of superimposing framework regions of a first immunological entity and the framework regions of a second immunological entity can also be executed with a program stored in the program storing section 1040, or a software program installed in the external storage device 1005 by inputting various instructions (commands) via the input device 1009 or by receiving commands via the communication I/F, communication device 1011, or the like. The generated superimposition data can be outputted through the output device 1007 or stored in the external storage device 1005 such as the information database storing section 1030. The step of determining structural similarity between the CDR of the first immunological entity and the CDR of the second immunological entity in the three-dimensional structure models after the superimposition can also be executed with a program stored in the program storing section 1040, or a software program installed in the external storage device 1005 by inputting various instructions (commands) via the input device 1009 or by receiving commands via the communication I/F, communication device 1011, or the like. The produced structural similarity data can be outputted through the output device 1007 or stored in the external storage device 1005 such as the information database storing section 1030. Defining of identical residues performed for structural similarity can also be executed with a program stored in the program storing section 1040, or a software program installed in the external storage device 1005 by inputting various instructions (commands) via the input device 1009 or by receiving commands via the communication I/F, communication device 1011, or the like. The produced definition of identical residues can be outputted through the output device 1007 or stored in the external storage device 1005 such as the information database storing section 1030.

The step of judging whether an epitope binding to the first immunological entity and an epitope binding to the second immunological entity are identical or different based on the structural similarity can also be executed with a program stored in the program storing section 1040, or a software program installed in the external storage device 1005 by inputting various instructions (commands) via the input device 1009 or by receiving commands via the communication I/F, communication device 1011, or the like. The resulting judgment can be outputted through the output device 1007 or stored in the external storage device 1005 such as the information database storing section 1030.

The data or calculation result or information obtained via the communication device 1011 or the like is written and updated immediately in the database storing section 1030. Information attributed to samples subjected to accumulation can be managed with an ID defined in each master table by managing information such as each of the sequences in each input sequence set and each genetic information ID of a reference database.

The above calculation result can be associated with known information such as a disease, disorder, or biological information and stored in the database storing section 1030. Such association can be performed directly to data available through a network (Internet, Intranet, or the like) or as a link to the network.

A computer program stored in the program storing section 1040 is configured to use a computer as the above processing system, e.g., a system for performing the process of, for example, various classifications, division, three-dimensional structure modeling, superimposition, calculation or processing of structural similarity, defining of identical residues, comparison and determination, or the like. Each of these functions is an independent computer program, a module thereof, or a routine, which is executed by the CPU 1001 to use a computer as each system or device. It is assumed hereinafter that each function in each system cooperates to constitute each system.

In one aspect, the present invention provides a method for analyzing an epitope of a subject or a cluster thereof using a database, and/or administering diagnosis or therapy based on a diagnostic result. This method and methods comprising one or more additional characteristics described herein are called “epitope cluster analysis methods” herein. A system materializing the repertoire analysis method of the invention is also called “epitope cluster analysis system of the invention”.

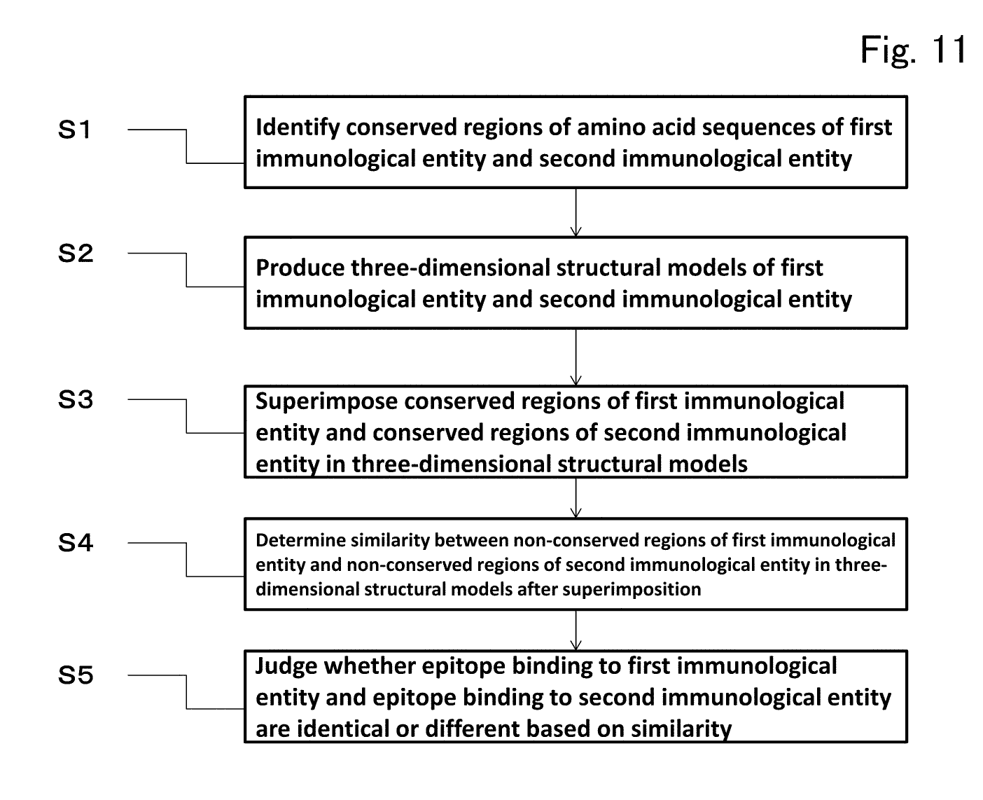

The aforementioned steps are further described with reference to FIG. 11 in addition to FIG. 10.

In S1 (step (1)), amino acid sequences of a first immunological entity and a second immunological entity are provided, and conserved regions (e.g., framework region) of the sequences are identified while other regions such as non-conserved regions (e.g., complementarity-determining region (CDR)) are identified as needed. A sequence is decomposed into conserved regions and non-conserved regions as needed. This can be data stored in the external storage device 1005, but can be generally obtained as a publicly available database through the communication device 1011. Alternatively, this can be inputted using the input device 1009 and recorded in the RAM 1003 or external storage device 1005 as needed. A database comprising sequence information of an immunological entity is provided herein. Sequence information can also be obtained by determining the sequence of an actually obtained sample. Sequence information can be obtained by isolating RNA or DNA from tumor and healthy tissue, and poly A+ RNA from each tissue, to prepare cDNA, and sequencing the cDNA using a standard primer. Such a technology is well known in the art. Full or partial sequencing of the genome of a patient is also well known in the art. High throughput DNA sequencing methods are known in the art, including, for example, systems of the MiSeqg™ series using the Illumina® sequencing technology. This uses a large scale parallel SBS methodology to generate a high quality DNA sequence with several billion bases in one process. Alternatively, an amino acid sequence of an antibody can be determined by mass spectrometry. The portion materializing S1 in the system of the invention is also called a conserved region identifying unit.

In step S2 (step (2)), three-dimensional structure models of a first immunological entity and a second immunological entity are produced. In one specific embodiment, three-dimensional structure models of a conserved region (e.g., framework region) and a non-conserved region (e.g., CDR) are produced for each of the first immunological entity and second immunological entity. In this regard, a three-dimensional model produced based on an amino acid sequence by using, for example, a three-dimensional structure modeling software, is inputted by using the input device 1009 or via the communication device 1011. In this regard, a device that receives amino acid sequence (primary sequence) information of a first immunological entity and a second immunological entity and analyze the gene sequence therefrom, which is also provided in S1, can be connected. Alternatively, such information can be obtained by actually sequencing the amino acid sequence or nucleic acid sequence of an immunological entity such an antibody that has been actually obtained. Such a connection to a gene sequence analysis device can be made through the system bus 1020 or through the communication device 1011. In this regard, trimming and/or extraction of a suitable length can be performed as needed. Such processing is performed by the CPU 1001. A program for three-dimensional modeling can be provided through an external storage device, communication device, or input device. The portion materializing S2 in the system of the invention is also called a three-dimensional structure model producing unit.

S3 (step (3)) performs the superimposition. In this regard, the conserved regions (e.g., framework regions) of the first immunological entity are superimposed with the conserved regions (e.g., framework regions) of the second immunological entity, which were identified or decomposed in S1, based on the three-dimensional structure modeling produced in S2. Upon superimposition, specific processing such as matrix diagonalization or minimalization of root mean square deviation using singular value decomposition can be applied. For such superimposition, data obtained via the communication device 1011 or the like or obtained in S2 is processed. The CPU 1001 performs such processing. A program for the execution thereof can be provided via the external storage device, communication device, or input device. The portion materializing S3 in the system of the invention is also called a superimposing unit.

In S4 (step (4)), similarity (e.g., structural similarity, sequence similarity, or the like) between the first immunological entity and the second immunological entity is determined in the three-dimensional structure models after the superimposition in S3. In this regard, similarity of a non-conserved region (e.g., CDR) is typically determined, and used in comparison and determination of an epitope in S5. This process is also performed by the CPU 1001. A program for the execution thereof can be provided via the external storage device, communication device, or input device. In this regard, in a preferred embodiment, identical residues can be defined using alignment or the like. Defining of identical residues is also performed by the CPU 1001. Structural similarity is also computed by the CPU 1001. These programs can also be provided via the external storage device, communication device, or input device. Results can be stored in the RAM 1003 or the external storage device 1005. A program for such processing can also be provided via the external storage device, communication device, or input device. A portion materializing S4 in the system of the invention is also called a similarity determining unit.

In S5 (step (5)), it is judged whether an epitope binding to the first immunological entity and an epitope binding to the second immunological entity are identical or different based on the similarity (e.g., structural similarity, sequence similarity, or the like) obtained in S4. Similarity is compared to judge whether an epitope binding to the first immunological entity and an epitope binding to the second immunological entity are identical (similar to the extent of belong to an identical cluster) or different, which is also performed by the CPU 1001. The program for this process can also be provided via the external storage device, communication device, or input device. Similarity is judged and then the epitope is deemed to be in an identical cluster, or a different cluster can be generated. Such processing is also performed by the CPU 1001. A program for such processing can also be provided via the external storage device, communication device, or input device. A portion materializing S5 in the system of the invention is also called an identity judging unit.

<Composition, Therapy, Diagnosis, Drug, and the Like>

The present invention also comprises, as an embodiment, the aforementioned classified or clustered epitope, polypeptide, immunological entity binder (e.g., antigen; antigen includes peptides comprising an epitope and the like, as well as those comprising a post-translational modification of glycan or the like, nucleic acids such as DNA/RNA, lower molecule) and polypeptide having substantial similarity to an immunological entity binder or cluster. Another preferred embodiment comprises a polypeptide having functional similarity to one of the above. In still another embodiment, the present invention comprises a nucleic acid encoding the aforementioned classified or clustered epitope, polypeptide, immunological entity binder (e.g., antigen), or cluster, and a polypeptide having substantial similarity thereto. Any characteristic that can be employed therein can be any characteristic described in <Epitope clustering technology> herein or a combination thereof, or anything identified, classified, or clustered by said technology.

In one embodiment, the epitope, cluster, or polypeptide comprising the same of the invention can have affinity to an HLA-A2 molecule. Affinity can be determined by a binding assay, epitope recognition limit assay, prediction algorithm, or the like. The epitope, cluster, or polypeptide comprising the same can have affinity to an HLA-B7 molecule, HLA-B51 molecule, or the like.

In another embodiment of the invention, the present invention provides a pharmaceutical composition comprising a polypeptide, including an epitope that has been classified or clustered in the present invention, a cluster or polypeptide comprising the same, and a pharmaceutically acceptable adjuvant, carrier, diluent, excipient, or the like. An adjuvant can be a polynucleotide. A polynucleotide can comprise a dinucleotide. An adjuvant can be encoded by a polynucleotide. An adjuvant can be a cytokine.

In still another embodiment, the present invention relates to a pharmaceutical composition comprising one of the nucleic acids described herein including a nucleic acid encoding a polypeptide comprising an epitope or immunological entity binder (e.g., antigen) that has been classified or clustered in the present invention. Said composition can comprise a pharmaceutically acceptable adjuvant, carrier, diluent, excipient, or the like.

In still another embodiment, the present invention relates to an isolated and/or purified antibody, an antigen binding fragment, or another immunological entity (e.g., a B cell receptor, a fragment of a B cell receptor, a T cell receptor, a fragment of a T cell receptor, a chimeric antigen receptor (CAR), or a cell comprising one or more of them) specifically binding to at least one epitope that has been classified or clustered in the present invention. In another embodiment, the present invention relates to an isolated and/or purified antibody or another immunological entity specifically binding to a peptide-MHC protein complex comprising an epitope that has been classified or clustered in the present invention or any other suitable epitope. An antibody of any of the embodiments can be a monoclonal antibody or a polyclonal antibody. These compositions can comprise a pharmaceutically acceptable adjuvant, carrier, diluent, excipient, or the like.

In still another embodiment, the present invention relates to a T cell receptor (TCR) and/or B cell receptor (BCR) specifically interacting with at least one epitope that has been classified or clustered in the present invention or a fragment thereof, or an isolated protein molecule comprising a binding domain thereof, or TCR and/or BCR repertoire, chimeric antigen receptor (CAR), or a cell comprising one or more of them (e.g., genetically modified T cell comprising a chimeric antigen receptor (CAR) (also referred to as CAR-T cell), or the like) or another immunological entity. In another embodiment, the present invention relates to an isolated and/or purified antibody or another immunological entity specifically binding to a peptide-MHC protein complex comprising an epitope that has been classified or clustered in the present invention or any other suitable epitope. These compositions can comprise a pharmaceutically acceptable adjuvant, carrier, diluent, excipient, or the like.

In still another aspect, the present invention provides a method for identifying a disease, disorder, or a biological condition, comprising the step of associating a carrier of the immunological entity with a known disease, disorder, or biological condition based on a cluster generated by the method of the invention. Alternatively in another aspect, the present invention provides a method for identifying a disease, disorder, or biological condition, comprising evaluating a disease, disorder or biological condition of a carrier of the cluster using one or more clusters generated by the method of the invention. Any characteristic that can be employed therein can be any characteristic described in <Epitope clustering technology> herein or a combination thereof, or anything identified, classified, or clustered by said technology. In this regard, the evaluating can use, but is not limited to, at least one indicator selected from analysis based on a ranking of quantity and/or a ratio of abundance of the plurality of clusters, and analysis studying a certain number of B cells and quantifying whether there is a cell/cluster similar to a BCR of interest thereamong. In still another embodiment, the evaluating is performed using an indicator other than the cluster (e.g., a disease associated gene, a polymorphism of a disease associated gene, an expression profile of a disease associated gene, epigenetics analysis, a combination of TCR and BCR clusters, and the like). By using the present invention, specifically a disease specific gene that is important in the immune system (HLA allele or the like), a polymorphism of a disease associated gene or an expression profile of the gene (RNA-seq or the like), and epigenetics analysis (methylation analysis or the like) can be combined.

In one embodiment, identification of the disease, disorder, or biological condition identifiable by the present invention can be diagnosis, prognosis, pharmacodynamics, and prediction of the disease, disorder, or biological condition, determination of an alternative method, identification of a patient group, safety evaluation, toxicological evaluation, and monitoring thereof.

In another aspect, the present invention provides a method for evaluating a biomarker, comprising the step of evaluating the biomarker used as an indicator of a disease, disorder, or biological condition using one or more epitopes identified or classified, or clusters refined, by the present invention. Alternatively, the present invention provides a method for identifying a biomarker, comprising the step of determining the biomarker or association with a disease, disorder, or biological conditions using one or more epitopes identified or classified, or clusters refined, by the present invention. In this regard, the following methodology can be used for the method for identifying a biomarker. For example, the presence, size, share, or the like of a cluster of interest of B cell repertoire read by a sequencer can be identified and used as a marker.

In still another embodiment, the present invention relates to a host cell expressing a recombinant construct described herein comprising a construct encoding an epitope that has been classified or clustered in the present invention, cluster, or a polypeptide comprising the same. A host cell can be a dendritic cell, macrophage, tumor cell, tumor derived cell, bacteria, fungus, protozoa, or the like. This embodiment also provides a pharmaceutical composition comprising such a host cell and a pharmaceutically acceptable adjuvant, carrier, diluent, excipient, or the like.

In another aspect, the present invention provides a composition for identifying the biological information, comprising an epitope or an antigen or an immunological entity binder comprising the same identified based on the present invention. Alternatively, the present invention provides a composition for diagnosing the disease, disorder, or biological condition, comprising an epitope or an antigen or an immunological entity binder comprising the same identified based on the present invention. Any characteristic that can be employed therein can be any characteristic described in <Epitope clustering technology> herein or a combination thereof, or anything identified, classified, or clustered by said technology.

In another aspect, the present invention provides a composition for diagnosing the disease, disorder, or biological condition, comprising a substance targeting an immunological entity to an epitope identified based on the present invention. Alternatively, the present invention provides a composition for diagnosing the disease, disorder, or biological condition, comprising an epitope or an antigen or an immunological entity binder comprising the same identified by the present invention. Any characteristic that can be employed therein can be any characteristic described in <Epitope clustering technology> herein or a combination thereof, or anything identified, classified, or clustered by said technology. Therefore, examples of the immunological entity include an antibody, an antigen binding fragment of an antibody, a T cell receptor, a fragment of a T cell receptor, a B cell receptor, a fragment of a B cell receptor, a chimeric antigen receptor (CAR), a cell comprising one or more of them (e.g., T cell comprising a chimeric antigen receptor (CAR)), and the like.

In still another embodiment, the present invention provides a composition for treating or preventing the disease, disorder, or biological condition, comprising an immunological entity to an epitope identified based on the present invention. Any characteristic that can be employed therein can be any characteristic described in <Epitope clustering technology> herein or a combination thereof, or anything identified, classified, or clustered by said technology. Further, immunological entities that can be used include, but are not limited to, an antibody, an antigen binding fragment, a chimeric antigen receptor (CAR), a T cell comprising a chimeric antigen receptor (CAR), and the like.

In another aspect, the present invention provides a composition for treating or preventing the disease, disorder, or biological condition, comprising a substance targeting an immunological entity to an epitope identified based on the present invention. Any characteristic that can be employed therein can be any characteristic described in <Epitope clustering technology> herein or a combination thereof, or anything identified, classified, or clustered by said technology. Examples of the substance that can be used include, but are not limited to, a peptide, polypeptide, protein, nucleic acid, sugar, lower molecule, macromolecule, metal ion, and a complex thereof.

In another aspect, the present invention provides a composition for treating or preventing the disease, disorder, or biological condition, comprising an epitope or immunological entity binder (e.g., antigen) comprising the same identified based on the present invention. Any characteristic that can be employed therein can be any characteristic described in <Epitope clustering technology> herein or a combination thereof, or anything identified, classified, or clustered by said technology.

In still another embodiment, the present invention relates to a vaccine or an immunotherapeutic composition comprising at least one constituent component such as an epitope that has been classified or clustered in the present invention, cluster comprising the epitope, immunological entity binder (e.g., antigen) or polypeptide comprising the epitope, composition described above or herein, or T cell or host cell described above or herein.

The present invention also relates to a diagnostic method or therapeutic method. The method can comprise a step of administering a pharmaceutical composition such as an immunotherapeutic composition or a vaccine comprising a component disclosed herein to an animal. Examples of administration can include transdermal, intranodular, perinodal, oral, intravenous, intradermal, intramuscular, intraperitoneal, mucosal, aerosol inhalation, instillation delivery modes. The method can further comprise the step of assaying to determine a characteristic indicating a state of a target cell. The method can further comprise a first assaying step and a second assaying step, wherein the first assaying step is performed before a step of administering a therapeutic drug or the like, and the second assaying step is performed after the step of administering a therapeutic drug or the like. In this case, the method can further comprise a step of comparing a characteristic determined by the first assaying step with a characteristic determined by the second assay step, thereby obtaining a result. The result can be, for example, an indication of an immune response, decrease in the target cell count, decrease in the mass or size of tumor comprising a target cell, decrease in the number or concentration of intracellular parasite infected target cells or the like. The result can be judged based on an epitope that has been classified, identified, or clustered by the method of the invention.

The present invention relates to a method for making a passive/adoptive immunotherapeutic drug with an epitope that has been classified or clustered in the present invention, cluster comprising the epitope, or immunological entity binder (e.g., antigen) or polypeptide comprising the epitope. The method can comprise combining a T cell or host cell described in other parts herein with a pharmaceutically acceptable adjuvant, carrier, diluent, excipient, or the like. A buffer, binding agent, blasting agent, diluent, flavoring agent, lubricant, or the like can be included as the excipient.

In one aspect, the present invention relates to a method for diagnosing a disorder, disease, or biological condition using an epitope that has been classified or clustered in the present invention, cluster comprising the epitope, immunological entity binder (e.g., antigen) or polypeptide comprising the epitope, or the like. The method can comprise contacting subject tissue with at least one constituent component comprising, for example, a T cell, host cell, antibody, and protein, including any one of the components described above or in other parts herein, and diagnosing a disease based on a characteristic of the tissue or constituent component. The contacting step can be performed, for example, in vivo or in vitro. The present invention further comprises a step of identifying a classified epitope. Such an identification step comprises determining of the structure thereof as well as, but not limited to, determining an amino acid sequence, identifying a three-dimensional structure, identifying of another structure, identifying a biological function, or the like.

In still another embodiment, the present invention relates to a method for making a vaccine. This method can comprise combining at least one constituent component including an epitope, composition, construct, T cell, and host cell including any of the components described in other parts herein with a pharmaceutically acceptable adjuvant, carrier, diluent, excipient, or the like. In another embodiment, the present invention can evaluate or improve a vaccine using the clustering and classification method of the invention and an epitope, immunological entity or immunological entity binder identified therewith. The present invention can also evaluate and/or generate or improve a biomarker using an identified epitope or an immunological entity binder comprising the same or the cluster itself. In this regard, “improve” means providing a methodology that can more appropriately evaluate neutralizing antibody production upon vaccination by identifying a cluster whose antibody titer is desirably increased by clustering, where the methodology is for improving vaccine performance by being performed in parallel with a normal experiment. Examples of “evaluation” of a biomarker include a method for at first identifying a cluster (e.g., cluster correlated with a state of a disease) that can be a biomarker itself and investigating whether a more simple experimentation (e.g., can be performed using an ELISA binding assay or the like) is able to suitably follow an expected change in the cluster. Such a case presumes that the cluster itself can function as a marker, but this can also be made in the same manner (to reflect information of the cluster).

The present invention also provides a composition for evaluating a vaccine for treating or preventing a disease, disorder, or biological condition, comprising an immunological entity to an epitope identified based on the present invention. For such evaluation, Example 6 and the like describes an example of influenza viruses, which can be applied. In another aspect, the present invention relates to a method for treating or preventing a disease using an epitope that has been classified or clustered in the present invention, cluster comprising the epitope, immunological entity binder (e.g., antigen) or polypeptide comprising the epitope, or the like. The method can comprise combining a therapeutic method of an animal comprising administering a vaccine or immunotherapeutic composition described in other parts herein to the animal with at least one therapeutic mode including, for example, radiation therapy, chemotherapy, biochemical therapy, and surgery.

The present invention also relates to a vaccine or immunotherapeutic product comprising an epitope that has been classified or clustered in the present invention, cluster comprising the epitope, immunological entity binder (e.g., antigen) or polypeptide comprising the epitope, or the like. A still another embodiment relates to an isolated polynucleotide encoding a polypeptide described in other parts herein. Another embodiment relates to a vaccine or immunotherapeutic product comprising such a polynucleotide. A polynucleotide can be a DNA, RNA, or the like.

In one embodiment, the present invention also relates to a kit comprising a delivery device and any one of the embodiments described in other parts herein. A delivery device can be a catheter, syringe, internal or external pump, reservoir, inspiratory, microinjector, patch, or any other similar device suitable for any route of delivery. As discussed above, a kit can also comprise any one of the embodiments disclosed herein in addition to a delivery device. For example, a kit can comprise, but not limited to, an isolated epitope, polypeptide, cluster, nucleic acid, immunological entity binder (e.g., antigen), pharmaceutical composition comprising any one of the above, antibody, T cell, T cell receptor, epitope-MHC complex, vaccine, immunotherapeutic drug, or the like. A kit can also comprise an item such as a detailed user manual or any other similar item.

A particularly desirable strategy for including an epitope and/or epitope cluster in a vaccine or a pharmaceutical composition is disclosed in U.S. patent application Publication Ser. No. 09/560,465 entitled “EPITOPE SYNCHRONIZATION IN ANTIGEN PRESENTING CELLS” filed on Apr. 28, 2000.

The vaccine that can be used in the present invention comprises an epitope or an immunological entity binder (e.g., antigen) at a concentration effective to present an epitope that has been classified, identified, or clustered in the present invention. Preferably, the vaccine of the invention can comprise a plurality of the epitope of the invention or cluster thereof in combination with any one or more immunological epitopes. The vaccine formulation of the invention comprises a peptide and/or nucleic acid at a concentration that is sufficient to present an epitope to a target. The formulation of the invention preferably comprises an epitope or a peptide comprising the same at a total concentration of about 1 μg to 1 mg/(100 μl of vaccine preparation). Conventional dosage and dosing related to a peptide vaccine and/or nucleic acid vaccine can be used with the present invention. Such a dosing regimen is thoroughly understood in the art. In one embodiment, a single dose for adults is suitably about 1 to 5000 μl of composition, which is administered as a single or multiple dose, such as two, three, four or more doses separated in 1 week, 2 weeks, 1 month, or more. The vaccine of the invention can comprise a recombinant organism such as a virus, bacteria, or protozoa genetically engineered to express an epitope in a host.

The vaccine, composition, and method of the invention can blend an adjuvant to a formulation to enhance the performance of the vaccine. Specifically, an adjuvant can be designed to enhance the delivery and intake of an epitope. Adjuvants intended by the present invention are known to those skilled in the art. Examples thereof include GMCSF, GCSF, IL-2, IL-12, BCG, tetanus toxoid, osteopontin, and ETA-1.

The vaccine or the like of the invention can be administered by any suitable method. The vaccine of the invention is administered to a patient in a mode consistent with a standard vaccine delivery protocol known in the art. Examples of epitope delivery methods include, but are not limited to, transdermal, intranodular, perinodal, oral, intravenous, intradermal, intramuscular, intraperitoneal, and mucosal administration, including delivery by injection, instillation, or inhalation. Particularly useful methods of vaccine delivery for inducing a CTL response are disclosed in AU Patent No. 739189 published on Jan. 17, 2002, U.S. patent application Publication Ser. No. 09/380,534 filed on Sep. 1, 1999, and partially simultaneously pending U.S. patent application Publication Ser. No. 09/776,232 filed on Feb. 2, 2001, which are incorporated herein by reference.

In one embodiment, the present invention can also comprise a protein, antibody, cell that can express them, specific B cell and T cell, or the like, which specifically binds to an epitope or an immunological entity binder (e.g., antigen) at a concentration effective to present an epitope that has been classified, identified, or clustered in the present invention. These reagents are in a form of an immunoglobulin, i.e., a polyclonal serum or monoclonal antibody whose production method is well known in the art. Production of mAb having specificity related to a peptide-MHC molecule complex is known in the art (Aharoni et al. Nature 351: 147-150, 1991 and the like). General construct and use are also discussed in U.S. Pat. No. 5,830,755 entitled T CELL RECEPTORS AND THEIR USE IN THERAPEUTIC AND DIAGNOSTIC METHODS.

In one embodiment, one of epitope and an immunological entity binder (e.g., antigen) comprising the same at a concentration effective to present an epitope that has been classified, identified, or clustered in the present invention can be bound to an enzyme, radioactive chemical substance, fluorescent tag, and toxin for use in diagnosing (imaging or other detection), monitoring, and treating an epitope associated pathogenic state. Therefore, a toxin conjugate can be administered to kill tumor cells, and a radiolabel can facilitate imaging of epitope positive tumor, and an enzyme conjugate can be used in an ELISA-like assay to diagnose cancer and confirm epitope expression in biopsy tissue. In still another embodiment, T cells described above can be administered to a patient as an adoptive immunotherapy after proliferation achieved by stimulation with a cytokine and/or epitope.

In another embodiment, the present invention provides a complex of an epitope that has been classified, identified, or clustered in the present invention and MHC or a peptide-MHC complex as an epitope. In a particularly suitable embodiment, a complex can be a soluble multimer protein described in U.S. Pat. No. 5,635,363 (tetramer) or U.S. Pat. No. 6,015,884 (Ig-dimer). Such a reagent is useful for detecting and monitoring a specific T cell response and purifying said T cell.

In another embodiment, an epitope that has been classified, identified, or clustered in the present invention can be used to perform a functional assay, evaluate endogenous immunity level or a response to immunological stimulation (e.g., vaccine), and monitor the immune state due to the path of therapy and the disease. Except when measuring an endogenous immunity level, each of these assays can presume a preliminary step for immunity in vivo or in vitro depending on the nature of the problem to be addressed. Such immunity can be performed using various embodiments of the invention, or immunogen in other forms that can induce the same immunity. Except for tetramer/Ig-dimer analysis and PCR that can detect the expression of homologous TCRs, these assays can generally benefit from the step of in vitro antigenic stimulation that can suitably use various aforementioned embodiments of the invention in order to detect a specific functional activity (can be directly detected for a high cytolytic response). Finally, detection of cytolytic activity requires epitope presenting target cells, which can be produced using various embodiments of the invention. The specific embodiment selected for any specific step is dependent on the problem to be addressed, ease of use, cost, or the like, but the advantage of one embodiment over another embodiment related to any specific pair of circumstances is evident to those skilled in the art.

Such a functional assay can use an activation step or a reading step or both in a form of an epitope of the invention or a complex with an MHC molecule. Two categories of assays, assay for measuring a response of a cell pool and an assay for measuring a response of individual cells, can be practiced among the many assays of T cell functions known in the art (detailed procedures can be found in standard immunological reference documents such as Current Protocols in Immunology 1999 John Wiley & Sons Inc., N.Y). The former can measure the overall strength of responses, while the latter can determine the relative frequency of responsive cells. Examples of assay for measuring an overall response include cytotoxic assay, ELISA, and proliferation assay for detecting cytokine secretion. Examples of the assay for measuring a response of individual cells include limiting dilution analysis (LDA), ELISPOT, flow cytometric detection of unsecreted cytokines (described in U.S. Pat. Nos. 5,445,939, 5,656,446, and 5,843,689, and reagents therefor are sold by Becton, Dickinson & Company under the product name “FASTIMMUNE”), detection of specific TCR with a tetramer or Ig-dimer as discussed and cited above (see also Yee, C. et al. Current Opinion in Immunology, 13: 141-146, 2001).

The present invention can be provided as a kit. As used herein, “kit” refers to a unit providing parts to be provided (e.g., test drug, diagnostic drug, therapeutic drug, antibody, label, user manual, and the like) which are generally separated into two or more segments. Such a kit form is preferred when providing a composition, which should not be provided in a mixed state for stability or the like and is preferably used by mixing immediately prior to use. Such a kit preferably comprises an instruction or manual describing how the provided portions (e.g., test drug, diagnostic drug, or therapeutic drug) are used or how a reagent should be processed. When a kit is used as a reagent kit herein, the kit generally comprises an instruction or the like describing the method of use of a test drug, diagnostic drug, therapeutic drug, antibody, or the like.

In this manner, in still another aspect of the invention, the present invention relates to a kit having (a) a container comprising the pharmaceutical composition of the invention in a solution or lyophilized form, (b) optionally a second container comprising a diluent or reconstitution solution for the lyophilized formulation, and (c) optionally a manual for the (i) use of the solution or (ii) reconstitution and/or use of the lyophilized formulation. The kit further has one or more of (iii) a buffer, (iv) a diluent, (v) a filter, (vi) a needle, or (v) a syringe. The container is preferably a bottle, vial, syringe, or test tube, and the container may be a multi-purpose container. The pharmaceutical composition is preferably lyophilized.

The kit of the invention preferably has a manual for the lyophilized formulation of the invention and reconstitution and/or use thereof in a suitable container. Examples of the suitable container include a bottle, vial (e.g., dual chamber vial), syringe (dual chamber syringe or the like), and test tube. The container can be made of various materials such as glass or plastic. Preferably, the kit and/or container comprises a manual showing the method of reconstitution and/or use on the container or accompanying the container. For example, the label thereof can have an explanation showing that the lyophilized formulation is reconstituted to have the concentration of the above peptide. The label can further have an explanation showing that the formulation is useful for, or is for subcutaneous injection.

The container of the formulation can be a multi-purpose vial that can be used for repeated dosing (e.g., 2 to 6 dosing). The kit can further have a second container having a suitable diluent (e.g., sodium bicarbonate solution).

The final peptide concentration of a reconstituted formulation made by mixing the diluent and the lyophilized formulation is preferably at least 0.15 mg/mL/peptide (when=75 μg, 0.5 ml) and preferably 3 mg/mL/peptide (when=1500 μg, 0.5 ml) or less. The kit can further comprise other materials (including other buffer, diluent, filter, needle, syringe, and user manual inserted into the package) that are desirable from the commercial viewpoint or user viewpoint.

The kit of the invention can have a single container comprising a formulation of the pharmaceutical composition of the invention with or without other constituent elements (e.g., other compounds or pharmaceutical composition of such other compounds) or have another container for each constituent element.

The kit of the invention preferably comprises a formulation of the invention which is packaged for use as a combination with a second compound (adjuvant (e.g., GM-CSF), chemotherapeutic agent, naturally-occurring product, hormone or antagonist, other drug, or the like) or a pharmaceutical composition thereof. Constituent elements of the kit can be constituents made in advance as a complex, or each constituent element placed in separate containers until administration to a patient. The constituent elements of the kit can be provided as one or more liquid solutions, preferably and aqueous solution, and more preferably sterilized aqueous solution. The constituent elements of the kit can also be provided as a solid. Preferably, a suitable solution provided in separate different container can be added there to convert the solid to a liquid.

A container of a therapeutic kit can be a vial, test tube, flask, bottle, syringe, or any other means for sealing a solid or liquid. Generally, the kit comprises a second vial or another container when there are a plurality of constituent elements so that the elements can be administered separately. The kit can also comprise another container for a pharmaceutically acceptable solution. Preferably, a therapeutic kit comprises an instrument (e.g., one or more of needle, syringe, eye dropper, pipette, and the like) enabling the administration of the agent of the invention, which is a constituent element of the kit.

The pharmaceutical composition of the invention is suitable for administrating the peptide through any acceptable route, such as oral (enteral), nasal, ocular, subcutaneous, intradermal, intramuscular, intravenous, or transdermal route. Preferably, the administration is subcutaneous administration and most preferably intradermal administration. The pharmaceutical composition can be administered by an injection pump.

As used herein, “instruction” is a document with an explanation of the method of use of the present invention for a physician or other users. The instruction describes a detection method of the invention, how to use a diagnostic drug, or a description instructing administration of a drug or the like. Further, an instruction may have a description instructing oral administration, or administration to the esophagus (e.g., by injection or the like) as the site of administration. The instruction is prepared in accordance with a format specified by a regulatory authority of the country in which the invention is practiced (e.g., Ministry of Health, Labour and Welfare in Japan, Food and Drug Administration (FDA) in the U.S., or the like), with an explicit description showing approval by the regulatory authority. The instruction is a so-called label or package insert, and is generally provided in, but not limited to, paper media. The instructions may also be provided in a form such as electronic media (e.g., web sites provided on the Internet or emails).

As used herein, “or” is used when “at least one or more” of the listed matters in the sentence can be employed. When explicitly described herein as “within the range” of “two values”, the range also includes the two values themselves.

(General Technology)

Any molecular biological methodologies, biochemical methodologies, microbiological methodologies, and bioinformatics used herein that is known in the art, well known, or conventional can be used.

Reference literatures such as scientific literatures, patents, and patent applications cited herein are incorporated herein by reference to the same extent that the entirety of each document is specifically described.

As described above, the present invention has been described while showing preferred embodiments to facilitate understanding. The present invention is described hereinafter based on Examples. The above descriptions and the following Examples are not provided to limit the present invention, but for the sole purpose of exemplification. Thus, the scope of the present invention is not limited to the embodiments and Examples specifically described herein and is limited only by the scope of claims.

### EXAMPLES

The Examples are described hereinafter. When necessary, all experiments were conducted in compliance with the guidelines approved by the ethics committee of the Osaka University in the following Examples. For reagents, the specific products described in the Examples were used. However, the reagents can be substituted with an equivalent product from another manufacturer (Sigma-Aldrich, Wako Pure Chemical, Nacalai Tesque, R & D Systems, USCN Life Science INC, or the like).

### Example 1: Example Using an HIV Antibody

This Examples shows that an anti-HIV antibody can be clustered by epitopes even when there are a very large amount of non-anti-HIV antibodies by using the methodology proposed herein.

This Example first selected out human derived antibody-antigen complexes that are peptides with an antigen length of 6 residues or more from structures registered in PDB (Protein Data Bank) and then reviewed the following two data sets.

(HIV Sets)

270 human derived anti-HIV antibodies were obtained from the PDB database. The names of the antibodies are listed below (In the Table, the first 4 digits indicate the PDB ID, 5th to 7th digits indicate heavy chain, light chain, and antigen chain IDs, respectively).

TABLE 1-1

| | | | | | |
|---|---|---|---|---|---|
| 1n0xHLP | 3h3pIMT | 5cinHLP | 3macHLA | 4olxHLG | 5a8hRQM |
| 1n0xKMR | 3idgBAC | 1g9mHLG | 3ngbBCA | 4olyHLG | 5acoGJC |
| 1q1jHLP | 3idjBAC | 1g9nHLG | 3ngbEFD | 4olzHLG | 5acoHLA |
| 1q1jIMQ | 3idmBAC | 1gc1HLG | 3ngbHLG | 4om0HLG | 5acoIKD |
| 1tjgHLP | 3idnBAC | 1rzjHLG | 3ngbJKI | 4om1HLG | 5c0sHLA |
| 1tjhHLP | 3mlrHLP | 1rzkHLG | 3p30HLA | 4p9hHLG | 5c7kABC |
| 1tjiHLP | 3mlsHLP | 1yylHLG | 3q1sHLI | 4r2gDCO | 5c7kEFD |
| 1tzgHLP | 3mlsIMQ | 1yylRQP | 3ru8HLX | 4r2gJIK | 5cezDEB |
| 1tzgIMQ | 3mlsJNR | 1yymHLG | 3se8HLG | 4r2gNMA | 5cezHLG |
| 1u8hBAC | 3mlsKOS | 1yymRQP | 3se9HLG | 4r2gQPE | 5esvABE |
| 1u8iBAC | 3mltBAC | 2b4cHLG | 3u2sABC | 4rfoHLG | 5esvCDF |
| 1u8jBAC | 3mltHLP | 2cmrHLA | 3u2sHLG | 4rqsDCG | 5esvHLG |
| 1u8kBAC | 3mluHLP | 2i5yHLG | 3u4eABJ | 4rwyHLA | 5eszABC |
| 1u8lBAC | 3mlvHLP | 2i5yRQP | 3u4eHLG | 4rx4ADE | 5eszHLG |
| 1u8mBAC | 3mlvNMQ | 2i60HLG | 3u7yHLG | 4rx4HLG | 5f6jBAG |
| 1u8nBAC | 3mlwHLP | 2i60RQP | 4dqoHLC | 4s1qHLG | 5f6jHFE |
| 1u8oBAC | 3mlwIMQ | 2nxyDCA | 4dvrHLG | 4s1rHLG | 5f96HLG |
| 1u8pBAC | 3mlxHLP | 2nxzDCA | 4h8wHLG | 4s1sHLG | 5f9oHLG |
| 1u8qBAC | 3mlxIMQ | 2ny0DCA | 4i3rHLG | 4tvpDEB | 5f9wBCA |
| 1u91BAC | 3mlyHLP | 2ny1DCA | 4i3sHLG | 4tvpHLG | 5f9wHLG |
| 1u92BAC | 3mlyIMQ | 2ny2DCA | 4j6rHLG | 4xmpHLG | |
| 1u93BAC | 3mlzHLP | 2ny3DCA | 4janABI | 4xnyHLG | |
| 1u95BAC | 3moaHLP | 2ny4DCA | 4janHLG | 4xnzBCA | |
| 2b0sHLP | 3mobHLP | 2ny5HLG | 4jb9HLG | 4xnzEFD | |
| 2b1aHLP | 3ujiHLP | 2ny6DCA | 4jdtHLG | 4xnzHLG | |
| 2b1hHLP | 3ujjHLP | 2ny7HLG | 4jkpHLG | 4xvsHLG | |
| 2f5bHLP | 4g6fBDF | 2qadDCA | 4jm2ABE | 4xvtHLG | |
| 2fx8HLP | 4g6fHLP | 2qadHGE | 4jm2DCE | 4yblBCA | |
| 2fx8IMQ | 4hpoHLP | 3hi1BAJ | 4jpvHLG | 4yblHLG | |
| 2fx8JNR | 4hpyHLP | 3hi1HLG | 4jpwHLG | 4yc2BCA | |
| 2fx8KOS | 4m1dHLP | 3idxHLG | 4khtHLA | 4yc2HLG | |
| 2fx9HLP | 4m1dIMQ | 3idyBCA | 4khxHLA | 4ydiHLG | |
| 2fx9IMQ | 4nghHLP | 3idyHLG | 4lspHLG | 4ydjABI | |

TABLE 1-2

| | | | | |
|---|---|---|---|---|
| 2p8lBAC | 4nhcHLP | 3j5mDCA | 4lsqHLG | 4ydjHLG |
| 2p8mBAC | 4nrxABC | 3j5mHGE | 4lsrHLG | 4ydkHLG |
| 2p8pBAC | 4nrxHLP | 3j5mLKI | 4lssHLG | 4ydlBCA |
| 2pw1BAC | 4risHLP | 3j70ABD | 4lstHLG | 4ydlHLG |
| 2qscHLP | 4u6gABC | 3j70MNP | 4lsuHLG | 4ye4HLG |
| 3d0lBAC | 4u6gHLP | 3j70RSU | 4lsvHLG | 4yflFIE |
| 3d0vBAC | 4xawHLP | 3jwdHLA | 4m62HLS | 4yflHLG |
| 3droBAP | 4xbeHLP | 3jwdPOB | 4m62IMT | 4ywgHLG |
| 3drqBAC | 4xc1HLP | 3jwoHLA | 4m8qABS | 4ywgIMQ |
| 3drtBAC | 4xc3HLP | 3levHLA | 4m8qHLC | 5a7xDCA |
| 3egsBAC | 4xcfHLP | 3lh2HLS | 4ncoDCA | 5a7xHGE |
| 3fn0HLP | 4xmkHLP | 3lh2IMT | 4ncoHGE | 5a7xLKI |
| 3ghbHLP | 4xmkIMQ | 3lh2JNU | 4ncoLKI | 5a8hDCA |
| 3ghbIMQ | 4xmkJNR | 3lh2KOV | 4nzrHLM | 5a8hFEA |
| 3gheHLP | 4ydvBAQ | 3lhpHLS | 4oluHLG | 5a8hJIG |
| 3go1HLP | 4ydvHLP | 3lhpIMT | 4olvHLG | 5a8hLKG |
| 3h3pHLS | 5cilHLP | 3ma9HLA | 4olwHLG | 5a8hPOM |

Antibodies with very close sequence homology (90% or greater) were excluded in advance using a program called cd-hit (available from J. Craig Venter Institute). In this regard, only antibodies with sequence homology of less than 90% for both heavy chain and light chain were kept. For antibodies with an antibody structure comprising not only variable domains but also constant domains, those were also included.

The three-dimensional structure of each antibody is registered in PDB. The epitope can also be found from the structural data.

Furthermore, if only one antibody is deemed to recognize an identical epitope, the antibody was excluded.

The IDs of selected structures in PDB are the following.

| | | | | | |
|---|---|---|---|---|---|
| 2b1hHLP | 3lh2HLS | 3mlrHLP | 3mlwHLP | 3se8HLG | 3se9HLG |
| 4j6rHLG | 4janABI | 4jb9HLG | 4jpvHLG | 4jpwHLG | 4lspHLG |
| 4lsuHLG | 4m62HLS | 4rwyHLA | 4tvpHLG | 4xcfHLP | 4xmpHLG |
| 4xnyHLG | 4xvtHLG | 4ydiHLG | 4ydkHLG | 4ydlBCA | 4yflFIE |
| 5cezHLG | 5f96HLG | 5f9oHLG | | | |

### (Non-HIV Set)

275 human derived non-anti-HIV antibodies (obtained from PDB database; the explanatory note is the same as Table 1)

TABLE 2-1

| | | | | |
|---|---|---|---|---|
| 1adqHLA | 2gr0TSU | 3g6dHLA | 3u30FED | 4fp8JNC |
| 1bvkBAC | 2qr0XWV | 3gbnHLB | 3uluDCA | 4fp8KOD |
| 1bvkEDF | 2r56HLA | 3grwHLA | 3uluFEA | 4fqiHLB |
| 1deeDCG | 2r56IMB | 3h0tBAC | 3uluHLA | 4fqjHLA |
| 1deeFEH | 2uziHLR | 3h42HLB | 3ulvDCA | 4fqkEFC |
| 1h0dBAC | 2vh5HLR | 3hi6HLA | 3ulvFEA | 4fqkHLA |
| 1hezBAE | 2vxqHLA | 3hi6XYB | 3ulvHLA | 4fqrabA |
| 1hezDCE | 2vxsHLB | 3hmxHLA | 3w9eABC | 4fqrcdC |
| 1i9rHLA | 2vxsIMA | 3iywHLA | 3wd5HLA | 4fgrefE |
| 1i9rKMB | 2vxsJND | 3iywKMC | 3whe12H | 4fqrghG |
| 1i9rXYC | 2vxsKOC | 3k2uHLA | 3whe34I | 4fqrijI |
| 1ikfHLC | 2wubHLA | 3kr3HLD | 3whe56J | 4fqrklK |
| 1iqdBAC | 2wubRQC | 3l5wBAJ | 3whe78K | 4fqrmnM |
| 1jpsHLT | 2wucHLA | 3l5wHLI | 3whe90L | 4fgropO |
| 1nl0HLG | 2xqbHLA | 3l5xHLA | 3wheMNA | 4fqrqrQ |
| 1uj3BAC | 2xraHLA | 3lzfHLA | 3wheOPB | 4fqrstS |
| 1yy9DCA | 2xtjDBA | 3mnwBAP | 3wheQRC | 4fqruvU |
| 2dd8HLS | 2xwtABC | 3mnzBAP | 3wheSTD | 4fqrwxW |
| 2eizBAC | 2ybrABC | 3mugFEC | 3wheUVE | 4fqyHLB |
| 2eksBAC | 2ybrDEF | 3mugLKI | 3wheWXF | 4g3yHLC |
| 2fecILB | 2ybrGHI | 3mxwHLA | 3wheYZG | 4g6aCDB |
| 2fecJOA | 2yc1ABC | 3n85HLA | 3wlwCDA | 4g6aHLA |
| 2fedCDA | 2yc1DEF | 3nfpABK | 3wlwHLB | 4g6jHLA |
| 2fedEFB | 2yssBAC | 3nfpHLI | 3wsqHLA | 4g6mHLA |
| 2feeILB | 3b2uCDB | 3nh7HLA | 3x3fHLA | 4g7vHLS |
| 2feeJOA | 3b2uFGE | 3nh7IMB | 3ztnHLB | 4g7yHLS |
| 2fjgBAV | 3b2uHLA | 3nh7JNC | 4al8HLC | 4g80ABS |
| 2fjgHLW | 3b2uJKI | 3nh7KOD | 4am0ABS | 4g80CDJ |
| 2fjhBAW | 3b2uNOM | 3npsBCA | 4am0CDT | 4g80EFT |
| 2fjhHLV | 3b2uQRP | 3p0yHLA | 4am0EFQ | 4g80GHI |
| 2h9gBAR | 3b2uTUS | 3p11HLA | 4am0HLR | 4gxuMNA |
| 2h9gHLS | 3b2uWXV | 3pgfHLA | 4cniABD | 4gxuOPC |
| 2hfgHLR | 3b2vHLA | 3r1gHLB | 4cniHLC | 4gxuQRE |

TABLE 2-2

| | | | | |
|---|---|---|---|---|
| 2j6eHLA | 3bdyHLV | 3s35HLX | 4d9qEDB | 4gxuSTG |
| 2j6eIMB | 3be1HLA | 3s36HLX | 4d9qHLA | 4gxuUVI |
| 2oqjBAC | 3bkyHLP | 3s37HLX | 4d9rEDB | 4gxuWXK |
| 2oqjEDF | 3bn9DCB | 3sdyHLB | 4d9rHLA | 4hcrHLA |
| 2oqjHGI | 3bn9FEA | 3skjHLE | 4dagHLA | 4hcrMNB |
| 2oqjKJL | 3c09CBA | 3skjIMF | 4dgvHLA | 4hf5HLA |
| 2oslABQ | 3c09HLD | 3sm5HLA | 4dgyHLA | 4hfuHLA |
| 2oslHLP | 3c2aHLP | 3sm5IMC | 4dkeHLA | 4hg4JKA |
| 2qqkHLA | 3c2aIMQ | 3sm5JNE | 4dkeIMB | 4hg4LMB |
| 2qqlHLA | 3d85BAC | 3so3CBA | 4dkfHLA | 4hg4NOC |
| 2qqnHLA | 3eoaBAJ | 3sobHLB | 4dkfIMB | 4hg4VWG |
| 2qr0BAD | 3eoaHLI | 3sqoHLA | 4dn4HLM | 4hg4XYH |
| 2qr0FEC | 3eobBAJ | 3t2nHLA | 4dtgHLK | 4hhaBAP |
| 2qr0HGJ | 3eobHLI | 3t2nIMB | 4edwHLV | 4hj0CDB |
| 2gr0LKI | 3eyfBAE | 3u0tBAF | 4ersHLA | 4hj0PQA |
| 2qr0NMO | 3eyfDCF | 3u0tDCE | 4fp8HLA | 4hkxABE |
| 2qr0RQP | 3g04BAC | 3u30CBA | 4fp8IMB | 4hs6BAZ |
| 4o4yHLA | 4uu9ABC | 4xx1EGB | 4hs6HLY | 5c7xMNB |
| 4o51BAN | 4uu9HLD | 4xx1HLA | 4hs8HLA | 5c8jABI |
| 4o51DCO | 4uv7HLA | 4xx1MOJ | 4hwbHLA | 5c8jCDL |
| 4o51FEP | 4v1dABC | 4xxdBAC | 4i2xBAE | 5c8jEFJ |
| 4o51HLM | 4v1dDEC | 4xxdEDF | 4i2xDCF | 5c8jGHK |
| 4o58HLA | 4wv1BAC | 4y5vABC | 4i77HLZ | 5cszABE |
| 4o5iMNA | 4wv1EDF | 4y5vDEF | 4idjHLA | 5cszHLD |
| 4o5iOPC | 4xakDEB | 4y5vGHI | 4irzHLA | 5cusHLA |
| 4o5iQRE | 4xakHLA | 4y5xABC | 4j4pCDB | 5cusIMB |
| 4o5iSTG | 4xgzABa | 4y5xDEF | 4j4pHLA | 5cusJNC |
| 4o5lUVI | 4xgzCDc | 4y5xGHI | 4jhwHLF | 5cusKOD |
| 4o5iWXK | 4xgzEFe | 4y5xJKL | 4jznIPK | 5d70HLA |
| 4odxALY | 4xgzGIg | 4y5yABC | 4jzoABJ | 5d71HLA |
| 4odxHBX | 4xgzHLh | 4y5yDEF | 4jzoCFK | 5d72HLA |
| 4ogxHLA | 4xgzJKj | 4yhpCDQ | 4jzoDEI | 5d72MNB |
| 4ogyHLA | 4xgzMNm | 4yhpHLP | 4jzoGHL | 5dumHLA |
| 4ogyMNB | 4xgzOPo | 4yhzHLP | 4k8rDCB | 5dupHLA |

TABLE 2-3

| | | | | |
|---|---|---|---|---|
| 4oqtHLA | 4xgzQRq | 4yk4CBA | 4kroDCA | 5durBDA |
| 4ot1HLA | 4xgzSTs | 4yk4ZYE | 4krpDCA | 5durHLC |
| 4p59HLA | 4xgzUVu | 4ypgBAC | 4kv5EFC | 5e8eBAH |
| 4ps4HLA | 4xgzWXw | 4ypgHLD | 4kv5GKD | 5f45HLA |
| 4qciBAD | 4xh2ABa | 4z5rBAN | 4kv5HLA | 5fgcEBA |
| 4qciHLC | 4xh2CDc | 4z5rKJD | 4kv5JIB | 5fhcABJ |
| 4qhuBAD | 4xh2EFe | 4z5rMLE | 4kvnHLA | 5fhcHLK |
| 4qhuHLC | 4xh2GIg | 4z5rQPG | 4kxzHLA | 5i5kHLB |
| 4ravABE | 4xh2HLh | 4z5rSRH | 4kxzJIB | 5i5kXYA |
| 4ravCDF | 4xh2JKj | 4z5rUTI | 4kxzNME | |
| 4rrpGAM | 4xmnHLE | 4z5rWVE | 4kxzQPD | |
| 4rrpHBN | 4xnmBAD | 4z5rZYX | 4lkxABR | |
| 4rrpICO | 4xnmHLC | 4zffABC | 4lmqEIF | |
| 4rrpJDP | 4xnqBAD | 4zffHLD | 4lmqHLD | |
| 4rrpKEQ | 4xnqHLC | 4zfgHLA | 4m5zHLA | |
| 4rrpLER | 4xrcBAD | 4zs6CDB | 4m7lHLT | |
| 4tsaHLA | 4xrcHLC | 4zs6HLA | 4mwfABD | |
| 4tsbHLA | 4xtrCDB | 4zypDEC | 4mwfHLC | |
| 4tscHLA | 4xtrEFA | 4zypFGB | 4mxvFEB | |
| 4ttdCDB | 4xvjHLA | 4zypHIA | 4mxvHLA | |
| 4ttdHLA | 4xvuCDB | 4zypJLC | 4mxvYXD | |
| 4u6vHLA | 4xvuEFA | 4zypKMA | 4mxwHLA | |
| 4u6vKMB | 4xvuIJH | 4zypNOB | 4mxwWVX | |
| 4ut6HLA | 4xvuKLG | 5anmBAG | 4n0yHLA | |
| 4ut6IMB | 4xwgHLA | 5anmDCE | 4nhhIFD | |
| 4ut9HLA | 4xwoCDA | 5anmHLF | 4nhhMKB | |
| 4ut9IMB | 4xwoEFB | 5bo1HLB | 4nhhOQC | |
| 4ut9JND | 4xwoIJG | 5bo1IMA | 4nhhRPN | |
| 4ut9KOC | 4xwoKLH | 5bv7CBA | 4nnpHLA | |
| 4utaHLB | 4xwoOPM | 5bv7HLA | 4nnpXYB | |
| 4utaIMA | 4xwoQRN | 5bvpHLI | 4np4HLA | |
| 4utbHLA | 4xwoUVS | 5c6tHLA | 4np4IMA | |
| 4utbIMB | 4xwoWXT | 5c7xHLA | 4nztHLM | |

Antibodies with very close sequence homology (90% or greater) were excluded in advance using cd-hit. In this regard, only antibodies with sequence homology of less than 90% for both heavy chain and light chain were kept. For antibodies with an antibody structure comprising not only variable domains but also constant domains, those were also included.

The three-dimensional structure of each antibody is registered in PDB. The epitope can also be found from the structural data.

Furthermore, if only one antibody is deemed to recognize an identical epitope, the antibody was excluded.

The IDs of selected structures in PDB are the following. 1a2yBAC 1ahwBAC 1bvkBAC 1g7jBAC 1jpsHLT 1orsBAC 2a01DCA 2eizBAC 3d9aHLC 315wBAJ 315xHLA 4g6aCDB 4gagHLP 4hs6BAZ 4tsaHLA 4tscHLA 4y5vABC 4y5yABC. First, all antibodies were confirmed to be classified by the respective epitope (so-called answer for “checking answers”). This was performed by the following method using a three-dimensional crystal structure.

(1) Crystal structures of antigens were superimposed using the program RASH (see Rapid A S H, Daron M Standley, Hiroyuki Toh, Haruki Nakamura BMC Bioinformatics. 2007; 8: 116. Published online 2007 Apr. 4. doi: 10.1186/1471-2105-8-116). If the structural similarity score was higher than a threshold value, formula 1

$$\begin{matrix}[ Numeral 8 ] & \\ {S}_{kl} = {e}^{- {( \frac{{r}_{1} [ k ] - {r}_{2} [ l ]}{{d}_{0}} )}^{2}} , & ( 1 )\end{matrix}$$

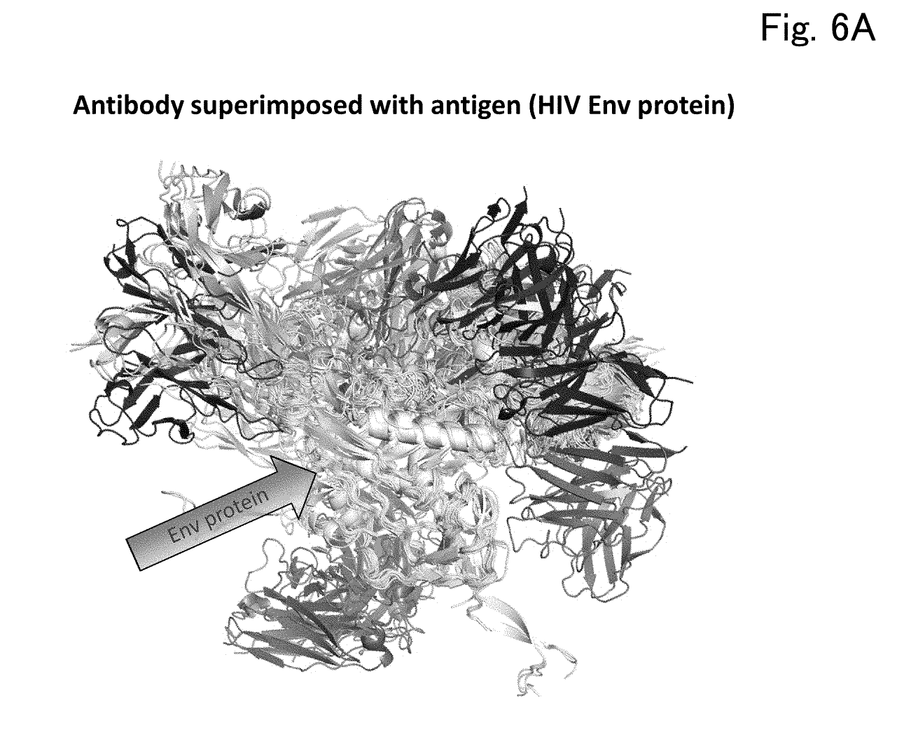

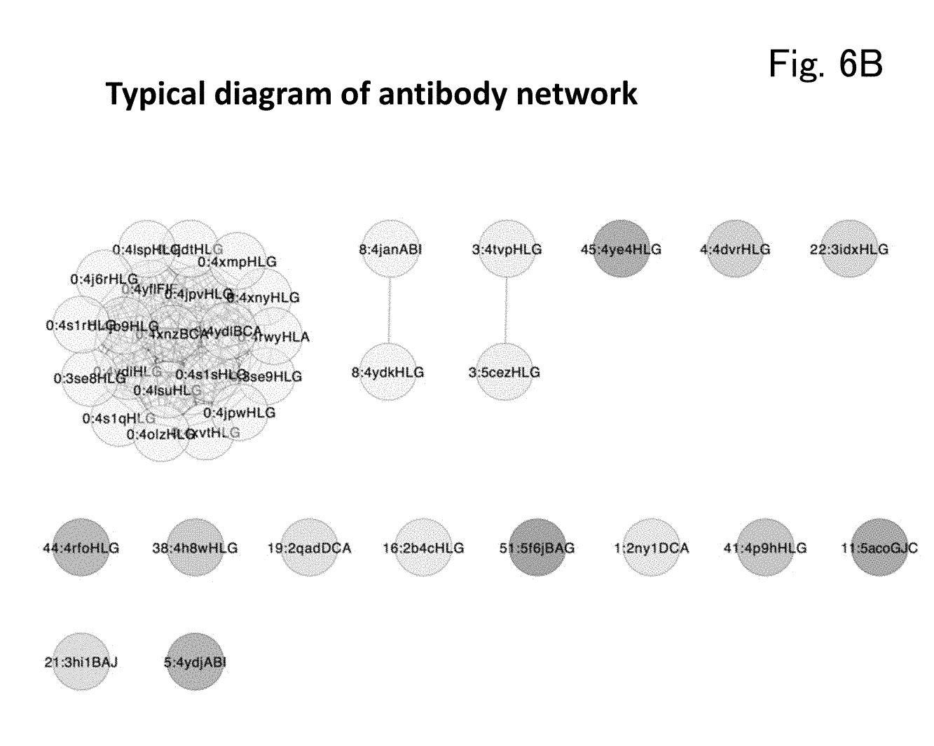

was used to evaluate the structural similarity of antibodies (when antigens are superimposed) (refers to the distance of each superimposed residues evaluated by formula (1) <Numeral 5>). Superimposed residues were added, which was divided by the RASH score of two superimposed antibodies, whereby an “epitope similarity score” was obtained (0-1). If the ASH score of the antigen was lower than a threshold value, the “epitope similarity score” was 0. This score was then used for generating a network of “true (=answer)” (FIG. 6).
(2) A structural model for all antibodies was produced. In this regard, a blacklist (sequence homology<85%) was used for structural modeling to avoid sequence homologous models. In this regard, an updated version of KOTAI Antibody Builder (Yamashita K, et al. Bioinformatics 30, 3279-3280 (2014)) was used.
(3) The following similarity features were calculated for all anti-HIV antibody pairs.

Aligned length in CDR1-3 for each of heavy chain and light chain

Difference in length in CDR1-3 for each of heavy chain and light chain

Ratio of NER to aligned length in CDR1-3 for each of heavy chain and light chain

Number of matching residues per aligned length in CDR1-3 for each of heavy chain and light chain

Aligned length of framework regions for each of heavy chain and light chain

Difference in length of framework regions for each of heavy chain and light chain

Ratio of NER to aligned length of framework regions for each of heavy chain and light chain

Number of matching residues per aligned length of framework regions for each of heavy chain and light chain

NER of framework regions for each of heavy chain and light chain

wherein NER is (Nearly equivalent residues) represented by [Numeral 7].
(4) The features were used for learning of support vector machine (SVM). SVM evaluated as follows using 5-fold cross validation. A machine learning library called scikit-learn was used. The kernel function was “linear”, and class_weigh option was “balanced”.
(A) All possible anti-HIV antibody pairs (for same or different epitopes) were separated randomly into a learning set and a verification set, where a sampling methodology called StratifiedKFold was used.
(B) SVM learned to distinguish anti-HIV antibodies recognizing the same epitope (positive) and those recognizing different epitopes (negative), and verified the performance using the verification set.
(C) (B) was repeated 5 times while changing the verification set.
(D) (A) to (C) were repeated 100 times while changing the random number for separating into a set.

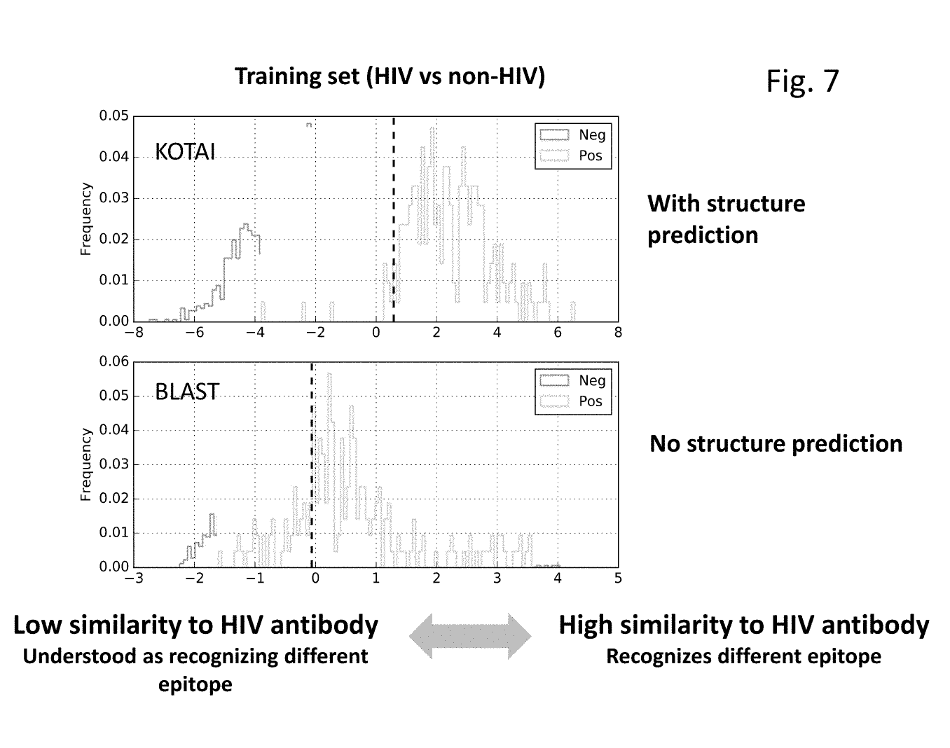

The results are shown in FIG. 7.

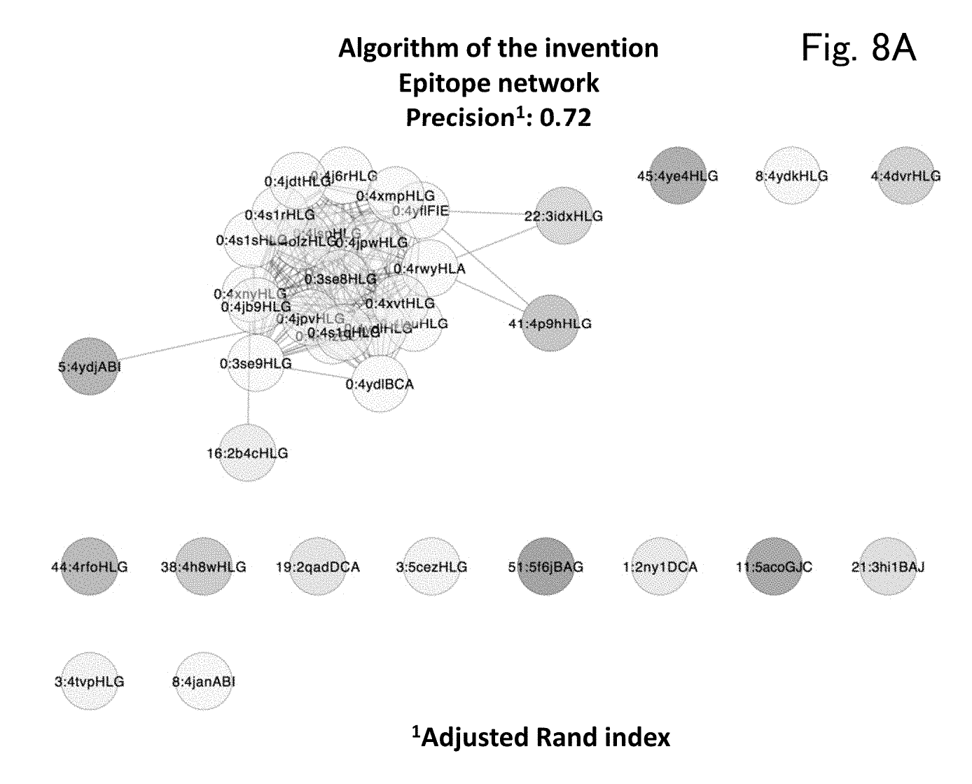

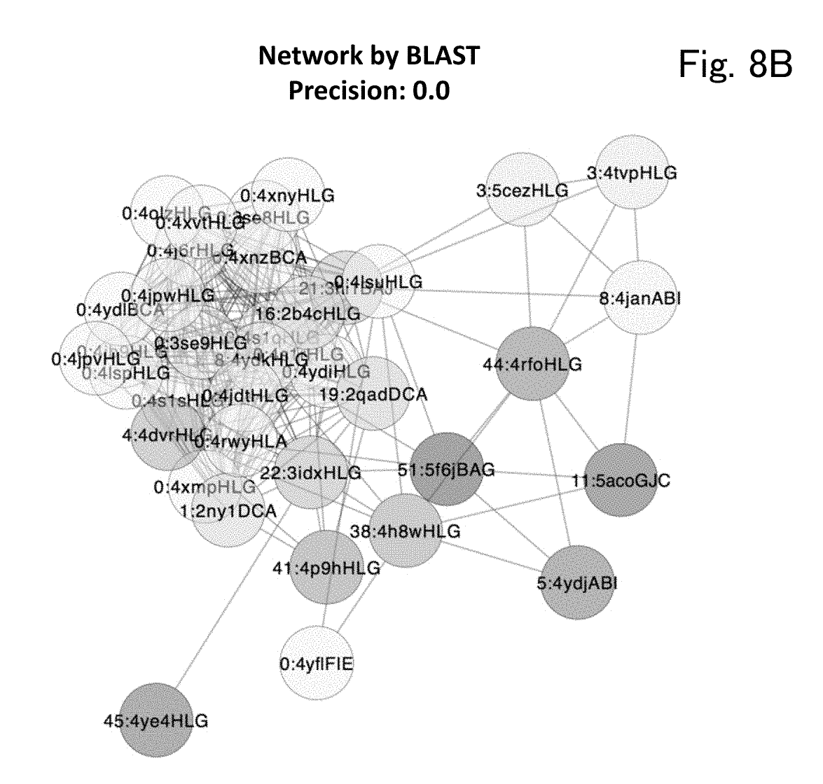

SVM was used to output a distance matrix for each pair. Finally, all of the anti-HIV antibodies were clustered using a distance matrix. The results are evaluated by the similarity to the true network. The results are shown in FIG. 8 with the network created by sequence similarity (similarity by alignment obtained with an existing software BLAST).

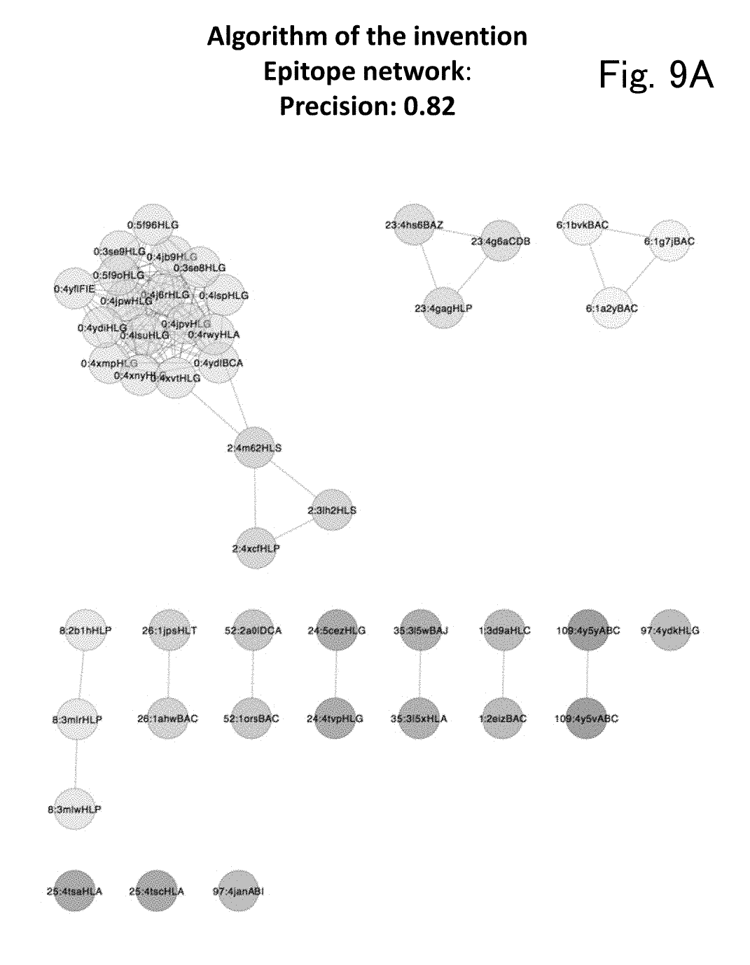

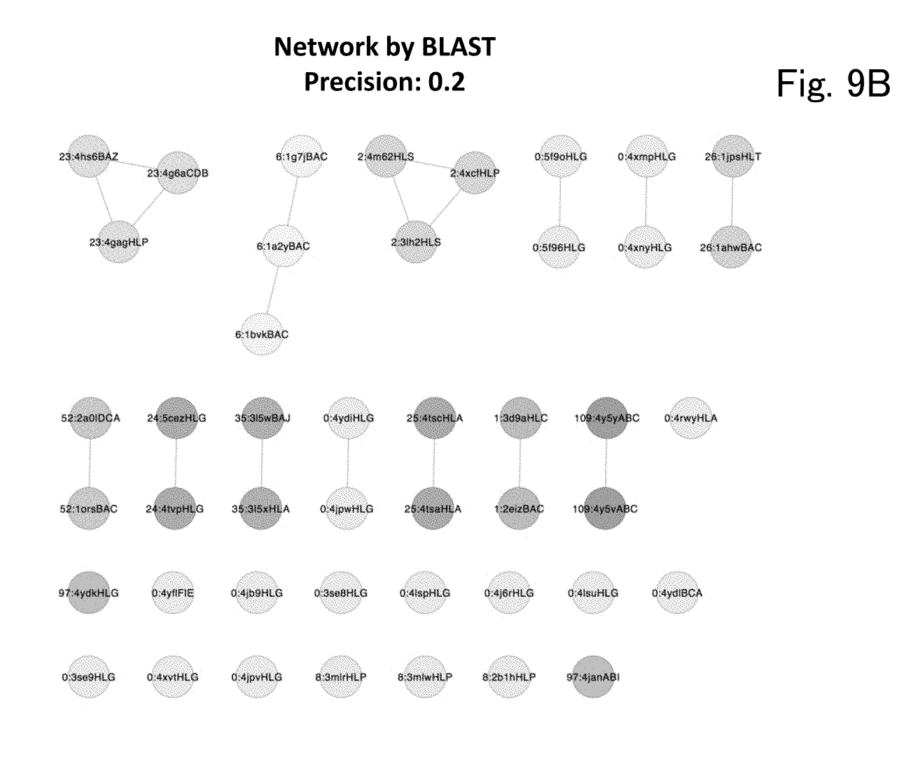

A set consolidating anti-HIV antibodies and non-anti-HIV antibodies was also clustered with a distance matrix obtained with SVM for anti-HIV and non-anti-HIV antibodies (FIG. 9). For clustering, group average method (average linkage clustering), which is one of the hierarchical clustering methodologies, was applied using the scipy module of Python. Those with a maximum distance of less than 0.85 were considered identical clusters.

The results in FIG. 8 clearly show that the proposed invention can identify antibodies with a common epitope better compared to inventions using only sequence similarity. For sequence similarity, all are in a single cluster, but the largest cluster is away from other epitopes in the present invention. This is quantified by an adjusted Rand index, which evaluates the similarity to the true cluster (FIG. 6). The present invention resulted in a Rand index of 0.72, while this is 0 for sequence similarity.

When anti-HIV antibodies and non-anti-HIV antibodies were consolidated, anti-HIV and non-anti-HIV d₀ not form a single cluster in the present invention, and the largest HIV cluster was again identified. Meanwhile, a large cluster could not be formed with sequence homology. The Rand indices were 0.82 and 0.2, respectively.

### Example 2: Example of Mapping NGS Data to Cluster Based on PDB Data Constructed in Example 1

This Example uses the cluster based on PDB database constructed in Example 1 to map NGS data and examine the prediction accuracy of the present invention.

The SVM constructed in Example 1 is applied without changing a parameter or the like to an antibody sequence (NGS antibody sequence) obtained by a single cell next generation sequencing (e.g., Tan et al., Clinical Immunology, 2014, 151, 55) of several 10s <61> B cells with an unknown antigen from peripheral blood obtained from HIV positive donors <each of the donors has passed the examination of the ethics committee established in accordance with the guidelines of the country or region where the sample was obtained (US or the like) or the international guidelines (ICH) and meet the guidelines of the Declaration of Helsinki or the like>. Application without any change indicates that consistent SVM can be applied or SVM created previously based only on existing data can be applied to new data, and indicates that SVM was created in Example 1 using data that is sufficient for classifying data of Example 2. The SVM created in Example 1 indicates that correct clustering can be performed on data for which the user does not known the answer. This is evidence demonstrating the effect of the invention.

It is examined whether the SVM using a known antigen-antibody structure constructed in Example 1 is also effective for an unknown sequence by the above operation. A structure model produced based on the NGS antibody sequence of this Example (using Kotai Antibody Builder) <used in Example 1; see Yamashita, K. et al. Bioinformatics 30, 3279-3280 (2014); parameters are the same as in Example 1> and The PDB structure considered in Example 1 (same as Example 1) are used to calculate the features of each of the sequences and structures that are the same in Example 1 and input the amount in SVM to create a distance matrix. The items and parameters used are the same as in Example 1. The same procedure described in FIGS. 6 to 9 is used.

RASH was used herein for superimposing framework regions. PDB structures were drawn in the same manner as Example 1, where a network is drawn so that each of the NGS antibodies is connected only to a PDB structure with the shortest distance. If a distance matrix is created in network construction, the condition of “connected only to a PDB structure with the shortest distance” is determined by finding the distance from all PDB structures in the distance matrix in the program used and selecting the shortest. As a result, all NGS antibodies were determined to have a distance that is the shortest to one of the PDB structures belonging to an HIV antibody cluster created in Example 1, i.e., determined as recognizing one HIV antibody epitope. In this regard, a connection was simply made to a base structure with the shortest distance. In fact, these newly obtained NGS antibody sequences were experimentally shown as anti-HIV antibodies, demonstrating the efficacy of the methodology of the invention.

### Example 3: Identification of Amplified Cluster after Vaccination

This Example identifies an amplified cluster after vaccination. Data described in Wiley et al., Science Trans. Med. 2011, 93, 1 is applied for the data thereof.

A host animal such as a BALB/c mouse (available from CHARLES RIVER LABORATORIES JAPAN, INC. and the like) is immunized with an antigen of a malaria parasite (Plasmodium vivax). Upon immunization with this antigen, the animal is immunized separately or concomitantly with various adjuvants (suitable amount of GLA-SE available from IDRI or R848-SE available from 3M Pharmaceuticals (e.g., 20 μg)). The mouse is immunized again on week 3 and week 6 after immunization by the same immunization procedure as the first immunization in accordance with a standard immunization procedure. A blood sample is obtained after 7 weeks from the first immunization. A blood sample is similarly obtained from a BALB/c mouse that has not been immunized.

These antibody heavy chain sequences are analyzed by the Long-read MPSS method <see Long-read Massive Parallel signature sequencing; Wiley et al., Science Trans. Med. 2011, 93, 1>. The repertoire of the immunized mouse (estimated to be about 5000 to 10000 sequences) and the repertoire of the BALB/c mouse that has not been immunized (estimated to be about 2000 to 4000 sequences) are compared (see Example 1 for creation and comparison of repertoires). The analyzed sequences are estimated to be about 10000 in total. A heavy chain and a light chain are generally required as inputs, but a three-dimensional model is produced with Kotai Antibody Builder (see Example 1 and the like) which enables the omission of calculating the light chain portion to produce a structural model of only a heavy chain. Of all the sequences, it is predicted that structure modeling was successful in about 70 to 80% of the sequences obtained from each of the unimmunized mouse and immunized mouse.

In accordance with the methodology proposed in the present invention, the framework regions of each of the structures are first superimposed using the RASH program, and then the structural similarity and sequences of each structure pair are evaluated. The SVM constructed for a structure of only the heavy chain is used herein. The method of SVM construction is as follows.

(1) SVM was trained using the PDB structure used in Example 1. In this Example, cd-hit is used to select only those with a degree of match in the heavy chain sequence of at least 90% thereamong. The superimposition methodology and the feature used are the same as in Example 1. However, information for light chains was not used. The specific value of match in sequences can be appropriately changed. About 85 to 90% can be employed as a good threshold value.
(2) Next, similarity to a known antibody structure (e.g., PDBID: 4k2uH, 4k4mH, 4qexH) for the antigen used in this Example is examined for sequences derived from each of unimmunized sample and immunized sample. As a result, it is estimated that structures judged to have about 3 to 5% of similarity (distance is <0.1) are found from each of the immunized sample and unimmunized sample (wherein structures found to be similar to a plurality of PDB structures are counted as such (a plurality of times).

As a result, the p value is estimated to be less than 0.05 (Chi-squared one-tailed test), and the immunized sample is shown to include significantly more structures that are similar to an antibody to a known antigen.

### Example 4. Clustering of Greater Size

In this Example, results of analysis on a larger data set (several 10s of thousand sequences) are shown. This Example uses data for humans inoculated with an antigen of a malaria parasite. Structure modeling for all sequences is performed with Kotai Antibody Builder in accordance with Example 1. In accordance with the methodology proposed in the present invention, the framework regions of each of the structures are first superimposed using the RASH program, and then the structure similarity of each structure pair is evaluated.

This Example does not consider sequences and evaluates only structural similarity.

$$[ Numeral 9 ]$$

$${D}_{geo}^{H , L} = \frac{{\sum }_{c ∈ \{ H , L \}} {\sum }_{i ∈ \{ 1 , 2 , 3 \}} {w}_{c} {len}_{c , i} {ner}_{c , i}}{{\sum }_{c ∈ \{ H , L \}} {\sum }_{i ∈ \{ 1 , 2 , 3 \}} {w}_{c} {len}_{c , i}}$$

wherein lenₖ is the aligned length, and nerₖ of a CDR region is a normalized Gaussian similarity score.

$$[ Numeral 10 ]$$

$$\frac{1}{{N}_{align}} {\sum }_{i}^{{N}_{align}} {e}^{- {( \frac{{r}_{i}^{q} - {r}_{i}^{t}}{4} )}^{2}}$$

Furthermore, 1 and 0.5 were each used as weight wₖ.

Next, the group average method (threshold value=0.1) is used to cluster all sequences.

Antibodies to about 20 vaccine constituent elements published in the IMGT database are selected to evaluate similarity to the structures contained in the data set. For structural similarity, the above formula is used, and similarity (=1−distance) of 0.9 or greater is considered similar. It is estimated that similarity with a known antibody is found in about 5 to 10% of the structures among several 10s of thousands of sequences.

Antibody pairs (100×100=about 10000) for which an antibody donor has identified an antigen are evaluated as to whether an antibody pair with a shorter distance targets an identical antigen. As a result, it is estimated that the correct pair of interest is found at a ratio of 20 to 30% among pairs with a distance of less than 0.1 and a ratio of 5 to 10% among pairs with a distance of 0.1 or greater. It is estimated that this is a statistically significant result (p≈10⁻⁶). This result meets the working hypothesis of antibodies with a shorter structural distance recognizing an identical epitope proposed by the inventors. Since epitopes that are very similar in terms of the sequence and structure cannot be distinguished in principle, an aggregate of similar antigens that can be structurally categorized in the same category can be determined to be identical.

### Example 5. Clustering of Cytomegalovirus Specific CD8+ T Cell Receptors

In this Example, cytomegalovirus specific CD8+ T cell receptors were clustered.

Cytomegalovirus (CMV) is a cause of a severe disease for patients with no immunocompetence, e.g., patients who have undergone organ transplantation. For this reason, development of a vaccine for CMV is needed. When infected with a CMV virus, CMV specific CD8⁺ T cells are produced. Many sequences of CMV specific CD8⁺ T cells have been identified. Since a CMV sequence presented by HLA varies by the HLA type, the T cell repertoire produced by each donor is dependent on the HLA type. Therefore, a method for monitoring the efficacy of a vaccine includes examining the amount of production of CMV specific TCRs after vaccination.

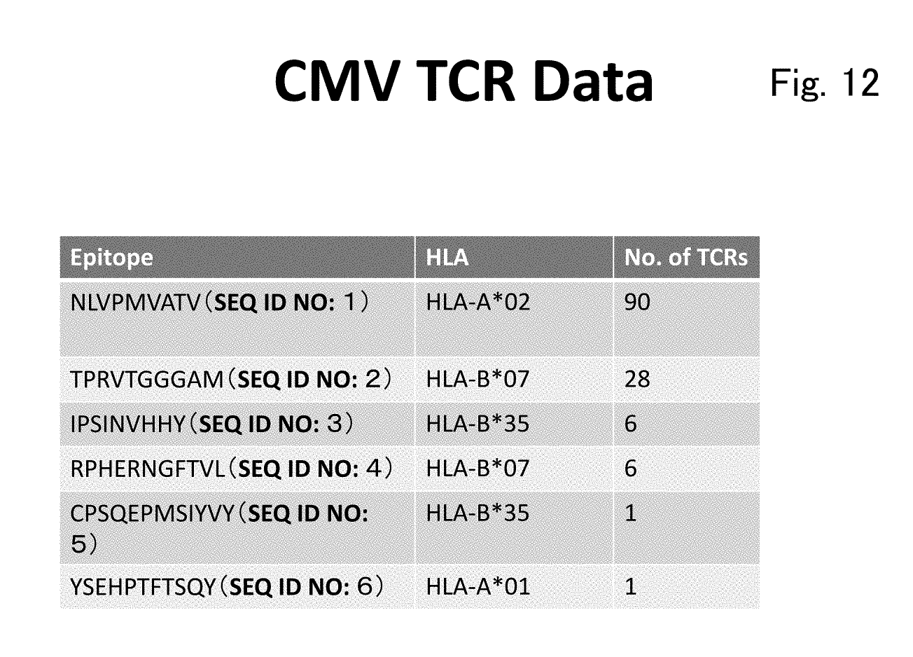

FIG. 12 shows epitope sequences (SEQ ID NOs: 1 to 6) (based on the following articles in Table 3).

TABLE 3

| | |
|---|---|
| Arakaki, et al., Biotech. Bioeng. 2010, 106, | Babel, et al., Am. J. Transplant., 2012, 12, |
| 311 | 669 |
| Bockel et al., J. Immunol., 2010, 186, 359 | Brennan, at al., J. Virol. 2007, 81, 7269 |
| Brennan, et al., J. Immunol., 2012, 188, | Day, et al., J. Immunol., 2007, 179, 3203 |
| 2742 | |
| Dziubianau, et al., Am. J. Transplant., | Giest, et al., Immunol., 2012, 135, 27. |
| 2013, 13, 2842 | |
| Hamel, et al., Euro. J. Immunol., 2003, 33, | Janbazian, et al., J. Immunol. 2012, 188. |
| 760. | 1156 |
| Klarenbeek, et al., PLoS Pathog., 2012, 8, | Khan, et al., J. Immunol., 2002, 169, 1984 |
| e1002889 | |
| Khan, at al., J. Infect. Dis., 2002, 185, | Klinger, et al., PLoS ONE, 2013, 8, e74231 |
| 1025 | |
| Koning, et al., J. Immunol. Method., 2014, | Miconnet et al., J. Immnol., 2011, 186, |
| 405, 199 | 7039 |
| Nakasone, et al., Bone Marrow | Nguyen, et al., J. Immunol. 2014, 192, |
| Transplant., 2014, 49, 87 | 5039 |
| Peggs, at al., Blood, 2002, 99, 213. | Price, et al., JEM, 2005, 202, 1349 |
| Retiere, et al., J. Virol., 2000, 74, 3948 | Scheinberg, et al., Blood, 2009, 114, 5071 |
| Schub, et al., J. Immnol., 2009, 183, 6819 | Schwele, et al., Am. J. Trasnplant., 2012, |
| | 12, 669 |
| Trautmann, et. al., J. Immnol., 2005, 175, | Venturi, et al., J. Immunol. 2008, 181, |
| 6123 | 7853 |
| Weekes, et al., J. Virol. 1999, 73, 2099 | Weekes, et al., J. Immunol. 2004, 173, |
| | 5843 |
| Wynn, et al., Blood, 2008, 111, 4283 | |

from which HLA types binding to an epitope of CMV collected therefrom and TCR β chain sequences recognizing them (excluding those with a sequence match of 95% or greater by the cd-hit program) are derived.

TCR structures were modeled. The procedure of modeling is the following.

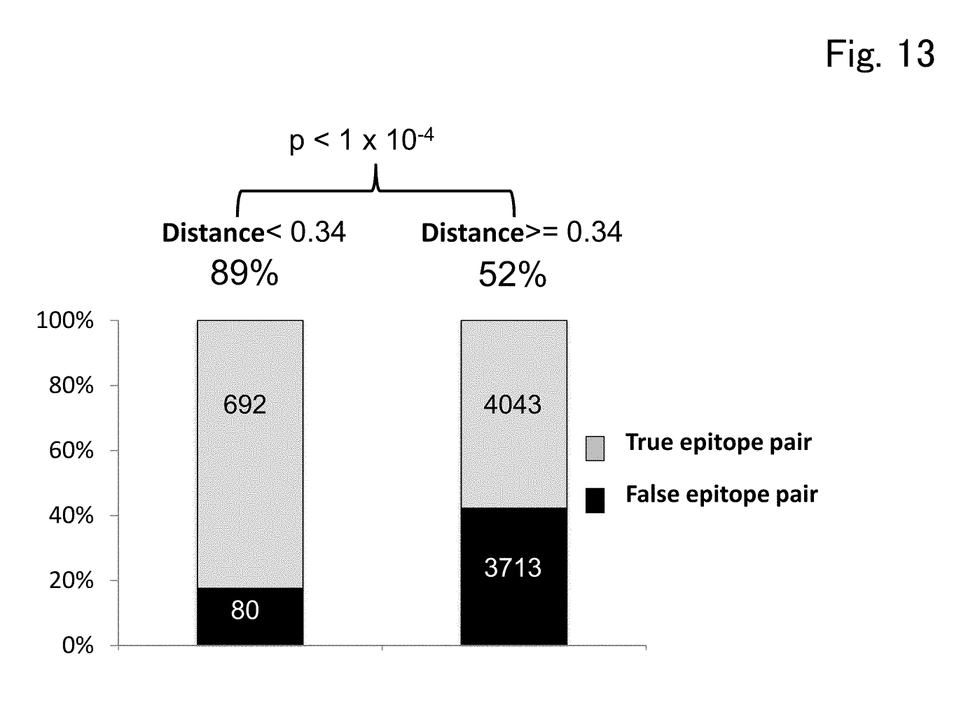

First, in accordance with the definition of IMGT, CDR3 regions were masked to search for similar PDB sequences to PDB with BLASTp. As a template of regions other than the CDR3 region, those with the smallest e-value were used. Default parameters were used. Furthermore, three structures of CDR3 regions were produced with spanner (Lis M, et al., Immunome Res. 2011, 7, 1). Oscar-star (Liang S, et al., Bioinformatics, 2011, 27, 2913) was used for side chain modeling. Furthermore, oscar-loop (Liang, S., J. Chem. Theory Comput. 2012, 8, 1820) was used for energy minimization and scoring of a CDR3 region to employ an energy minimum model. This resulted in successful structure modeling of 132 TCR β chain sequences. First, a stable region in a TCR structure was defined as a framework region by the same procedure in Example 1, and structures were superimposed using RASH, in accordance with the methodology proposed in the present invention. A distance matrix was created using SVM using a sequence characteristic and structure characteristic based on the superimposed structure, and clustering was performed. A machine learning library called scikit-learn was used herein for SVM. The kernel function was “rbf”, and class_weigh option was “balanced”. The threshold value was 0.34. TCR pairs were separated into two classes (pair distance is <0.34 and >=0.34) to evaluate whether the TCR pairs belonging to each class recognize an identical epitope (FIG. 13).

The result demonstrated that pairs with a shorter distance (group belonging to <0.34) had more pairs recognizing an identical epitope.

### Example 6 B Cell Screening (1)

This Example presents an example of applying this methodology of B cell screening.

A technology using the clustering of the invention can be applied to screening of B cells. Several applications are contemplated for screening of B cell repertoire. One method is to find an antigen of an antibody of interest from the antibody sequence, and another is a method of finding one that was unknown from an antibody sequence group of interest.

Examples of the first method include an example used in evaluating whether an experiment has been correctly conducted. Since a plurality of samples are sequenced at once in next-generation sequencing, there is generally a possibility of contamination. While it is difficult to analyze whether there is contamination, an antibody that recognizes an unintended antigen can be found to evaluate an experiment by screening using epitope clustering.

If an antibody that recognizes an unintended antigen is found at this time, this can be determined as contamination. Alternatively, the hypothesis can be revised.

More specifically, if, for example, an antigen of a cluster (or, for example, up to the 10th cluster as a ranking) accounting for 1% or more of the entire sequence count is identified and the antigen is unrelated to the vaccine, contamination can be suspected.

Similarly for vaccine purification, an antigen (adjuvant) is readily envisioned for antibody production with respect to an unintended adjuvant or the like, so that immunogenicity can first be used concomitantly with detection with co-immunoprecipitation or the like with for example serum. The method of the invention can provide information that cannot be obtained with co-immunoprecipitation in terms of being able to identify unintended contaminant.

In evaluating vaccines, the quality of vaccine purification, whether an antibody is produced with respect to, for example, an unintended adjuvant, or the like can also be evaluated.

In Japan, influenza vaccines are generally produced using chicken eggs. Thus, there is a possibility of residual egg components, i.e., egg white or lysozyme, upon vaccine purification. For example, increased antibody titer to components of eggs is expected with poor vaccine purification.

In such a case, similarity to a known antibody is evaluated for the B cell repertoire of mice inoculated with an influenza vaccine. Blood of mice is collected after 1 week from vaccination. For known antibodies, structure data and sequence data with a known antigen registered in a public data base are used. For sequence data, a structural model is produced. Similarity between each known antibody and an antibody in the repertoire is evaluated in accordance with Example 1 by the methodology of the invention. If a plurality of known antibodies are selected within the threshold value for determining an antibody to be similar, the most similar antibody is selected. Clusters are created around each known antibody by the above method described in Example 1 or the like, and especially large clusters are examined as to whether an unintended antibody such as an anti-lysozyme antibody, anti-adjuvant antibody, or anything completely unrelated is contained to evaluate whether an experiment has an intended result.

<Example of the Whole>

There are cases where it is desirable to identify an antibody group of interest and to select those with high binding capability or neutralization capability. In such cases, the methodology proposed can be used to more readily and efficiently select an antibody of interest. The methodology will be discussed.

It is assumed that B cell receptors (BCR) of interest have been already identified (e.g., by FACS and neutralization capability IC₅₀ with respect to a plurality of viral strains) as broad neutralizing antibodies of HIV. PBMC is produced from peripheral blood of a donor comprising a BCR of interest, and a plasmablast B cell of interest is selected by FACS to perform single cell sequencing. If there are several 10s of thousands of sequences and it is desirable to examine another antibody (e.g., find an antibody with higher affinity to a specific viral strain or the like), but is unclear which should be preferentially examined, structural models are produced and superimposed to obtain features of structural and sequence similarity in accordance with Example 1. This is inputted into SVM to create a structure cluster. At the same time, for example IgBLAST (Ye, et al., NAR, 2013, 41, W34) or IMGT HighV/QUEST (Brochet et al., NAR, 2008, 36, W503) is used to assign a V(D)J gene to each sequence, which is divided by sequence line (lineage or clone) depending on the gene and CDR3 sequence used. Various forms of the method have been proposed and are known in the art (e.g., DeKosky, et al., Nat Biotechnol. 2013, 31, 166).

While different methods yield different division results, the difference is minor, such that this would not be an issue for the purpose of the invention. Next, it is examined which structural cluster the identified BCR of interest belongs. If it is desirable to examine the antibody of interest and the like relatively broadly, not only the structure cluster to which the antibody belongs, but also all sequence lines belonging to the structure cluster are compared. In other words, all sequence lines belonging to the same structure cluster as the BCR of interest can be examined by combining with sequence analysis. Since the methodology proposed in the present invention performs clustering with an epitope, not only the sequence line to which the BCR of interest belongs, but functionally very similar broad lines can be efficiently analyzed. If it is desirable to narrow/broaden the BCR sequences to be examined, efficient search and evaluation are enabled by changing the threshold value for structure clustering to further divide/consolidate clusters, or further dividing/consolidating sequence lines by common somatic hyper mutation with sequence analysis and selectively choosing BCRs that are far apart or close to the identified BCR.

### Example 7 B Cell Screening (2)

This Example describes an example of the second method for B cell screening.

An effective influenza vaccine induces B cells producing an antibody that neutralizes broader viral strains at once. An attempt to develop a vaccine using a stem region of genetically well conserved influenza surface protein (hemagglutinin) as a target epitope is ongoing. It is important in evaluation of this vaccine to distinguish an antibody binding to a stem region from other antibodies. Several antibody groups that recognize a stem region are already known. A characteristic sequence motif thereof has been reported (e.g., Gordon Joyce et al., 2016, Cell 166, 609). Evaluation of a vaccine requires that antibodies recognizing a target epitope are comprehensively sorted out, but there is no guarantee that an existing sequence motif comprehensively includes antibodies recognizing a target region.

In this Example, type A influenza hemagglutinin (HA) is separated into Group 1 and Group 2. A human is immunized with an H1 protein belonging to Group 1, and blood is collected after a week. FACS is used to select B cells binding to HA belonging to Group 1 and Group 2, and the sequences thereof are obtained by next generation sequencing. These are clustered using the methodology proposed in the present invention in accordance with the methodology of Example 1 or the like based on a known influenza antibody sequence. This enables separation into a cluster comprising sequences similar to a known antibody sequence and a cluster comprising an unknown antibody sequence. For the cluster comprising sequences similar to a known sequence, it is examined whether a sequence motif that has been reported can sufficiently cover the cluster. The presence of a sequence that does not correspond thereto means that the sequence motif is not sufficient. Ideally, it is experimentally examined whether an identical epitope as the known one is recognized. For this purpose, a crystal structure analysis or the like can be conducted. Crystal structure analysis can be similarly conducted for the unknown cluster for experimental confirmation.

### Example 8: aPAP (Disease Specific Marker)

This Example describes an example of a methodology to identify a disease specific marker.

As an example thereof, autoimmune pulmonary alveolar proteinosis (aPAP) is used.

Autoimmune pulmonary alveolar proteinosis (aPAP) is a rare respiratory disease (0.37 patients per 100000 persons) wherein a surfactant-like substance builds up in the alveolar space, resulting in dyspnea. Patients thereof are known to have an anti-GM-CSF antibody. In addition, there is a report of, for example, pathological reproduction of GM-CSF knockout mice (G Dranoff, et al., Science 1994, 264, 713-716) and the like, suggesting pathogenicity of an anti-GM-CSF antibody. Recently, it is known that autoantibodies recognizing multiple different epitopes of GM-CSF neutralize GM-CSF in vitro and decompose an immune complex comprising GM-CSF in vivo (Piccoli, et al., Nature Communications 2015, 6, 7375). In this regard, a cluster of auto-BCRs recognizing different epitopes is identified using B cells obtained from the peripheral blood of a patient, which are compared with the severity of the patient.

While it may be possible to find a cluster from a B cell repertoire and compare them with severity, the antigen is already known for this disease, so that it is easier to select a B cell with an anti-GM-CSF from the peripheral blood by FACS and obtain a plurality of sequences by the Sanger method to find a cluster comprising them from the B cell repertoire. Ideally, the competitiveness of the resulting anti-GM-CSF BCRs is analyzed by an in vitro experiment (e.g., Biacore) and/or the clustering methodology proposed in the present invention is used in accordance with Example 1 to divide the resulting anti-GM-CSF BCR by epitopes.

A B cell repertoire of each patient is obtained by immune cell sequencing technology from peripheral blood patients with a plurality of different severities. Furthermore, a similar BCR sequence is selected with the clustering technology proposed in the present invention in accordance with Example 1 based on a “representative” anti-GM-CSF BCR sequence. A BCR sequence detected by FACS is not necessarily found in a repertoire obtained by a next generation sequencer and vice versa. Thus, it is sufficiently possible that it is important for expressing severity in clusters with an unknown antigen. For evaluation of the association with the above severity, a repertoire excluding known anti-GM-CSF BCR antibody sequences is clustered with the methodology proposed in the present invention in accordance with Example 1, and a characteristic cluster in patients with high severity, or a cluster with high correlation between severity and cluster size is selected.

In this regard, several patterns can be expected in selecting a marker that is the most correlated with severity.

1. N (e.g., 3) or more anti-GM-CSF BCR clusters are found. 1b. In addition to 1, the clusters account for (for example) 1% or more of the entire repertoire.
2. There is a cluster that is most correlated with severity, and a plurality (2 or more) of other clusters are found.
2b. A cluster that is important in terms of the quantitative relationship thereof is the largest, the respective size is nearly constant, and the like.

The present invention can be applied for identification of a disease specific marker by the following procedure.

### Example 9: Examination with B Cell Receptor (BCR)

This Example examined whether the clustering technology of the invention is suitable using B cell receptors (BCR). In this regard, the central hypothesis of the inventors is that BCRs having a similar sequence and structural characteristic have greater possibility of targeting an identical antigen and epitope than BCRs with different characteristics.

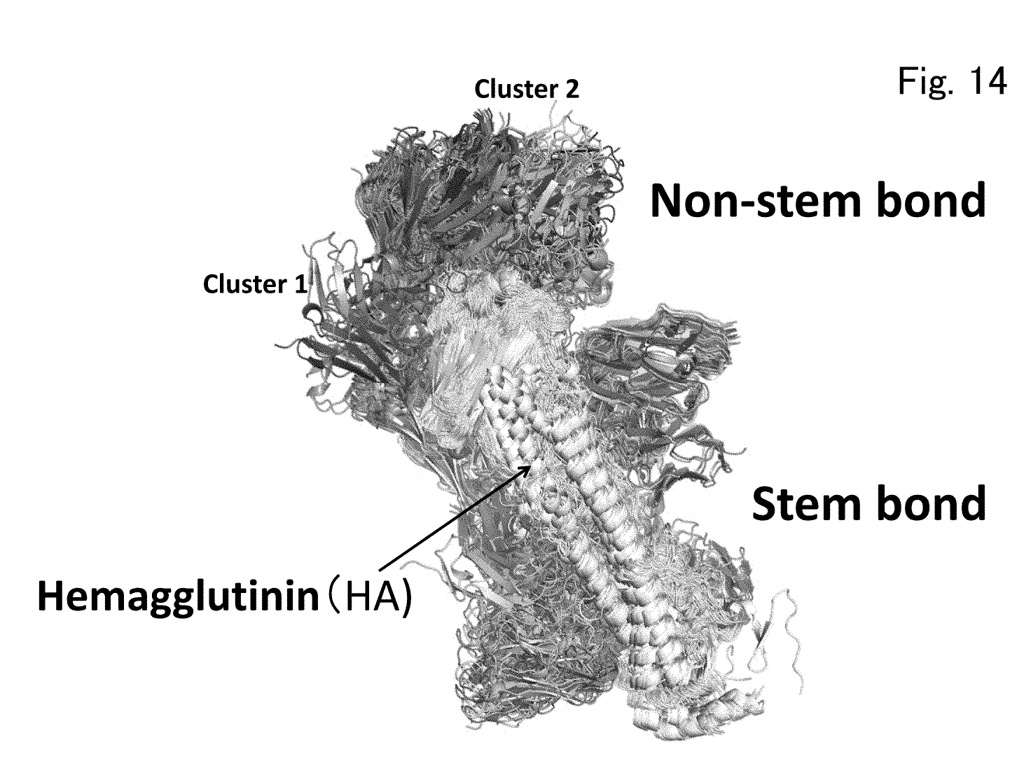

To test this hypothesis, the inventors used influenza hemagglutinin (HA) as a model antigen. HA can be roughly separated into two regions: stem and non-stem (FIG. 14). Each region consists of a plurality of epitopes. Since a stem epitope generally has a sequence and structure that are well conserved among various strains, a stem epitope has expectation as an epitope of a neutralizing antibody. HA is an axis symmetric trimer. The figure was created so that all BCRs are arranged on a common reference frame (i.e., so that BCRs occupy the smaller area (in the background of the figure) and two of the HA chains are exposed in the front as if HA is not bound; these “exposed” HA chains are actually similarly covered in BCRs). A non-stem binder submitted to the protein data bank (PDB) occupies about two clusters (labeled cluster 1 and cluster 2).

The methodology of this Example is described below.

(Materials and Methods)

(Characterizing of Antibody and BCR-Seq of Antigen Specific B Cells)

A highly efficient system of method was used, which enables combined analysis of Ig affinity profiling and immunoglobulin (Ig) gene repertoire from a single B cell sample developed by Professor Kurosaki of Osaka University.

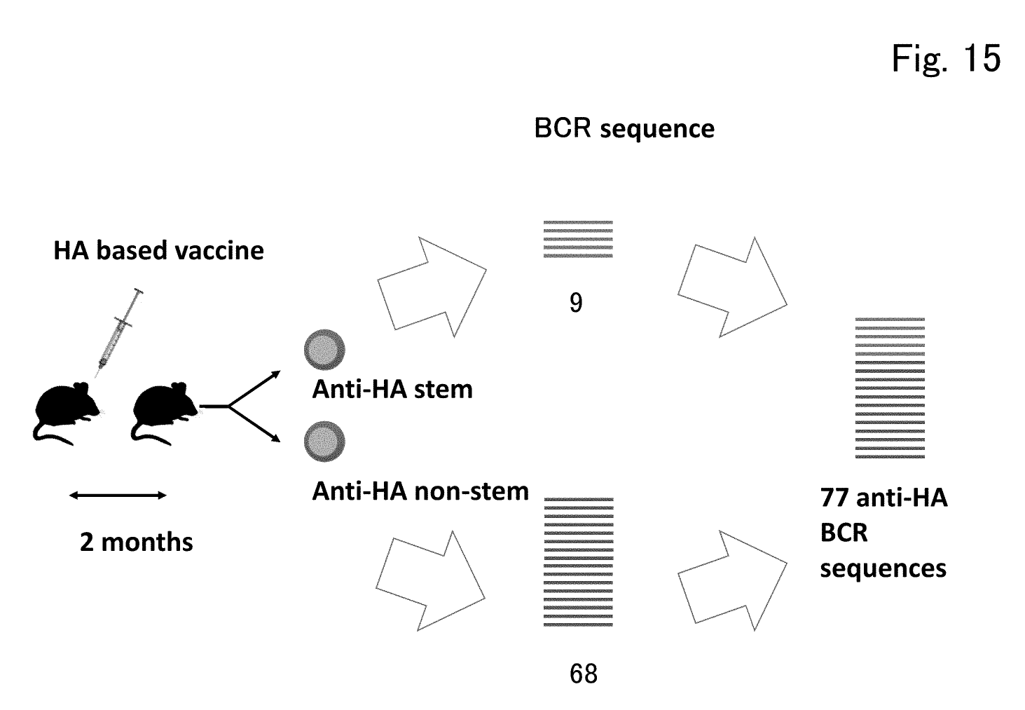

An experiment was designed to prepare a mouse to induce anti-stem BCRs and anti-non-stem BCRs (FIG. 15). First, a mouse was vaccinated with influenza hemagglutinin (HA). Flow cytometry was used to sort single cells for antigen (HA) specific germinal center (GC) or memory B cells from the vaccinated mouse. For each cell, Ig heavy chain and light chain gene transcripts were independently amplified by PCR, sequenced, and cloned into a mammalian expression vector.

Recombinant antibodies were produced in mammalian Expi293F cells to measure affinity to an HA antigen based on ELISA.

By using this method, the inventors associated Ig sequence information with antibody reactivity, and diversity in affinity and Ig repertoire was analyzed between immune tissues (e.g., spleen vs. lymph node), points in time (e.g., 2 weeks vs. 4 weeks after infection), and individual mice. The data was useful for understanding the mechanism of BCR clone selection and affinity maturation in immune responses to viral antigens.

9 stem binding anti-HA B cells and 68 non-stem binding anti-HA B cells were obtained by the above procedure.

(3D Modeling and Clustering)

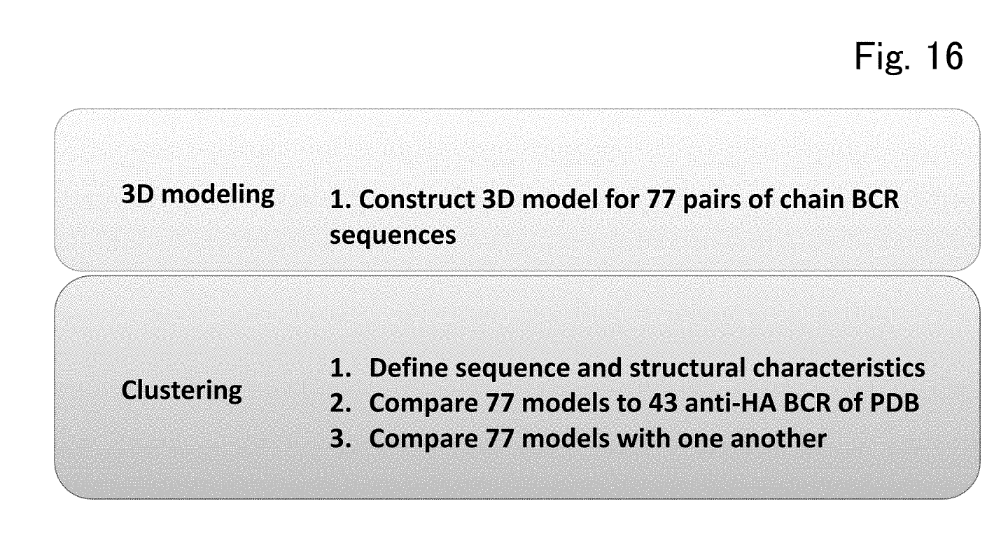

Sequence data were analyzed in two phases: 3D modeling and clustering (FIG. 16). The inventors performed the steps of the 3D modeling phase based on Kotai Antibody Builder as described in Example 1, other than using the method for selecting a template described below (BCR 3D modeling). In the clustering phase, the inventors first defined the sequence and structural characteristic, and then used these characteristics to compare 77 models to 43 known anti-HA BCRs obtained from PDB, and compared the 77 models with one another.

(BCR 3D Modeling)

A non-overlapping set of template variable fragment (Fv) sequences from humans, mice, and rats was used for multiple alignments using the restriction originating from the structural alignment in pairs described previously (Katoh, K. and Standley, D. M. MAFFT multiple sequence alignment software version 7: improvements in performance and usability. Mol Biol Evol 2013; 30(4): 772-780.) The inventors included sequences of a comprehensive set for framework templates. For CDR templates, the inventors prepared separate subsets for each length of each CDR in each chain type (BCR_L1-3, BCR_H1-3, TCR_A1-3, TCR_B1-3). A gap was no observed in 4 residues immediately upstream or immediately downstream of CDR and a column corresponding to the CDR of interest. In view of MSA m(i, j) (herein, i is an aligned sequence (row), and j is the aligned position (column)), the inventors defined sequence similarity between any pair of templates as the following formula:

Sᵢⱼ=Σₖw(k)B(m(i,k),m(j,k)) [Numeral 11]

wherein w(k) is a weighting vector, B(i, j) is a matrix of BLOSUM62 scores comprising an additional dimension as a gap penalty. The weighting w(k) is an adjustable parameter adapted to achieve the optimal result between Sᵢⱼ and structural similarity of sequence i and j for each CDR with a given length. In other words, the inventors used Monte Carlo and gradient descent path executed in the Theano python library to minimize the difference between S-based ranking and similarity based ranking.

The inventors can efficiently align a query sequence q, whose structure is desired to be predicted, to m without changing the alignment between templates (Katoh, K. and Standley, D. M. MAFFT multiple sequence alignment software version 7: improvements in performance and usability. Mol Biol Evol 2013; 30(4): 772-780.) To express a model of a given query, the inventors first estimated the length of CDRs by alignment to a framework MSA. Template (e.g., BCR_L-H or TCR_A-B) naturally forming a pair with the highest overall framework score are selected and used to define the directionality of two framework templates. Next, the inventors aligned a full-length query sequence to a suitable MSA for each CDR. The basis for using a full-length sequence in CDR MSA is that residues outside of a CDR can contribute to the stability thereof. RMSD superimposition of 4 residues in the front and back of a CDR was used as an anchor, and a CDR template with the highest score was transplanted into a framework template with the highest score. In each step, mismatch was monitored. If a mismatch is beyond a threshold value, the template with the highest score was replaced with a non-optimal template. A side chain that differs between query and template was reconstructed using a conformation frequently observed in a corresponding MSA column.

(BCR Model Clustering)

The inventors examined three CDR characteristics for clustering:

(a) structural similarity;
(b) sequence similarity; and
(c) difference in lengths.

Structural similarity for a given CDR was defined as described previously regarding protein structure alignment (Standley, D. M., Toh, H. and Nakamura, H. Detecting local structural similarity in proteins by maximizing number of equivalent residues. Proteins 2004; 57(2): 381-391.)

$$[ Numeral 12 ]$$

$$StrucSim = \frac{1}{N} {\sum }_{i}^{N} {e}^{- {( \frac{{d}_{i}}{{d}_{0}} )}^{2}}$$

wherein dᵢ is a distance between C-alpha atoms in residues aligned in two models, N is the length of alignment, and d₀ is a constant reference distance. For each model, structural similarity was defined as an average for 6 CDRs.

Sequence similarity for a given CDR was defined from the viewpoint of components of a BLOSUM 62 matrix of aligned residues. If an aligned residue pair consists of amino acids a₁ and a₂ for models 1 and 2, the inventors indicated the component of a BLOSUM62 a₁-a₂ matrix as Bᵢ, while the components of elements a₁-a₁ and a₂-a₂ on a diagonal line are indicated as Cᵢ and Dᵢ. The score for a given CDR was defined as follows.

$$[ Numeral 13 ]$$

$$SeqSim = {\sum }_{i}^{N} \frac{{B}_{i}}{MAX ( {C}_{i} , {D}_{i} )}$$

The difference in lengths was simply defined as the maximum difference in the lengths of CDRs for all 6 CDRs. This formula was used based on the knowledge that dividing by length or averaging CDRs are considered as having hardly any effect because different epitopes targeted by BCR often differ in terms of the length of CDR in a CDR.

Next, when the values were within the cutoff, clustering was performed by linking nodes.

(Determination of Character Threshold Value)

First, all PDB entries with two or more BCRs having different amino acid sequences targeting an identical epitope were clustered. As a result, 399 BCRs targeting 60 epitopes were obtained.

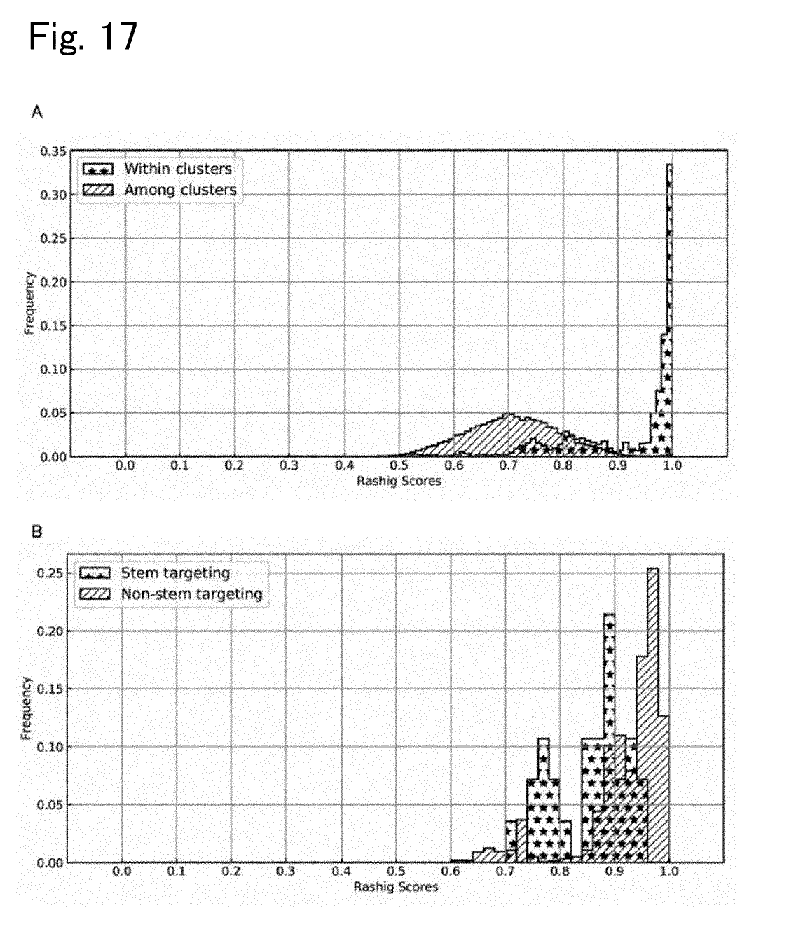

Next, the inventors calculated the StrucSim score within all BCRs and among all BCRs. As can be shown in FIG. 17A, most of intra-epitope pairs (e.g., within an identical epitope group) can be separated from inter-epitope pair (i.e., among different epitope groups) with a threshold value of about 0.9. Next, the inventors calculated the same StrucSim score for stem and non-stem mouse BCR models (FIG. 17B). In this regard, “stem” and “non-stem” classes were not completely separated due to the fact that they represent many different epitopes.

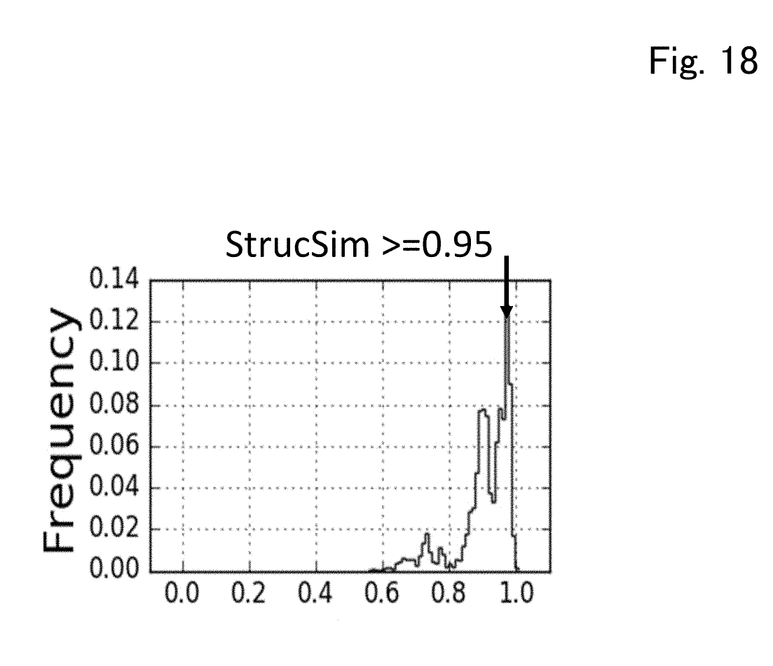

In this regard, the inventions set the threshold value of StrucSim to 0.95 to separate stem and non-stem into different epitopes (FIG. 18).

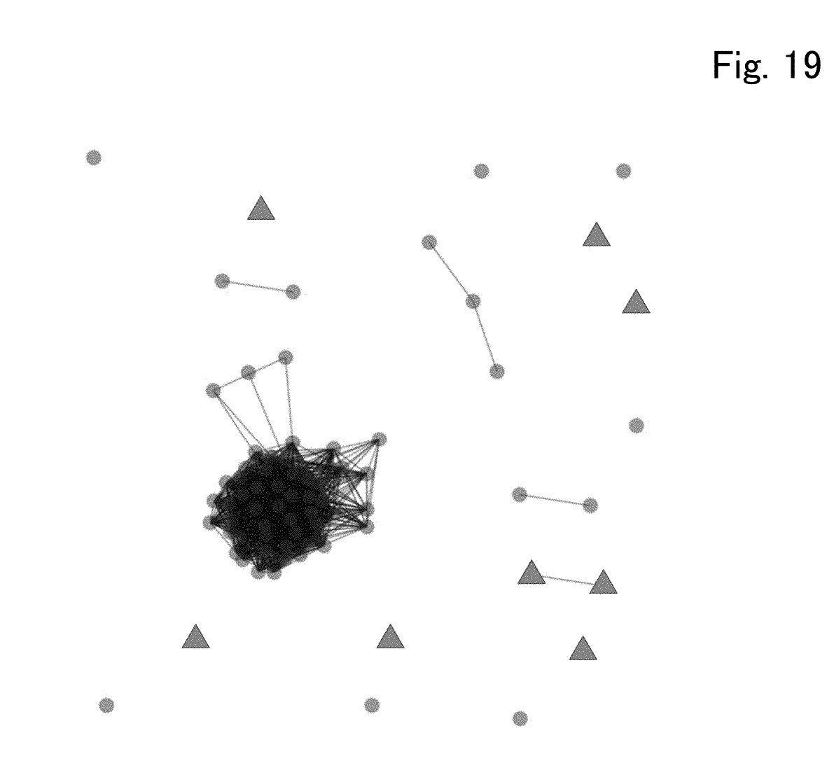

Clusters were made visible by using Python NetworkX graphviz package which draws a single line between paired portions with a matching characteristic within the threshold value (FIG. 19).

(Discussion)

When the inventors compared the models with one another, a high degree of similarity was found (FIG. 19). In particular, the majority of anti-non-stem BCRs formed a large cluster, which contained no anti-stem BCRs. In line therewith, two of the anti-stem BCRs clustered together. With analysis of known anti-stem BCRs, this class was confirmed to represent various epitopes and BCRs (see “Determination of character threshold value”). For this reason, lower clustering among anti-stem BCRs matches the experimental data.

It is important in this Example that non-stem and stem were able to be classified using experimentally confirmed BCRs, i.e., those assigned as non-stem and those assigned as stem were separated, which demonstrate the usefulness of the present invention. It is understood that further classification is possible by appropriately adjusting the threshold value.

A stem region not being separated can be explained in terms of a problem in data layer accumulated in PDB and biological meaning of a stem region. This is very consistent with the theories in the present invention. Specifically, the stem region and non-stem (also called Head or Stalk) region of influenza hemagglutinin (HA) are each large proteins with a large number of epitopes. It is known that the structures in PDB are mostly stem regions and non-stem regions recognizing a receptor binding site of sialic acid, which have drawn attention as a neutralizing antibody. Furthermore, it is known that a receptor binding site of a non-stem region is better conserved than those of a stem region (otherwise could not bind). Therefore, many antibodies appear superimposed in FIG. 14 (Cluster 2). Meanwhile, the stem region appears spread out because various strains (lines) are overwritten in FIG. 14 so that those neutralizing over several strains (line) do not necessarily neutralize all strains (different spectral bandwidths). In fact, strain (line) specific immunodominant sites (epitope) where a non-neutralizing antibody binds are known in a non-stem region (about 4 to 5 each). However, due to the low scientific interest, PDB database is considered to have accumulated a low number of crystal structures, which aptly clarifies the characteristic of data accumulated by the technology of the invention.

(Note)

As disclosed above, the present invention has been exemplified by the use of its preferred embodiments. However, it is understood that the scope of the present invention should be interpreted based solely on the Claims. It is also understood that any patent, any patent application, and any other references cited herein should be incorporated herein by reference in the same manner as the contents are specifically described herein. The present application claims priority to Japanese Patent Application No. 2016-181250 filed on Sep. 16, 2017 in Japan. The entire content thereof is incorporated herein by reference.

### INDUSTRIAL APPLICABILITY

Clinical application with high accuracy is possible for immune related diseases.

### SEQUENCE LISTING FREE TEXT

SEQ ID NOs: 1 to 6: Epitope sequences used in Example 5.

1. A method for classifying whether a first immunological entity and a second immunological entity are identical or different for an epitope to be bound thereby, the method comprising the steps of:
(1) identifying conserved regions of amino acid sequences of the first immunological entity and the second immunological entity;
(2) producing three-dimensional structure models of the first immunological entity and the second immunological entity;
(3) superimposing the conserved regions of the first immunological entity and the conserved regions of the second immunological entity in the three-dimensional structure models;
(4) determining similarity between non-conserved regions of the first immunological entity and non-conserved regions of the second immunological entity in the three-dimensional structure models after the superimposition; and
(5) judging whether an epitope binding to the first immunological entity and an epitope binding to the second immunological entity are identical or different based on the similarity.

2. The method of claim 1, wherein the immunological entity is an antibody, an antigen binding fragment of an antibody, a B cell receptor, a fragment of a B cell receptor, a T cell receptor, a fragment of a T cell receptor, a chimeric antigen receptor (CAR), or a cell comprising any one or more of them.

3. The method of claim 1, wherein identical residue are defined in determining the similarity.

4. The method of claim 1, wherein the similarity is determined based on at least one of a difference in lengths, sequence similarity, and three-dimensional structural similarity.

5. The method of claim 1, wherein the similarity comprises at least three-dimensional structural similarity.

6. A program for making a computer execute the method of claim 1.

7. A recording medium storing a program for making a computer execute the method of claim 1.

8. A system comprising a program for making a computer execute the method of claim 1.

9. The method of claim 1, comprising the step comprising associating the epitope with biological information.

10. A method for generating a cluster of epitopes, comprising the step of classifying immunological entities binding to an identical epitope to an identical cluster using the classification method of claim 1 or 9.

11. A method for identifying a disease, disorder, or biological condition, comprising the step of associating a carrier of the immunological entity with a known disease, disorder, or biological condition based on a cluster generated by the method of claim 10.

12. A composition for identifying the biological information, comprising an immunological entity to an epitope identified based on claim 11.

13. A composition for diagnosing the disease, disorder, or biological condition of claim 11, comprising an immunological entity to an epitope identified based on claim 1.

14. A composition for treating or preventing the disease, disorder, or biological condition of claim 11, comprising an immunological entity to an epitope identified based on claim 1.

15. The composition of claim 14, wherein the composition comprises a vaccine.
